SEQUENCE LISTING (52 sequences)

SEQ ID NO: 1 - protein, 883 aa, Saccharomyces cerevisiae
MQKIMHISVLLSPVLWGLIFGVSSNSIQIGGLFPRGADQEYSAFRVGMVQFSTSEFRLTPHIDNLEVANSFAVTNAFCSQFSRGVYAIFGFYDKKSVNTITSFCGTLHVSFITPSFPTDGTHPFVIQMRPDLKGALLSLIEYYQWDKFAYLYDSDRGLSTLQAVLDSAAEKKWQVTAINVGNINNDKKDEMYRSLFQDLELKKERRVILDCERDKVNDIVDQVITIGKHVKGYHYIIANLGFTDGDLLKIQFGGANVSGFQIVDYDDSLVSKFIERWSTLEEKEYPGAHTTTIKYTSALTYDAVQVMTEAFRNLRKQRIEISRRGNAGDCLANPAVPWGQGVEIERALKQVQVEGLSGNIKFDQNGKRINYTINIMELKTNGPRKIGYWSEVDKMVVTLTELPSGNDTSGLENKTVVVTTILESPYVMMKKNHEMLEGNERYEGYCVDLAAEIAKHCGFKYKLTIVGDGKYGARDADTKIWNGMVGELVYGKADIAIAPLTITLVREEVIDFSKPFMSLGISIMIKKPQKSKPGVFSFLDPLAYEIWMCIVFAYIGVSVVLFLVSRFSPYEWHTEEFEDGRETQSSESTNEFGIFNSLWFSLGAFMQQGCDISPRSLSGRIVGGVWWFFTLIIISSYTANLAAFLTVERMVSPIESAEDLSKQTEIAYGTLDSGSTKEFFRRSKIAVFDKMWTYMRSAEPSVFVRTTAEGVARVRKSKGKYAYLLESTMNEYIEQRKPCDTMKVGGNLDSKGYGIATPKGSSLRNAVNLAVLKLNEQGLLDKLKNKWWYDKGECGSGGGDSKEKTSALSLSNVAGVFYILVGGLGLAMLVALIEFCYKSRAEAKRMKVAKNAQNINPSSSQNSQNFATYKEGYNVYGIESVKI

SEQ ID NO: 2 - protein, 951 aa, synthetic construct
MTDSKYFTTNKKGEIFELKAELNNEKKEKRKEAVKKVIAAMTVGKDVSSLFPDVVNCMQTDNLELKKLVYLYLMNYAKSQPDMAIMAVNSFVKDCEDPNPLIRALAVRTMGCIRVDKITEYLCEPLRKCLKDEDPYVRKTAAVCVAKLHDINAQMVEDQGFLDSLRDLIADSNPMVVANAVAALSEISESHPNSNLLDLNPQNINKLLTALNECTEWGQIFILDCLSNYNPKDDREAQSICERVTPRLSHANSAVVLSAVKVLMKFPELLPKDSDYYNMLLKKLAPPLVTLLSGEPEVQYVALRNINLIVQKRPEILKQEIKVFFVKYNDPIYVKLEKLDIMIRLASQANIAQVLAELKEYATEVDVDFVRKAVRAIGRCAIKVEQSAERCVSTLLDLIQTKVNYVVQEAIVVIRDIFRKYPNKYESIIATLCENLDSLDEPDARAAMIWIVGEYAERIDNADELLESFLEGFHDESTQVQLTLLTAIVKLFLKKPSETQELVQQVLSLATQDSDNPDLRDRGYIYWRLLSTDPVTAKEVVLSEKPLISEETDLIEPTLLDELICHIGSLASVYHKPPNAFVEGSHGIHRKHLPIHHGSTDAGDSPVGTTTATNLEQPQVIPSQGDLLGDLLNLDLGPPVNVPQVSSMQMGAVDLLGGGLDSLLGSDLGGGIGGSPAVGQSFIPSSVPATFAPSPTPAVVSSGLNDLFELSTGIGMAPGGYVAPKAVWLPAVKAKGLEISGTFTHRQGHIYMEMNFTNKALQHMTDFAIQFNKNSFGVIPSTPLAIHTPLMPNQSIDVSLPLNTLGPVMKMEPLNNLQVAVKNNIDVFYFSCLIPLNVLFVEDGKMERQVFLATWKDIPNENELQFQIKECHLNADTVSSKLQNNNVYTIAKRNVEGQDMLYQSLKLTNGIWILAELRIQPGNPNYTLSLKCRAPEVSQYIYQVYDSILKN

SEQ ID NO: 3 - protein, 10 aa, Xenopus laevis
KRPAATKKXX

SEQ ID NO: 4 - protein, 11 aa, Xenopus laevis
XXGQAKKKKLD

SEQ ID NO: 5 - protein, 18 aa, Xenopus laevis
KRPAATKKAGQAKKKKLD

SEQ ID NO: 6 - protein, 17 aa, Homo sapiens
KRSAEGSNPPKPLKKLR

SEQ ID NO: 7 - protein, 7 aa, Homo sapiens
KRSAEGS

SEQ ID NO: 8 - protein, 10 aa, Homo sapiens
NPPKPLKKLR

SEQ ID NO: 9 - protein, 18 aa, Homo sapiens
KRALPNNTSSSPQPKKKP

SEQ ID NO: 10 - protein, 11 aa, Homo sapiens
KRALPNNTSSS

SEQ ID NO: 11 - protein, 7 aa, Homo sapiens
PQPKKKP

SEQ ID NO: 12 - protein, 184 aa, Saccharomyces cerevisiae
CFAKGTNVLMADGSIECIENIEVGNKVMGKDGRPREVIKLPRGRETMYSVVQKSQHRAHKSDSSREVPELLKFTCNATHELVVRTPRSVRRLSRTIKGVEYFEVITFEMGQKKAPDGRIVELVKEVSKSYPISEGPERANELVESYRKASNKAYFEWTIEARDLSLLGSHVRKATYQTYAPILY

SEQ ID NO: 13 - protein, 69 aa, Saccharomyces cerevisiae
MVLLNVLSKCAGSKKFRPAPAAAFARECRGFYFELQELKEDDYYGITLSDDSDHQFLLANQVVVHNCFN

SEQ ID NO: 14 - protein, 102 aa, Nostoc punctiforme
CLSYETEILTVEYGLLPIGKIVEKRIECTVYSVDNNGNIYTQPVAQWHDRGEQEVFEYCLEDGSLIRATKDHKFMTVDGQMLPIDEIFERELDLMRVDNLPN

SEQ ID NO: 15 - protein, 35 aa, Nostoc punctiforme
IKIATRKYLGKQNVYDIGVERDHNFALKNGFIASN

SEQ ID NO: 16 - protein, 88 aa, Saccharomyces cerevisiae
CLDLKTQVQTPQGMKEISNIQVGDLVLSNTGYNEVLNVFPKSKKKSYKITLEDGKEIICSEEHLFPTQTGEMNISGGLKEGMCLYVKE

SEQ ID NO: 17 - protein, 35 aa, Saccharomyces cerevisiae
LKKILKIEELDERELIDIEVSGNHLFYANDILTHN

SEQ ID NO: 18 - skipped

SEQ ID NO: 19 - protein, 1405 aa, synthetic construct
MKRPAATKKHMCFAKGTNVLMADGSIECIENIEVGNKVMGKDGRPREVIKLPRGRETMYSVVQKSQHRAHKSDSSREVPELLKFTCNATHELVVRTPRSVRRLSRTIKGVEYFEVITFEMGQKKAPDGRIVELVKEVSKSYPISEGPERANELVESYRKASNKAYFEWTIEARDLSLLGSHVRKATYQTYAPILYSRTDSKYFTTNKKGEIFELKAELNNEKKEKRKEAVKKVIAAMTVGKDVSSLFPDVVNCMQTDNLELKKLVYLYLMNYAKSQPDMAIMAVNSFVKDCEDPNPLIRALAVRTMGCIRVDKITEYLCEPLRKCLKDEDPYVRKTAAVCVAKLHDINAQMVEDQGFLDSLRDLIADSNPMVVANAVAALSEISESHPNSNLLDLNPQNINKLLTALNECTEWGQIFILDCLSNYNPKDDREAQSICERVTPRLSHANSAVVLSAVKVLMKFPELLPKDSDYYNMLLKKLAPPLVTLLSGEPEVQYVALRNINLIVQKRPEILKQEIKVFFVKYNDPIYVKLEKLDIMIRLASQANIAQVLAELKEYATEVDVDFVRKAVRAIGRCAIKVEQSAERCVSTLLDLIQTKVNYVVQEAIVVIRDIFRKYPNKYESIIATLCENLDSLDEPDARAAMIWIVGEYAERIDNADELLESFLEGFHDESTQVQLTLLTAIVKLFLKKPSETQELVQQVLSLATQDSDNPDLRDRGYIYWRLLSTDPVTAKEVVLSEKPLISEETDLIEPTLLDELICHIGSLASVYHKPPNAFVEGSHGIHRKHLPIHHGSTDAGDSPVGTTTATNLEQPQVIPSQGDLLGDLLNLDLGPPVNVPQVSSMQMGAVDLLGGGLDSLLGSDLGGGIGGSPAVGQSFIPSSVPATFAPSPTPAVVSSGLNDLFELSTGIGMAPGGYVAPKAVWLPAVKAKGLEISGTFTHRQGHIYMEMNFTNKALQHMTDFAIQFNKNSFGVIPSTPLAIHTPLMPNQSIDVSLPLNTLGPVMKMEPLNNLQVAVKNNIDVFYFSCLIPLNVLFVEDGKMERQVFLATWKDIPNENELQFQIKECHLNADTVSSKLQNNNVYTIAKRNVEGQDMLYQSLKLTNGIWILAELRIQPGNPNYTLSLKCRAPEVSQYIYQVYDSILKNGSGATNFSLLKQAGDVEENPGPMVSKGEEDNMAIIKEFMRFKVHMEGSVNGHEFEIEGEGEGRPYEGTQTAKLKVTKGGPLPFAWDILSPQFMYGSKAYVKHPADIPDYLKLSFPEGFKWERVMNFEDGGVVTVTQDSSLQDGEFIYKVKLRGTNFPSDGPVMQKKTMGWEASSERMYPEDGALKGEIKQRLKLKDGGHYDAEVKTTYKAKKPVQLPGAYNVNIKLDITSHNEDYTIVEQYERAEGRHSTGGMDELYK

SEQ ID NO: 20 - protein, 1204 aa, synthetic construct
MQKIMHISVLLSPVLWGLIFGVSSNSIQIGGLFPRGADQEYSAFRVGMVQFSTSEFRLTPHIDNLEVANSFAVTNAFCSQFSRGVYAIFGFYDKKSVNTITSFCGTLHVSFITPSFPTDGTHPFVIQMRPDLKGALLSLIEYYQWDKFAYLYDSDRGLSTLQAVLDSAAEKKWQVTAINVGNINNDKKDEMYRSLFQDLELKKERRVILDCERDKVNDIVDQVITIGKHVKGYHYIIANLGFTDGDLLKIQFGGANVSGFQIVDYDDSLVSKFIERWSTLEEKEYPGAHTTTIKYTSALTYDAVQVMTEAFRNLRKQRIEISRRGNAGDCLANPAVPWGQGVEIERALKQVQVEGLSGNIKFDQNGKRINYTINIMELKTNGPRKIGYWSEVDKMVVTLTELPSGNDTSGLENKTVVVTTILESPYVMMKKNHEMLEGNERYEGYCVDLAAEIAKHCGFKYKLTIVGDGKYGARDADTKIWNGMVGELVYGKADIAIAPLTITLVREEVIDFSKPFMSLGISIMIKKPQKSKPGVFSFLDPLAYEIWMCIVFAYIGVSVVLFLVSRFSPYEWHTEEFEDGRETQSSESTNEFGIFNSLWFSLGAFMQQGCDISPRSLSGRIVGGVWWFFTLIIISSYTANLAAFLTVERMVSPIESAEDLSKQTEIAYGTLDSGSTKEFFRRSKIAVFDKMWTYMRSAEPSVFVRTTAEGVARVRKSKGKYAYLLESTMNEYIEQRKPCDTMKVGGNLDSKGYGIATPKGSSLRNAVNLAVLKLNEQGLLDKLKNKWWYDKGECGSGGGDSKEKTSALSLSNVAGVFYILVGGLGLAMLVALIEFCYKSRAEAKRMKVAKNAQNINPSSSQNSQNFATYKEGYNVYGIESVKITRMVLLNVLSKCAGSKKFRPAPAAAFARECRGFYFELQELKEDDYYGITLSDDSDHQFLLANQVVVHNCFNVDGQAKKKKLDMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 21 - DNA, 12242 bp, synthetic construct
  source 1..12242: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccaggcaagtttgtacaaaaaagcaggctgccaccatgaaacgtccggcggccaccaagaaacatatgtgctttgccaagggtaccaatgttttaatggcggatgggtctattgaatgtattgaaaacattgaggttggtaataaggtcatgggtaaagatggcagacctcgtgaggtaattaaattgcccagaggaagagaaactatgtacagcgtcgtgcagaaaagtcagcacagagcccacaaaagtgactcaagtcgtgaagtgccagaattactcaagtttacgtgtaatgcgacccatgagttggttgttagaacacctcgtagtgtccgccgtttgtctcgtaccattaagggtgtcgaatattttgaagttattacttttgagatgggccaaaagaaagcccccgacggtagaattgttgagcttgtcaaggaagtttcaaagagctacccaatatctgaggggcctgagagagccaacgaattagtagaatcctatagaaaggcttcaaataaagcttattttgagtggactattgaggccagagatctttctctgttgggttcccatgttcgtaaagctacctaccagacttacgctccaattctttattctagaactgactccaagtatttcacaaccaataaaaaaggagaaatatttgaactaaaagctgaactcaacaatgaaaagaaagaaaagagaaaggaggctgtgaagaaagtgattgctgctatgaccgtggggaaggatgttagttctctctttccagacgtagtgaactgtatgcagactgacaatctggaactaaagaagcttgtgtatctctacttgatgaactacgccaagagtcagccagacatggccatcatggctgtaaacagctttgtgaaggactgtgaagatcctaatcctttgattcgagccttggcagtcagaaccatggggtgcatccgggtagacaaaattacagaatatctctgtgagccgctccgcaagtgcttgaaggatgaggatccctatgttcggaaaacagcagcagtctgcgtggcaaaactccatgatatcaatgcccaaatggtggaagatcagggatttctggattctctacgggatctcatagcagattcaaatccaatggtggtggctaatgccgtagcggcattatctgaaatcagtgagtctcacccaaacagcaacttacttgatctgaacccacagaacattaataagctgctgacagccctgaatgaatgcactgaatggggccagattttcatcctggactgcctgtctaattacaaccctaaagatgatcgggaggctcagagcatctgtgagcgggtaactccccggctatcccatgccaactcagcagtggtgctttcagcggtaaaagtcctaatgaagtttccagaattgttacctaaggattctgactactacaatatgctgctgaagaagttagcccctccacttgtcactttgctgtctggggagccagaagtgcagtatgtcgccctgaggaacatcaacttaattgtccagaaaaggcctgaaatcttgaagcaggaaatcaaagtcttctttgtgaagtacaatgatcccatctatgttaaactagagaagttggacatcatgattcgtttggcatctcaagccaacattgctcaggttctggcagaactgaaagaatatgctacagaggtggatgttgactttgttcgaaaagctgtgcgggccattggacggtgtgccatcaaggtggagcaatctgcagagcgctgtgtaagcacattgcttgatctaatccagaccaaagtgaattatgtggtccaagaagcaattgttgtcatcagggacatcttccgcaaataccccaacaagtatgaaagtatcatcgccactctgtgtgagaacttagactcgctggatgagccagatgctcgagcagctatgatttggattgtgggagaatatgctgaaagaattgacaatgcagatgagttactagaaagcttcctggagggttttcacgatgaaagcacccaggtgcagctcactctgcttactgccatagtgaagctgtttctcaagaaaccatcagaaacacaggagctagtccagcaggtcttgagtttggcaacacaggattctgataatcctgaccttcgagaccggggctatatttattggcgccttctctcaactgaccctgttacagctaaagaagtagtcttgtctgagaagccactgatctctgaggagacggaccttattgagccaactctgctggatgagctaatctgccacattggttctttggcctctgtgtatcataagcctcccaatgcttttgtggaaggaagtcatggaattcatcgtaaacacttgccaattcatcatgggagcactgatgcaggtgacagccctgttggcactaccactgcaacgaacctggaacagcctcaggttatcccctctcaaggtgatcttctaggggatcttttaaaccttgacctcggtcccccagtcaatgtgccacaggtgtcctccatgcagatgggagcagtggatctcctaggaggaggactagatagtctgcttggcagtgaccttggcgggggcattggaggaagtccggcagtgggacaatccttcatcccatcatcggtccctgcaacctttgctccttcacctacacctgctgtggtcagcagtggactgaatgacctgtttgaactctccacagggataggcatggcacctggtggatatgtggctcctaaggctgtctggctacctgcagtaaaggctaaaggcttggagatttccggaacatttactcaccgccaagggcacatctatatggaaatgaacttcaccaataaagctctgcagcacatgacagattttgcaatccagtttaacaaaaatagctttggtgtcatccccagcactcctctggccatccatacaccactgatgccaaaccagagcattgatgtctccctgcctctcaataccttgggcccagtcatgaagatggaacctctgaataacctccaggtggctgtgaaaaacaatatcgatgtcttctacttcagctgcctcatcccactcaatgtgctttttgtagaagatggcaaaatggagcgccaggtcttccttgcaacatggaaggatattcccaatgaaaatgaacttcagtttcagattaaggaatgtcatttaaatgctgacactgtttccagcaagttgcaaaacaacaatgtttatactattgccaagaggaatgtggaagggcaggacatgctgtaccaatccctgaagctcactaatggcatttggattttggccgaactacgtatccagccaggaaaccccaattacacgctgtcactgaagtgtagagctcctgaagtctctcaatacatctatcaggtctacgacagcattttgaaaaacggaagcggagccacgaacttctctctgttaaagcaagcaggagatgttgaagaaaaccccgggcctatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtaaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctcacgtgcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctgggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 22 - DNA, 12110 bp, synthetic construct
  source 1..12110: mol_type = other DNA
aatgtagtcttatgcaatactcttgtagtcttgcaacatggtaacgatgagttagcaacatgccttacaaggagagaaaaagcaccgtgcatgccgattggtggaagtaaggtggtacgatcgtgccttattaggaaggcaacagacgggtctgacatggattggacgaaccactgaattgccgcattgcagagatattgtatttaagtgcctagctcgatacataaacgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacttgaaagcgaaagggaaaccagaggagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcgctagcttttaaaagaaaaggggggattggggggtacagtgcaggggaaagaatagtagacataatagcaacagacatacaaactaaagaattacaaaaacaaattacaaaaattcaaaattttactagtgattatcggatcaactttgtatagaaaagttgggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggtctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacaagtttgtacaaaaaagcaggctgccaccatgcaaaagattatgcatatttctgtcctcctttctcctgttttatggggactgatttttggtgtctcttctaacagcatacagataggggggctatttcctaggggcgccgatcaagaatacagtgcatttcgagtagggatggttcagttttccacttcggagttcagactgacaccccacatcgacaatttggaggtggcaaacagcttcgcagtcactaatgctttctgctcccagttttcgagaggagtctatgctatttttggattttatgacaagaagtctgtaaataccatcacatcattttgcggaacactccacgtctccttcatcactcccagcttcccaacagatggcacacatccatttgtcattcagatgagacccgacctcaaaggagctctccttagcttgattgaatactatcaatgggacaagtttgcatacctctatgacagtgacagaggcttatcaacactgcaagctgtgctggattctgctgctgaaaagaaatggcaagtgactgctatcaatgtgggaaacattaacaatgacaagaaagatgagatgtaccgatcactttttcaagatctggagttaaaaaaggaacggcgtgtaattctggactgtgaaagggataaagtaaacgacattgtagaccaggttattaccattggaaaacatgttaaagggtaccactacatcattgcaaatctgggatttactgatggagacctattaaaaatccagtttggaggtgcaaatgtctctggatttcagatagtggactatgatgattcgttggtatctaaatttatagaaagatggtcaacactggaagaaaaagaataccctggagctcacacaacaacaattaagtatacttctgctctgacctatgatgccgttcaagtgatgactgaagccttccgcaacctaaggaagcaaagaattgaaatctcccgaagggggaatgcaggagactgtctggcaaacccagcagtgccctggggacaaggtgtagaaatagaaagggccctcaaacaggttcaggttgaaggtctctcaggaaatataaagtttgaccagaatggaaaaagaataaactatacaattaacatcatggagctcaaaactaatgggccccggaagattggctactggagtgaagtggacaaaatggttgttacccttactgagctcccttctggaaatgacacctctgggcttgagaataagactgttgttgtcaccacaattttggaatctccgtatgttatgatgaagaaaaatcatgaaatgcttgaaggcaatgagcgctatgagggctactgtgttgacctggctgcagaaatcgccaaacattgtgggttcaagtacaagttgacaattgttggtgatggcaagtatggggccagggatgcagacacgaaaatttggaatgggatggttggagaacttgtatatgggaaagctgatattgcaattgctccattaactattacccttgtgagagaagaggtgattgacttctcaaagcccttcatgagcctcgggatatctatcatgatcaagaagcctcagaagtccaaaccaggagtgttttcctttcttgatcctttagcctatgagatctggatgtgcattgtttttgcctacattggggtcagtgtagttttattcctggtcagcagatttagcccctacgagtggcacactgaggagtttgaagatggaagagaaacacaaagtagtgaatcaactaatgaatttgggatttttaatagtctctggttttccttgggtgcctttatgcagcaaggatgcgatatttcgccaagatccctctctgggcgcattgttggaggtgtgtggtggttctttaccctgatcataatctcctcctacacggctaacttagctgccttcctgactgtagagaggatggtgtctcccatcgaaagtgctgaggatctttctaagcaaacagaaattgcttatggaacattagactctggctccactaaagagtttttcaggagatctaaaattgcagtgtttgataaaatgtggacctacatgcggagtgcggagccctctgtgtttgtgaggactacggccgaaggggtggctagagtgcggaagtccaaagggaaatatgcctacttgttggagtccacgatgaacgagtacattgagcaaaggaagccttgcgacaccatgaaagttggtggaaacctggattccaaaggctatggcatcgcaacacctaaaggatcctcattaagaaatgcggttaacctcgcagtactaaaactgaatgaacaaggcctgttggacaaattgaaaaacaaatggtggtacgacaaaggagagtgcggcagcgggggaggtgattccaaggaaaagaccagtgccctcagtctgagcaacgttgctggagtattctacatccttgtcgggggccttggtttggcaatgctggtggctttgattgagttctgttacaagtcaagggccgaggcgaaacgaatgaaggtggcaaagaatgcacagaatattaacccatcttcctcgcagaattcacagaattttgcaacttataaggaaggttacaacgtatatggcatcgaaagtgttaaaattacgcgtatggttttgcttaacgttctttcgaagtgtgccggctctaaaaaattcaggcctgctcccgccgctgcttttgcacgtgagtgccgcggattttatttcgagttacaagaattgaaggaagacgattattatgggattactttatctgatgattctgatcatcagtttttgcttgcaaaccaggttgtcgtccataactgtttcaacgtcgacggccaggcgaaaaagaagaaactggatatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaacccagctttcttgtacaaagtggtgataatcgaattccgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgggaattcccgcggttcgaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctcacgtggccaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgaggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctagggacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagagagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagctt

SEQ ID NO: 23 - protein, 22 aa, synthetic construct
GSGATNFSLLKQAGDVEENPGP

SEQ ID NO: 24 - RNA, 15 nt, Human immunodeficiency virus 1
  source 1..15: mol_type = genomic RNA
aaaagaaaagggggg

SEQ ID NO: 25 - DNA, 28 bp, synthetic construct
  source 1..28: mol_type = other DNA
gagtgttcaccggtactagtatggtttt

SEQ ID NO: 26 - DNA, 23 bp, synthetic construct
  source 1..23: mol_type = other DNA
tcaccatgggcccatccagtttc

SEQ ID NO: 27 - DNA, 21 bp, synthetic construct
  source 1..21: mol_type = other DNA
gggcccatggtgagcaagggc

SEQ ID NO: 28 - DNA, 25 bp, synthetic construct
  source 1..25: mol_type = other DNA
tctagattacttgtacagctcgtcc

SEQ ID NO: 29 - DNA, 27 bp, synthetic construct
  source 1..27: mol_type = other DNA
gtggatgctaccggtaccatggaaatg

SEQ ID NO: 30 - DNA, 28 bp, synthetic construct
  source 1..28: mol_type = other DNA
caaggtcatactagtaattttaacactt

SEQ ID NO: 31 - DNA, 35 bp, synthetic construct
  source 1..35: mol_type = other DNA
cacattaaagatgtcgactcatgactgactccaag

SEQ ID NO: 32 - DNA, 34 bp, synthetic construct
  source 1..34: mol_type = other DNA
gtactggaccacggccgttagtttttcaaaatgc

SEQ ID NO: 33 - DNA, 25 bp, synthetic construct
  source 1..25: mol_type = other DNA
gcggcacgcgtatgaaacgtccggc

SEQ ID NO: 34 - DNA, 28 bp, synthetic construct
  source 1..28: mol_type = other DNA
gagtgcggccgttagtttttcaaaatgc

SEQ ID NO: 35 - DNA, 77 bp, synthetic construct
  source 1..77: mol_type = other DNA
tcgacatgaaacgtccggcggccaccaagaaagctagctgtttcaacgctagcggccaggcgaaaaagaagaaactg

SEQ ID NO: 36 - DNA, 77 bp, synthetic construct
  source 1..77: mol_type = other DNA
cagtttcttctttttcgcctggccgctagcgttgaaacagctagctttcttggtggccgccggacgtttcatgtcga

SEQ ID NO: 37 - DNA, 24 bp, synthetic construct
  source 1..24: mol_type = other DNA
ggctcgaagcttatgggcaagcag

SEQ ID NO: 38 - DNA, 25 bp, synthetic construct
  source 1..25: mol_type = other DNA
gaacctgggatatctcagaactggg

SEQ ID NO: 39 - DNA, 20 bp, synthetic construct
  source 1..20: mol_type = other DNA
ccgcatcttcttttgcgtcg

SEQ ID NO: 40 - DNA, 21 bp, synthetic construct
  source 1..21: mol_type = other DNA
gcccaatacgaccaaatccgt

SEQ ID NO: 41 - DNA, 20 bp, synthetic construct
  source 1..20: mol_type = other DNA
cgagtggaaacttttgtcgg

SEQ ID NO: 42 - DNA, 20 bp, synthetic construct
  source 1..20: mol_type = other DNA
tcttcatgagcgtcttggtt

SEQ ID NO: 43 - DNA, 21 bp, synthetic construct
  source 1..21: mol_type = other DNA
ggaccctgtgagtagccagta

SEQ ID NO: 44 - DNA, 19 bp, synthetic construct
  source 1..19: mol_type = other DNA
cagctccgacagatccagt

SEQ ID NO: 45 - DNA, 21 bp, synthetic construct
  source 1..21: mol_type = other DNA
gtctccttcgttcagacgctt

SEQ ID NO: 46 - DNA, 21 bp, synthetic construct
  source 1..21: mol_type = other DNA
aaagtccgaggattgagttgc

SEQ ID NO: 47 - DNA, 20 bp, synthetic construct
  source 1..20: mol_type = other DNA
agatggtttcgtccgtgatg

SEQ ID NO: 48 - DNA, 19 bp, synthetic construct
  source 1..19: mol_type = other DNA
cttgccgtcgttgtttgtg

SEQ ID NO: 49 - protein, 340 aa, Saccharomyces cerevisiae
YEIWMCIVFAYIGVSVVLFLVSRFSPYEWHTEEFEDGRETQSSESTNEFGIFNSLWFSLGAFMQQGCDISPRSLSGRIVGGVWWFFTLIIISSYTANLAAFLTVERMVSPIESAEDLSKQTEIAYGTLDSGSTKEFFRRSKIAVFDKMWTYMRSAEPSVFVRTTAEGVARVRKSKGKYAYLLESTMNEYIEQRKPCDTMKVGGNLDSKGYGIATPKGSSLRNAVNLAVLKLNEQGLLDKLKNKWWYDKGECGSGGGDSKEKTSALSLSNVAGVFYILVGGLGLAMLVALIEFCYKSRAEAKRMKVAKNAQNINPSSSQNSQNFATYKEGYNVYGIESVKI

SEQ ID NO: 50 - protein, 292 aa, Saccharomyces cerevisiae
FGIFNSLWFSLGAFMQQGCDISPRSLSGRIVGGVWWFFTLIIISSYTANLAAFLTVERMVSPIESAEDLSKQTEIAYGTLDSGSTKEFFRRSKIAVFDKMWTYMRSAEPSVFVRTTAEGVARVRKSKGKYAYLLESTMNEYIEQRKPCDTMKVGGNLDSKGYGIATPKGSSLRNAVNLAVLKLNEQGLLDKLKNKWWYDKGECGSGGGDSKEKTSALSLSNVAGVFYILVGGLGLAMLVALIEFCYKSRAEAKRMKVAKNAQNINPSSSQNSQNFATYKEGYNVYGIESVKI

SEQ ID NO: 51 - protein, 71 aa, Saccharomyces cerevisiae
VAGVFYILVGGLGLAMLVALIEFCYKSRAEAKRMKVAKNAQNINPSSSQNSQNFATYKEGYNVYGIESVKI

SEQ ID NO: 52 - protein, 239 aa, Aequorea victoria
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

(19) United States
(12) Patent Application Publication
(10) Pub. No.: US 2026/0036580 A1
(43) Pub. Date: Feb. 5, 2026

(54) USE OF STEM CELL-BASED BIOSENSORS FOR REAL-TIME, NON-INVASIVE MONITORING OF NEURONAL DIFFERENTIATION

(71) Applicants: Rutgers, The State University of New Jersey, New Brunswick, NJ (US); Dongguk University, South Korea, Goyang-Si (KR)

(72) Inventors: Euiyeon Lee, Somerset, NJ (US); Ki-Bum Lee, Monmouth Junction, NJ (US); Youngeun Kwon, Kyounggi-do (KR)

(73) Assignees: Rutgers, The State University of New Jersey, New Brunswick, NJ (US); Dongguk University, South Korea, Goyang-Si (KR)

(21) Appl. No.: 19/249,581

(22) Filed: Jun. 25, 2025

Related U.S. Application Data
(60) Provisional application No. 63/663,883, filed on Jun. 25, 2024.

(51) Int. Cl. G01N 33/566; C07K 5/08; C07K 14/705; C07K 19/00; C12N 15/86

(52) U.S. Cl. CPC G01N 33/566; C07K 5/0802; C07K 14/705; C07K 19/00; C12N 15/86; C07K 2319/09; C07K 2319/60; C07K 2319/92; C12N 2740/15043

(57) ABSTRACT

Compositions and methods for real-time non-invasive monitoring of neuronal differentiation. Biosensor proteins comprising N-fusion and C-fusion proteins the express Adaptor Protein Complex 2 (AP2) and glutamate ionotropic receptor AMPA type subunit 2 (GRIA2), respectively, as well as split inteins and a reporter molecule enable the detection of hippocalcin via the translocation of a fluorescent protein into the nucleus of a cell.

### CROSS-REFERENCE TO RELATED APPLICATIONS

This application claims priority under 32 U.S.C. § 119(e) to U.S. Provisional Patent Application No. 63/663,883 filed Jun. 25, 2024. The entire disclosure of the application noted above is incorporated herein by reference

### STATEMENT REGARDING FEDERALLY SPONSORED RESEARCH

This invention was made with government support under grant number 1R01DC016612 awarded by the National Institutes of Health (NIH) and under grant number CBET-1803517 awarded by the National Science Foundation (NSF). The government has certain rights in the invention.

### FIELD OF THE INVENTION

This invention relates to compositions and methods of use thereof to monitor neuronal differentiation.

### BACKGROUND OF THE INVENTION

Monitoring neuronal differentiation in stem cells is highly important to advance regenerative stem cell therapies and improve our understanding of the complex processes involved in neural development. This knowledge is essential for evaluating the progression of neurodegenerative diseases. The effectiveness of stem cell therapies for neurological disorders, which are characterized by neuronal loss or damage (such as Alzheimer's disease, Parkinson's disease, stroke, and traumatic brain injury), relies on the precise regulation of neuronal differentiation in neural stem cells (NSCs) in vitro and/or in vivo. Despite the high clinical potential of NSC therapy, its applications are limited by safety risks owing to heterogeneous cell populations. The pluripotent nature of stem cells allows differentiation into any cell type in the body, which is advantageous for various applications; however, this is associated with the risk of teratoma formation due to uncontrolled cell growth and differentiation. Additionally, differentiating cell types into undesirable lineages can generate tumors or unwanted tissues upon transplantation. The accurate identification and monitoring of neuronal differentiation progress and cell heterogeneity is crucial to ensure proper cell type specification and minimize the risk of tumor formation during stem cell differentiation protocols. However, this endeavor faces significant challenges due to the inherent limitations of traditional techniques, such as reverse transcription-polymerase chain reaction (RT-PCR), western blotting, and immunofluorescence (IF) staining. These methods, while valuable, often suffer from limitations such as low sensitivity, population averaging effects, or potential artifacts introduced during sample preparation and analysis, hindering our ability to fully grasp the intricate dynamics of neuronal differentiation and the potential presence of unwanted cell types. The aforementioned techniques are time-consuming, suitable for endpoint analysis, involve invasive or destructive measurements, and offer limited resolution to monitor differentiation at the single-cell level. Moreover, these techniques provide only snapshots of neuronal differentiation at specific time points, failing to capture the dynamic changes and gradual progression of the differentiation process. Therefore, there is an urgent need for more efficient, accurate, and convenient real-time monitoring method to evaluate neuronal differentiation non-invasively.

### SUMMARY OF THE INVENTION

The present application addresses this need and provides compositions and methods for real-time non-invasive monitoring of neuronal differentiation. In one aspect provided is a biosensor protein comprising a C-fusion protein and an N-fusion protein, wherein
i. the C-fusion protein comprises a nuclear localization signal (NLS), a C-intein, CFN tripeptide sequence (SEQ ID NO: 18), a glutamate ionotropic receptor AMPA type subunit 2 (GRIA2), and a reporter molecule; and
ii. the N-fusion protein comprises an NLS, an N-intein, and an Adaptor Protein Complex 2 (AP2).

According to one embodiment, the GRIA2 comprises the sequence of SEQ ID NO: 1. In one embodiment, the AP2 comprises the sequence of SEQ ID NO: 2.

In one embodiment, the C-intein comprises the sequence of any one of SEQ ID NOs: 13, 15, or 17. In one embodiment, the N-intein comprises the sequence of any one of SEQ ID NOs: 12, 14, or 16.

In one embodiment, the NLS sequence of the N-fusion protein comprises the sequence of any one of SEQ ID NOs: 3, 7, and 10. In one embodiment, the NLS sequence of the C-fusion protein comprises the sequence of any one of SEQ ID NOs: 4, 8, and 11.

In one embodiment, the reporter molecule is a fluorescent protein. In some embodiments, the fluorescent protein comprises yellow fluorescent protein (YFP), green fluorescent protein (GFP), mCherry, red fluorescent protein (RFP), or blue fluorescent protein (BFP). In one embodiment, the reporter molecule protein is inserted at the C-terminus of the NLS of the C-fusion protein. In one embodiment, the N-fusion protein further comprises a fluorescent protein inserted at the C-terminus of AP2.

In one embodiment, AP2 of the N-fusion protein binds to hippocalcin (HPCA). In one embodiment, the C-fusion and the N-fusion protein heterodimerize in response to hippocalcin binding AP2 of the C-fusion protein.

In one embodiment, provided is an isolated nucleic acid molecule encoding the biosensor protein as described herein. In one embodiment, the nucleic acid molecule encodes the biosensor protein as described herein. In one embodiment, provided is an expression vector or a host cell comprising the nucleic acid molecule described herein. In one embodiment, the host cell is a stem cell. In one embodiment, the stem cell, is a human induced pluripotent stem cell (hiPSC)-derived neural progenitor cell (NPC).

In another aspect, provided is a cell-based biosensor (CBB) that expresses:
i. a C-fusion protein, wherein the C-fusion protein comprises a nuclear localization signal (NLS), a C-intein, CFN tripeptide sequence (SEQ ID NO: 18), a glutamate ionotropic receptor AMPA type subunit 2 (GRIA2), and a reporter molecule; and
ii. an N-fusion protein, wherein the N-fusion protein comprises an NLS, an N-intein, and an Adaptor Protein Complex 2 (AP2).

In one embodiment, the GRIA2 comprises the sequence of SEQ ID NO: 1. In one embodiment, the AP2 comprises the sequence of SEQ ID NO: 2.

In one embodiment, the C-intein comprises the sequence of any one of SEQ ID NOs: 13, 15, or 17. In one embodiment, the N-intein comprises the sequence of any one of SEQ ID NOs: 12, 14, or 16.

In one embodiment, the NLS sequence of the N-fusion protein comprises the sequence of any one of SEQ ID NOs: 3, 7, and 10. In one embodiment, the NLS sequence of the C-fusion protein comprises the sequence of any one of SEQ ID NOs: 4, 8, and 11.

In one embodiment, the reporter molecule is a fluorescent protein. In some embodiments, the fluorescent protein comprises yellow fluorescent protein (YFP), green fluorescent protein (GFP), mCherry, red fluorescent protein (RFP), or blue fluorescent protein (BFP).

In one embodiment, the CBB detects hippocalcin. In one embodiment, the CBB is a stem cell. In one embodiment, the stem cell is a human induced pluripotent stem cell (hiPSC)-derived neural progenitor cell (NPC).

In another aspect, provided is a method for monitoring neuronal differentiation, the method comprising imaging a cell-based biosensor (CBB), wherein the CBB expresses:
i. a C-fusion protein, wherein the C-fusion protein comprises a nuclear localization signal (NLS), a C-intein, CFN tripeptide sequence (SEQ ID NO: 18), a glutamate ionotropic receptor AMPA type subunit 2 (GRIA2), and a reporter molecule; and
ii. an N-fusion protein, wherein the N-fusion protein comprises an NLS, an N-intein, and an Adaptor Protein Complex 2 (AP2); and wherein the CBB detects hippocalcin expression.

In one embodiment, the CBB emits a fluorescent signal when hippocalcin binds to AP2 of the N-fusion protein that results in intein-mediated conditional protein splicing (CPS). In one embodiment, the reporter molecule is a fluorescent protein. In some embodiments, the fluorescent protein comprises yellow fluorescent protein (YFP), green fluorescent protein (GFP), mCherry, red fluorescent protein (RFP), or blue fluorescent protein (BFP). In one embodiment, the imaging is live-cell imaging.

In one embodiment, the GRIA2 comprises the sequence of SEQ ID NO: 1. In one embodiment, the AP2 comprises the sequence of SEQ ID NO: 2.

In one embodiment, the C-intein comprises the sequence of any one of SEQ ID NOs: 13, 15, or 17. In one embodiment, the N-intein comprises the sequence of the amino acid sequence of any one of SEQ ID NOs: 12, 14, or 16.

In one embodiment, the NLS sequence of the N-fusion protein comprises the amino acid sequence of any one of SEQ ID NOs: 3, 7, and 10. In one embodiment, the NLS sequence of the C-fusion protein comprises the amino acid sequence of any one of SEQ ID NOs: 4, 8, and 11.

In one embodiment, the CBB is a stem cell. In one embodiment, the stem cell is a human induced pluripotent stem cell (hiPSC)-derived neural progenitor cell (NPC).

### BRIEF DESCRIPTION OF THE FIGURES

FIGS. 1A-1D are schematics of the working principle of cell-based biosensors (CBBs) for monitoring neuronal differentiation. FIG. 1A is a schematic that presents the need of CBBs. A non-invasive biosensing platform is required to monitor neuronal differentiation instead of a conventional approach, including RT-PCR, western blotting, and IF staining, for effective stem cell therapy for neurodegenerative diseases. FIG. 1B illustrates a non-invasive monitoring strategy for discrimination of neuronal differentiation from non-neuronal differentiation using CBBs. FIG. 1C shows the domain architecture of the DNA construct of the hippocalcin sensor proteins containing split N- or C-inteins. FIG. 1D is a schematic of monitoring neuronal differentiation via green fluorescence signal translocation.

FIG. 2 shows a sensing strategy to monitor neuronal differentiation via intracellular fluorescence signal translocation using intein-mediated conditional protein splicing (CPS) reaction.

FIGS. 3A-3G illustrate detection of hippocalcin using CBBs based on intein-mediated CPS reaction. FIG. 3A shows line-scan profiles of fluorescence (Fl) intensity in HeLa sensor cells expressing hippocalcin sensor proteins (N- and C-fusion). FIG. 3B is a graph of the green fluorescence intensity ratio (FIR) from the nucleus-to-cytoplasm (Nuc/Cyto) of the cells. FIG. 3C is a Western blot showing the formation of CPS product by the sensor cells in the presence of hippocalcin. FIG. 3D is a graph of the relative protein expression levels determined by western blotting in FIG. 3C. FIG. 3E is a dose-response curve showing the increase in the green FIR of Nuc/Cyto with increasing hippocalcin concentration; the LOD was 42 nm. FIG. 3F is a time-response curve of the green FIR (Nuc/Cyto) of the CBBs. FIG. 3G. shows line-scan profiles of Fl intensity in HEK293T cells expressing hippocalcin sensor protein.

FIGS. 4A-4E illustrate monitoring of neuronal differentiation using CBBs that express hippocalcin sensor proteins using hiPSC-NPCs. FIG. 4A is a timeline of experiments for monitoring of neuronal differentiation. Shaded squares indicate days observed via confocal microscopy (i.e., day 1, 2, 4, and 6). FIG. 4B shows line-scan profiles of Fl intensity in hiPSC-NPCs expressing sensor protein. Fl intensity profile across the white line is shown in the merged image. FIG. 4C shows green fluorescence intensity ratios from the nucleus to cytoplasm from IF images of the cells. FIG. 4D is western blot analysis that showed the formation of CPS product on days 2 and 6 during differentiation (one of two replicate experiments performed). FIG. 4E is a graph of the relative protein expression levels determined by western blotting (FIG. 4D).

FIGS. 5A-5E depict the generation of NSC-based CBBs stably expressing the hippocalcin sensor proteins, N- and C-fusion. FIG. 5A is a graphical summary of the procedure. FIG. 5B is a schematic representation of lentiviral vectors expressing N-fusion with mCherry and C-fusion used in this study, respectively. FIG. 5C are graphs of fluorescence-activated cell sorting analysis of the C-fusion-positive cell population. FIG. 5D shows fluorescent images of double-transduced NSCs expressing N- and C-fusion. FIG. 5E are graphs of NSCs that were segregated by gating on mCherry or EGFP. FSC-H, forward scatter height; FSC-A, forward scatter area (scale bar=15 μm).

FIGS. 6A-6G illustrate non-invasive monitoring of neural lineage differentiation using NSC-based CBBs that stably expressed the hippocalcin sensor protein. FIG. 6A is a timeline of the experiments for non-invasive monitoring of neuronal differentiation. hippocalcin sensor presenting NSCs are seeded for experiments on day −1. Shaded squares indicate days observed via confocal microscopy. FIG. 6B shows line-scan profiles of Fl intensity in sensor cells analyzed from IF images. FIG. 6C is a time-response curve demonstrated that the sensor started to respond from day 1 of differentiation, and the green FIR of Nuc/Cyto plateaued approximately at day 3 of differentiation. FIG. 6D is a western blot analysis that was performed to confirm CPS product formation on days 0, 1, 2, and 7 of differentiation (data are from one of two biological replicates performed). FIG. 6E is a graph of the relative protein expression levels determined by western blotting of FIG. 6D. FIG. 6F is a graph of the number of sensor cells with a green FIR of Nuc/Cyto >1. FIG. 6G is a graph of the green FIR of Nuc/Cyto that was assessed in sensor cells that stained positive for TuJ1, GFAP, and Olig2, respectively.

FIG. 7 shows a time-dependent curve analysis of mock sensor for detection of hippocalcin.

FIG. 8 depicts HPCA and neuronal fate gene (SOX2, NEUROD1, and TUBB3) expression in NSC-based CBBs by RT-PCR. Expression during differentiation of NSCs.

FIGS. 9A-9C show time-lapse imaging of green fluorescence translocation in NSCs stably expressing hippocalcin sensor. FIG. 9A shows the IF images of green fluorescence translocation in NSCs stably expressing hippocalcin sensor. FIG. 9B shows line scanning profiles of green fluorescence intensity across the selected cells marked by a white line were analyzed in FIG. 9A. FIG. 9C is a graph that shows that green FIR (Nuc/Cyto) in NSCs stably expressing hippocalcin sensor.

### DETAILED DESCRIPTION OF THE INVENTION

### 1. Overview

This disclosure relates to compositions and methods for real-time non-invasive monitoring of neuronal differentiation. In one aspect provided is a biosensor protein comprising a C-fusion protein and an N-fusion protein, wherein the biosensor protein detects hippocalcin. In one embodiment, the C-fusion protein comprises a nuclear localization signal (NLS), a C-intein, CFN tripeptide sequence (SEQ ID NO: 18), a glutamate ionotropic receptor AMPA type subunit 2 (GRIA2), and a reporter molecule; and the N-fusion protein comprises an NLS, an N-intein, and an Adaptor Protein Complex 2 (AP2). In one aspect, provided is CBB that express the biosensor protein described herein. In another aspect, provided is method for monitoring neuronal differentiation, the method comprising imaging a CBB described herein.

### 2. Hippocalcin

Hippocalcin (HPCA) belongs to the neuronal calcium (Ca²⁺) sensor family of proteins and is highly expressed in the hippocampus and cortex, which are important areas of the brain for learning and memory. Recent studies have reported that hippocalcin is essential for the neurogenesis of NSCs and promotes neuronal differentiation. The level of hippocalcin expression increases markedly under differentiation conditions, and overexpression of hippocalcin induces up-regulation of neurotrophin-3 (Nt3), neurotrophins 4 and 5 (Nt4/5), brain-derived neurotrophic factor (BDNF), Neuro-D (BETA2), and neurogenin-1 (NEUROG1). Nt3 supports the survival and differentiation of existing neurons and aids the differentiation and growth of new neurons. Nt4/5 promote the survival of both motor and sensory neurons. BDNF binds to its high-affinity receptor tyrosine kinase B (TrkB), which promotes differentiation, survival, and maturation of neurons. Neuro-D is a basic helix-loop-helix (bHLH) transcription factor and its expression is associated with neuronal differentiation. Neurogenin-1 is a transcriptional regulator and involved in the initiation of neuronal differentiation.

Adaptor Protein Complex 2 (AP2) directly binds to hippocalcin via the β2-adaptin subunit, in a calcium-sensitive manner. The N-terminus of AP2 is required for binding to hippocalcin. AP2 is heterotetrameric and consists of two large subunits (α and β2), a medium subunit (μ2), and a small subunit (σ2). AP2 is involved in clathrin-mediated endocytosis. Specifically, the μ2 subunit of AP2 (also termed AP50) is required for clathrin recruitment to sites of endocytosis. The AP2-hippocalcin complex combines with GRIA2 (glutamate ionotropic receptor AMPA type subunit 2) in hippocalcin-Ca²⁺ signaling. GRIA2 consists of an amino-terminal domain (ATD), a ligand-binding domain (LBD), a transmembrane domain (formed by hydrophobic membrane-spanning helices M1, M3 and M4 and the M2 helix and re-entrant loop) and a carboxy-terminal (CTD) intracellular region. Note that glutamate receptors function as ligand-activated cation channels that are formed from 4 subunits, GRIA1-4. The interaction of the AP2-hippocalcin complex with GRIA2 is induced by the myristoylation of hippocalcin, which is essential for the expression of nerve growth factors during neuronal differentiation of NSCs and is well-known to contribute to neuronal activities, including synaptic plasticity, learning, and memory.

### 3. Inteins

Inteins have a distinctive characteristic: they autonomously excise themselves and simultaneously ligate adjacent protein segments, called exteins, by forming an amide bond. This process is known as protein splicing (PS). Inteins are found in various organisms including fungi, bacteria, and lower plants. Additionally, inteins exist in different forms including mini-inteins, full-length inteins, and naturally split inteins. Split inteins are trans-splicing inteins that have two fragments transcribed and translated by two independent genes. Split inteins are described for example, in WO2013045632, the disclosure of which is incorporated by reference in its entirety.

In one embodiment, the N-fusion and C-fusion proteins described herein comprise split-inteins. One well studied split-intein is the vacuolar membrane ATPase subunit of Saccharomyces cerevisiae intein (Sce VMA intein) (Hirata et al. J. Biol. Chem. 1990. 265. 6726-6733).

In some embodiments, the N-fusion and C-fusion proteins described herein comprise the N-intein and C-intein pairs shown in Table 1.

TABLE 1
Exemplary N-intein and C-intein pairs.

| N-intein | C-intein |
|---|---|
| CFAKGTNVLMADGSIEC | MVLLNVLSKCAGSKKFR |
| IENIEVGNKVMGKDGRP | PAPAAAFARECRGFYFE |
| REVIKLPRGRETMYSVV | LQELKEDDYYGITLSDD |
| QKSQHRAHKSDSSREVP | SDHQFLLANQVVVHNCF |
| ELLKFTCNATHELVVRT | N |
| PRSVRRLSRTIKGVEYF | (SEQ ID NO: 13; |
| EVITFEMGQKKAPDGRI | Sce VMA C-intein) |
| VELVKEVSKSYPISEGP | |
| ERANELVESYRKASNKA | |
| YFEWTIEARDLSLLGSH | |
| VRKATYQTYAPILY | |
| (SEQ ID NO: 12; | |
| Sce VMA N-intein) | |
| CLSYETEILTVEYGLLP | IKIATRKYLGKQNVYDI |
| IGKIVEKRIECTVYSVD | GVERDHNFALKNGFIAS |
| NNGNIYTQPVAQWHDRG | N |
| EQEVFEYCLEDGSLIRA | (SEQ ID NO: 15; |
| TKDHKFMTVDGQMLPID | Npu DnaE C-intein) |
| EIFERELDLMRVDNLPN | |
| (SEQ ID NO: 14; | |
| Npu DnaE N-intein) | |
| CLDLKTQVQTPQGMKEI | LKKILKIEELDERELID |
| SNIQVGDLVLSNTGYNE | IEVSGNHLFYANDILTH |
| VLNVFPKSKKKSYKITL | N |
| EDGKEIICSEEHLFPTQ | (SEQ ID NO: 17; |
| TGEMNISGGLKEGMCLY | Gp41-1 C-intein) |
| VKE | |
| (SEQ ID NO: 16; | |
| Gp41-1 N-intein) | |

The Nostoc punctiforme (Npu) DnaE intein is a split-intein that works in trans. It is also one of the fastest known inteins (Cheriyan, M. et al., JBC. 2013. 288(9): 6202-6211). The GP41-1 intein is a split intein (H. M. Beyer et al., FEBS J. 2020. 287(9): 1886-1898).

In one embodiment, the N-fusion protein comprises the N-intein sequence of SEQ ID NO: 12. In one embodiment, the C-fusion protein comprises the C-intein sequence of SEQ ID NO: 13.

As used herein, “N-intein” is used interchangeably with “I_{N}.” Additionally, “C-intein is used interchangeably with “Ic.”

### 4. Biosensor Proteins

The biosensor proteins described herein are comprised of N-fusion and C-fusion proteins. In some embodiments, the N-fusion and C-fusion proteins comprise split-nuclear localization signal (NLS) peptides. The N-fusion and C-fusion proteins described herein comprise an N-intein and C-intein, respectively. Notably, the N-fusion and C-fusion proteins can heterodimerize upon detecting hippocalcin. Once hippocalcin is detected, the biosensor proteins emit a fluorescent signal.

The N-fusion and C-fusion proteins comprise proteins that detect hippocalcin. In one embodiment, the C-fusion protein comprises GRIA2 and the N-fusion protein comprises AP2. In this case, AP2 and GRIA2 are introduced separately into the C-terminus of N-intein (I_{N}) and N-terminus of C-intein (Ic) respectively, allowing for the heterodimerization of split-inteins by hippocalcin.

Notably, the biosensor proteins can detect hippocalcin expression earlier than class III β-tubulin (TuJ1) expression, which is a marker for neuronal differentiation.

### 4A. C-Fusion Proteins

In one embodiment, the C-fusion protein comprises an NLS C-fragment, a C-intein, CFN tripeptide sequence, a GRIA2, and a reporter molecule (from 5′ to 3′ orientation). The components of the C-fusion protein are operatively linked together.

Exemplary sequences of GRIA2 are shown below.

(GRIA2):
SEQ ID NO: 1
MQKIMHISVLLSPVLWGLIFGVSSNSIQIGGLFPRGADQEYSAFRVGMVQ
FSTSEFRLTPHIDNLEVANSFAVTNAFCSQFSRGVYAIFGFYDKKSVNTI
TSFCGTLHVSFITPSFPTDGTHPFVIQMRPDLKGALLSLIEYYQWDKFAY
LYDSDRGLSTLQAVLDSAAEKKWQVTAINVGNINNDKKDEMYRSLFQDLE
LKKERRVILDCERDKVNDIVDQVITIGKHVKGYHYIIANLGFTDGDLLKI
QFGGANVSGFQIVDYDDSLVSKFIERWSTLEEKEYPGAHTTTIKYTSALT
YDAVQVMTEAFRNLRKQRIEISRRGNAGDCLANPAVPWGQGVEIERALKQ
VQVEGLSGNIKFDQNGKRINYTINIMELKTNGPRKIGYWSEVDKMVVTLT
ELPSGNDTSGLENKTVVVTTILESPYVMMKKNHEMLEGNERYEGYCVDLA
AEIAKHCGFKYKLTIVGDGKYGARDADTKIWNGMVGELVYGKADIAIAPL
TITLVREEVIDFSKPFMSLGISIMIKKPQKSKPGVFSFLDPLAYEIWMCI
VFAYIGVSVVLFLVSRFSPYEWHTEEFEDGRETQSSESTNEFGIFNSLWF
SLGAFMQQGCDISPRSLSGRIVGGVWWFFTLIIISSYTANLAAFLTVERM
VSPIESAEDLSKQTEIAYGTLDSGSTKEFFRRSKIAVFDKMWTYMRSAEP
SVFVRTTAEGVARVRKSKGKYAYLLESTMNEYIEQRKPCDTMKVGGNLDS
KGYGIATPKGSSLRNAVNLAVLKLNEQGLLDKLKNKWWYDKGECGSGGGD
SKEKTSALSLSNVAGVFYILVGGLGLAMLVALIEFCYKSRAEAKRMKVAK
NAQNINPSSSQNSQNFATYKEGYNVYGIESVKI
(M2 to CTD of GRIA2)
SEQ ID NO: 49
YEIWMCIVFAYIGVSVVLFLVSRFSPYEWHTEEFEDGRETQSSESTNEFG
IFNSLWFSLGAFMQQGCDISPRSLSGRIVGGVWWFFTLIIISSYTANLAA
FLTVERMVSPIESAEDLSKQTEIAYGTLDSGSTKEFFRRSKIAVFDKMWT
YMRSAEPSVFVRTTAEGVARVRKSKGKYAYLLESTMNEYIEQRKPCDTMK
VGGNLDSKGYGIATPKGSSLRNAVNLAVLKLNEQGLLDKLKNKWWYDKGE
CGSGGGDSKEKTSALSLSNVAGVFYILVGGLGLAMLVALIEFCYKSRAEA
KRMKVAKNAQNINPSSSQNSQNFATYKEGYNVYGIESVKI
(M3 to CTD of GRIA2)
SEQ ID NO: 50
FGIFNSLWFSLGAFMQQGCDISPRSLSGRIVGGVWWFFTLIIISSYTANL
AAFLTVERMVSPIESAEDLSKQTEIAYGTLDSGSTKEFFRRSKIAVFDKM
WTYMRSAEPSVFVRTTAEGVARVRKSKGKYAYLLESTMNEYIEQRKPCDT
MKVGGNLDSKGYGIATPKGSSLRNAVNLAVLKLNEQGLLDKLKNKWWYDK
GECGSGGGDSKEKTSALSLSNVAGVFYILVGGLGLAMLVALIEFCYKSRA
EAKRMKVAKNAQNINPSSSQNSQNFATYKEGYNVYGIESVKI
(M4 to CTD of GRIA2)
SEQ ID NO: 51
VAGVFYILVGGLGLAMLVALIEFCYKSRAEAKRMKVAKNAQNINPSSSQN
SQNFATYKEGYNVYGIESVKI

In some embodiments, the sequence of GRIA2 is at least 70% identical to SEQ ID NO: 1 and still retains its ability to interact with the AP2-hippocalcin complex. For example, GRIA2 can be at least 70%, at least 75%, at least 80%, at least 85%, at least 90%, at least 95%, at least 99%, or 100% identical to the amino acid sequence of SEQ ID NO: 1. In one embodiment, the sequence of GRIA2 is at least 70% identical to any one of SEQ ID NOs: 49-51. For example, GRIA2 can be at least 70%, at least 75%, at least 80%, at least 85%, at least 90%, at least 95%, at least 99%, or 100% identical to the amino acid sequence of any one of SEQ ID NOs: 49-51.

As used herein, “GRIA2” or “glutamate ionotropic receptor AMPA type subunit 2” include those proteins that have at least one biological activity of GRIA2 from any mammalian species including but not limited to, human GRIA2, recombinant GRIA2, murine, bovine, porcine, and from other livestock or farm animals including but not limited to, chicken.

Accordingly, examples of GRIA2 include species and sequence variants thereof, but are not limited to one described herein. The GRIA2 can be the native, full-length protein or can be a truncated fragment or a sequence variant that retains at least a portion of the biological activity of the native protein.

In one embodiment, GRIA2 comprises a sequence corresponding to a protein found in nature. In another embodiment, the GRIA2 is a sequence variant, fragment, homolog, or a mimetics of a natural sequence that retains at least a portion of the biological activity of the corresponding native GRIA2.

Examples of the GRIA2 variants suitable here include those having sequences with homology to GRIA2 sequences, sequence fragments that are natural, such as from humans, non-human primates, mammals (including domestic animals), and non-natural sequence variants which retain at least a portion of the biologic activity or biological function of GRIA2 (e.g., the ability to interact with a AP2-hippocalcin complex). Non-mammalian GRIA2 sequences are also well known in the literature.

The term “GRIA2” encompasses GRIA2 polypeptides comprising one or more amino acid substitutions (e.g., particularly conservative modifications or conservative substitutions), additions or deletions. Exemplary substitutions (e.g., particularly conservative modifications or conservative substitutions) in a wide variety of amino acid positions in naturally-occurring GRIA2 have been described and are encompassed by the term “GRIA2 polypeptides.”

As used herein, the term “conservative sequence modifications” refers to amino acid modifications that do not significantly affect or alter (i) the binding of GRIA2 to the AP2-hippocalcin complex of the GRIA2 containing the amino acid sequence or (ii) the binding of AP2 to hippocalcin of the AP2 containing the amino acid sequence. Conservative amino acid substitutions are ones in which the amino acid residue is replaced with an amino acid residue having a similar side chain. Families of amino acid residues having similar side chains have been defined in the art.

Amino acid substitutions can be made, in some cases, by selecting substitutions that do not differ significantly in their effect on maintaining (a) the structure of the peptide backbone in the area of the substitution, (b) the charge or hydrophobicity of the molecule at the target sit; or (c) the bulk of the side chain. For example, naturally occurring residues can be divided into groups based on side-chain properties; (1) hydrophobic amino acids (norleucine, methionine, alanine, valine, leucine, and isoleucine); (2) neutral hydrophilic amino acids (cysteine, serine, threonine, asparagine, and glutamine,); (3) acidic amino acids (aspartic acid and glutamic acid); (4) basic amino acids (histidine, lysine, and arginine); (5) amino acids that influence chain orientation (glycine and proline); and (6) aromatic amino acids (tryptophan, tyrosine, and phenylalanine). Substitutions made within these groups can be considered conservative substitutions. Examples of substitutions include, without limitation, substitution of valine for alanine, lysine for arginine, glutamine for asparagine, glutamic acid for aspartic acid, serine for cysteine, asparagine for glutamine, aspartic acid for glutamic acid, proline for glycine, arginine for histidine, leucine for isoleucine, isoleucine for leucine, arginine for lysine, leucine for methionine, leucine for phenylalanine, glycine for proline, threonine for serine, serine for threonine, tyrosine for tryptophan, phenylalanine for tyrosine, and/or leucine for valine. Exemplary substitutions are shown in Table 2. Amino acid substitutions may be introduced into human GRIA2.

TABLE 2
Amino acid substitutions.

| Original Residue | Exemplary Substitutions |
|---|---|
| Ala (A) | Val; Leu; Ile |
| Arg (R) | Lys; Gln; Asn |
| Asn (N) | Gln; His; Asp, Lys; Arg |
| Asp (D) | Glu; Asn |
| Cys (C) | Ser; Ala |
| Gln (Q) | Asn; Glu |
| Glu (E) | Asp; Gln |
| Gly (G) | Ala |
| His (H) | Asn; Gln; Lys; Arg |
| Ile (I) | Leu; Val; Met; Ala; Phe; Norleucine |
| Leu (L) | Norleucine; Ile; Val; Met; Ala; Phe |
| Lys (K) | Arg; Gln; Asn |
| Met (M) | Leu; Phe; Ile |
| Phe (F) | Trp; Leu; Val; Ile; Ala; Tyr |
| Pro (P) | Ala |
| Ser (S) | Thr |
| Thr (T) | Val; Ser |
| Trp (W) | Tyr; Phe |
| Tyr (Y) | Trp; Phe; Thr; Ser |
| Val (V) | Ile; Leu; Met; Phe; Ala; Norleucine |

In some embodiments, the NLS C-fragment of a C-fusion protein described herein has an amino acid sequence as shown in Table 3.

TABLE 3
Exemplary NLS C-fragment and NLS N-
fragment sequences.

| | | N- | C- |
|---|---|---|---|
| | Original | fragment | fragment |
| Protein | Sequence | of NLS | of NLS |
| Xenopus | KRPAATKK/ | KRPAATKK | XXGQAKKK |
| laevis | AGQAKKKKLD | XX | KLD |
| nucleo- | (SEQ ID | (SEQ ID | (SEQ ID |
| plasmin | NO: 5) | NO: 3) | NO: 4) |
| Human | KRSAEGS/ | KRSAEGS | NPPKPLKK |
| RB | NPPKPLKKLR | (SEQ ID | LR |
| | (SEQ ID | NO: 7) | (SEQ ID |
| | NO: 6) | | NO: 8) |
| Human | KRALPNNTS | KRALPNNT | PQPKKKP |
| p53 | SS/PQPKKKP | SSS | (SEQ ID |
| | (SEQ ID | (SEQ ID | NO: 11) |
| | NO: 9) | NO: 10) | |

Note:
X can be any amino acid. The “/” indicates where the sequence can be split.

Xenopus laevis nucleoplasmin is an abundant protein found in the nucleus of Xenopus laevis oocytes. Nucleoplasmin is a molecular chaperone that binds histones H2A and H2B and transfers them to DNA.

Human RB (retinoblastoma) is a protein that inhibits cell cycle progression from the G1 to the S phase of the cell cycle. RB is localized to the cell nucleus.

Human p53 is a nuclear transcription factor. p53 transactivates different target genes involved in the induction of cell cycle arrest and/or apoptosis.

As used herein, the terms “NLS C-fragment,” “NLS_{c} fragment,” and “c-NLS fragment,” are interchangeable. Additionally, the terms, “NLS N-fragment,” “NLS_{N} fragment,” and “N-NLS fragment,” are interchangeable.

In one embodiment, the C-fusion protein comprises CFN tripeptide sequence (SEQ ID NO: 18; CFN). The CFN tripeptide sequence is required for efficient protein splicing of the inteins of the biosensor proteins described herein.

In some embodiments, the C-fusion protein comprises a C-intein as shown in Table 1.

The C-fusion protein described herein further comprises a reporter molecule. In one embodiment, the reporter molecule is a fluorescent protein. In some embodiments, the fluorescent protein is a green fluorescent protein (GFP), enhanced green fluorescent protein (eGFP), GFPxm, or a variant thereof such as those described in U.S. Pat. No. 5,625,048 or U.S. Pat. No. 7,091,317. In some embodiments, the fluorescent protein comprises yellow fluorescent protein (YFP), mCherry, red fluorescent protein (RFP), blue fluorescent protein (BFP), cyan fluorescent protein (CFP), cerulean, DsRed, mBanana, mHoneydew, aquamarine, or any other fluorescent protein as known in the art. The reporter molecule translocates to the nucleus following heterodimerization of the C-fusion and N-fusion proteins in response to the presence of hippocalcin.

In some embodiments, the C-fusion protein described herein further comprises a 5′ long terminal repeat (LTR), HIV-1 psi packaging signal (ψ), RPE (Rev responsive element), PPT (polypurine tract), elongation factor 1 α (EF1α), a woodchuck hepatitis virus post-transcriptional regulatory element (WPRE) sequence, and a 3′ long terminal repeat (LTR).

WPRE sequences are known in the art. In some embodiments, the C-fusion protein comprises any type of WPRE. Examples of WPRE include, but are not limited to WPRE2, WPRE3, and WPRE6. In some embodiments, a WPRE sequence described in Zanta-Boussif et al, Validation of a mutated PRE sequence allowing high and sustained transgene expression while abrogating WHV-X protein synthesis: application to the gene therapy of WAS. Gene Ther. 2009 May; 16(5):605-19 is used.

The HIV-1 Rev response element (RRE) is a ˜350 nucleotide, highly structured, cis-acting RNA element essential for viral replication. It is located in the env coding region of the viral genome and is extremely well conserved across different HIV-1 isolates. All lentiviruses encode a regulatory protein Rev that is essential for post-transcriptional transport of the unspliced and incompletely spliced viral mRNAs from nuclei to cytoplasm. The Rev protein acts via binding to an RNA structural element known as the Rev responsive element (RRE).

The central polypurine tract (PPT) participates in lentiviral reverse transcription by initiating the synthesis of a downstream plus strand cDNA. The PPT is conversed in most retroviruses. In one embodiment, the PPT comprises the nucleic acid sequence of SEQ ID NO: 24 (AAAAGAAAAGGGGGG).

Long terminal repeats (LTRs) are pairs of identical sequence of DNA that are hundreds of base pairs long. An LTR is present on either side of a viral genome. LTRs comprise cis-acting elements that are required for RNA synthesis and function as the initiation site for transcription of the viral genome. LTRs comprise three distinct regions: U3 (unique 3′ end), R (repeated), and U5 (unique 5′ end). Elements present in U3 help guide direct binding of RNA polymerase II to DNA templates.

In one embodiment, the C-fusion protein comprises the amino acid sequence of SEQ ID NO: 20.

(GRIA2-C-intein-NLS_{C} fragment-EGFP)
SEQ ID NO: 20
MQKIMHISVLLSPVLWGLIFGVSSNSIQIGGLFPRGADQEYSAFRVG
MVQFSTSEFRLTPHIDNLEVANSFAVTNAFCSQFSRGVYAIFGFYDK
KSVNTITSFCGTLHVSFITPSFPTDGTHPFVIQMRPDLKGALLSLIE
YYQWDKFAYLYDSDRGLSTLQAVLDSAAEKKWQVTAINVGNINNDKK
DEMYRSLFQDLELKKERRVILDCERDKVNDIVDQVITIGKHVKGYHY
IIANLGFTDGDLLKIQFGGANVSGFQIVDYDDSLVSKFIERWSTLEE
KEYPGAHTTTIKYTSALTYDAVQVMTEAFRNLRKQRIEISRRGNAGD
CLANPAVPWGQGVEIERALKQVQVEGLSGNIKFDQNGKRINYTINIM
ELKTNGPRKIGYWSEVDKMVVTLTELPSGNDTSGLENKTVVVTTILE
SPYVMMKKNHEMLEGNERYEGYCVDLAAEIAKHCGFKYKLTIVGDGK
YGARDADTKIWNGMVGELVYGKADIAIAPLTITLVREEVIDFSKPFM
SLGISIMIKKPQKSKPGVFSFLDPLAYEIWMCIVFAYIGVSVVLFLV
SRFSPYEWHTEEFEDGRETQSSESTNEFGIFNSLWFSLGAFMQQGCD
ISPRSLSGRIVGGVWWFFTLIIISSYTANLAAFLTVERMVSPIESAE
DLSKQTEIAYGTLDSGSTKEFFRRSKIAVFDKMWTYMRSAEPSVFVR
TTAEGVARVRKSKGKYAYLLESTMNEYIEQRKPCDTMKVGGNLDSKG
YGIATPKGSSLRNAVNLAVLKLNEQGLLDKLKNKWWYDKGECGSGGG
DSKEKTSALSLSNVAGVFYILVGGLGLAMLVALIEFCYKSRAEAKRM
KVAKNAQNINPSSSQNSQNFATYKEGYNVYGIESVKITRMVLLNVLS
KCAGSKKFRPAPAAAFARECRGFYFELQELKEDDYYGITLSDDSDHQ
FLLANQVVVHNCFNVDGQAKKKKLDMVSKGEELFTGVVPILVELDGD
VNGHKFSVSGEGEGDATYGKLTLKFICTTGKLPVPWPTLVTTLTYGV
QCFSRYPDHMKQHDFFKSAMPEGYVQERTIFFKDDGNYKTRAEVKFE
GDTLVNRIELKGIDFKEDGNILGHKLEYNYNSHNVYIMADKQKNGIK
VNFKIRHNIEDGSVQLADHYQQNTPIGDGPVLLPDNHYLSTQSALSK
DPNEKRDHMVLLEFVTAAGITLGMDELYK

Note that the GRIA2 sequence is in bold (SEQ ID NO: 1). The sequence of C-intein is underlined (SEQ ID NO: 13). The sequence of NLSc is in italics (SEQ ID NO: 4). The EGFP sequence is in bold and underlined (SEQ ID NO: 52).

In one embodiment, the C-fusion protein is encoded by a vector such as a lentiviral vector. In one embodiment, the sequence of the lentiviral vector encoding the N-fusion protein comprises the sequence of SEQ ID NO: 22.

(GRIA2-C-intein-c-NLS fragment-EGFP)
SEQ ID NO: 22
aatgtagtcttatgcaatactcttgtagtcttgcaacatggtaacgatgagttagcaacatgccttacaaggagagaaaaagcaccgtgcatgccgatt
ggtggaagtaaggtggtacgatcgtgccttattaggaaggcaacagacgggtctgacatggattggacgaaccactgaattgccgcattgcagagatat
tgtatttaagtgcctagctcgatacataaacgggtctctctggttagaccagatctgagcctgggagctctctggctaactagggaacccactgcttaa
gcctcaataaagcttgccttgagtgcttcaagtagtgtgtgcccgtctgttgtgtgactctggtaactagagatccctcagacccttttagtcagtgtg
gaaaatctctagcagtggcgcccgaacagggacttgaaagcgaaagggaaaccagaggagctctctcgacgcaggactcggcttgctgaagcgcgcacg
gcaagaggcgaggggcggcgactggtgagtacgccaaaaattttgactagcggaggctagaaggagagagatgggtgcgagagcgtcagtattaagcgg
gggagaattagatcgcgatgggaaaaaattcggttaaggccagggggaaagaaaaaatataaattaaaacatatagtatgggcaagcagggagctag
aacgattcgcagttaatcctggcctgttagaaacatcagaaggctgtagacaaatactgggacagctacaaccatcccttcagacaggatcagaagaa
cttagatcattatataatacagtagcaaccctctattgtgtgcatcaaaggatagagataaaagacaccaaggaagctttagacaagatagaggaagag
caaaacaaaagtaagaccaccgcacagcaagcggccgctgatcttcagacctggaggaggagatatgagggacaattggagaagtgaattatataaa
tataaagtagtaaaaattgaaccattaggagtagcacccaccaaggcaaagagaagagtggtgcagagagaaaaaagagcagtgggaataggagct
ttgttccttgggttcttgggagcagcaggaagcactatgggcgcagcgtcaatgacgctgacggtacaggccagacaattattgtctggtatagtgcag
cagcagaacaatttgctgagggctattgaggcgcaacagcatctgttgcaactcacagtctggggcatcaagcagctccaggcaagaatcctggctgtg
gaaagatacctaaaggatcaacagctcctggggatttggggttgctctggaaaactcatttgcaccactgctgtgccttggaatgctagttggagtaat
aaatctctggaacagatttggaatcacacgacctggatggagtgggacagagaaattaacaattacacaagcttaatacactccttaattgaagaatcg
caaaaccagcaagaaaagaatgaacaagaattattggaattagataaatgggcaagtttgtggaattggtttaacataacaaattggctgtggtatata
aaattattcataatgatagtaggaggcttggtaggtttaagaatagtttttgctgtactttctatagtgaatagagttaggcagggatattcaccatta
tcgtttcagacccacctcccaaccccgaggggacccgacaggcccgaaggaatagaagaagaaggtggagagagagacagagacagatccattcgatta
gtgaacggatctcgacggtatcgctagcttttaaaagaaaaggggggattggggggtacagtgcaggggaaagaatagtagacataatagcaacagaca
tacaaactaaagaattacaaaaacaaattacaaaaattcaaaattttactagtgattatcggatcaactttgtatagaaaagttgggctccggtgcccg
tcagtgggcagagcgcacatcgcccacagtccccgagaagttggggggaggggtcggcaattgaaccggtgcctagagaaggtggcgcggggtaaact
gggaaagtgatgtcgtgtactggctccgcctttttcccgagggtgggggagaaccgtatataagtgcagtagtcgccgtgaacgttctttttcgcaacg
ggtttgccgccagaacacaggtaagtgccgtgtgtggttcccgcgggcctggcctctttacgggttatggcccttgcgtgccttgaattacttccacct
ggctgcagtacgtgattcttgatcccgagcttcgggttggaagtgggtgggagagttcgaggccttgcgcttaaggagccccttcgcctcgtgcttgag
ttgaggcctggcctgggcgctggggccgccgcgtgcgaatctggtggcaccttcgcgcctgtctcgctgctttcgataagtctctagccatttaaaatt
tttgatgacctgctgcgacgctttttttctggcaagatagtcttgtaaatgcgggccaagatctgcacactggtatttcggtttttggggccgcgggcg
gcgacggggcccgtgcgtcccagcgcacatgttcggcgaggcggggcctgcgagcgcggccaccgagaatcggacgggggtagtctcaagctggccggc
ctgctctggtgcctggtctcgcgccgccgtgtatcgccccgccctgggcggcaaggctggcccggtcggcaccagttgcgtgagcggaaagatggccgc
ttcccggccctgctgcagggagctcaaaatggaggacgcggcgctcgggagagcggggggtgagtcacccacacaaaggaaaagggcctttccgtcctc
agccgtcgcttcatgtgactccacggagtaccgggcgccgtccaggcacctcgattagttctcgagcttttggagtacgtcgtctttaggttgggggga
ggggttttatgcgatggagtttccccacactgagtgggtggagactgaagttaggccagcttggcacttgatgtaattctccttggaatttgccctttt
tgagtttggatcttggttcattctcaagcctcagacagtggttcaaagtttttttcttccatttcaggtgtcgtgacaagtttgtacaaaaaagcaggc
tgccaccatgcaaaagattatgcatatttctgtcctcctttctcctgttttatggggactgatttttggtgtctcttctaacagcatacagataggggg
gctatttcctaggggcgccgatcaagaatacagtgcatttcgagtagggatggttcagttttccacttcggagttcagactgacaccccacatcgacaa
tttggaggtggcaaacagcttcgcagtcactaatgctttctgctcccagttttcgagaggagtctatgctatttttggattttatgacaagaagtctgt
aaataccatcacatcattttgcggaacactccacgtctccttcatcactcccagcttcccaacagatggcacacatccatttgtcattcagatgagacc
cgacctcaaaggagctctccttagcttgattgaatactatcaatgggacaagtttgcatacctctatgacagtgacagaggcttatcaacactgcaagc
tgtgctggattctgctgctgaaaagaaatggcaagtgactgctatcaatgtgggaaacattaacaatgacaagaaagatgagatgtaccgatcactttt
tcaagatctggagttaaaaaaggaacggcgtgtaattctggactgtgaaagggataaagtaaacgacattgtagaccaggttattaccattggaaaaca
tgttaaagggtaccactacatcattgcaaatctgggatttactgatggagacctattaaaaatccagtttggaggtgcaaatgtctctggatttcagat
agtggactatgatgattcgttggtatctaaatttatagaaagatggtcaacactggaagaaaaagaataccctggagctcacacaacaacaattaagta
tacttctgctctgacctatgatgccgttcaagtgatgactgaagccttccgcaacctaaggaagcaaagaattgaaatctcccgaagggggaatgcagg
agactgtctggcaaacccagcagtgccctggggacaaggtgtagaaatagaaagggccctcaaacaggttcaggttgaaggtctctcaggaaatataaa
gtttgaccagaatggaaaaagaataaactatacaattaacatcatggagctcaaaactaatgggccccggaagattggctactggagtgaagtggacaa
aatggttgttacccttactgagctcccttctggaaatgacacctctgggcttgagaataagactgttgttgtcaccacaattttggaatctccgtatgt
tatgatgaagaaaaatcatgaaatgcttgaaggcaatgagcgctatgagggctactgtgttgacctggctgcagaaatcgccaaacattgtgggttcaa
gtacaagttgacaattgttggtgatggcaagtatggggccagggatgcagacacgaaaatttggaatgggatggttggagaacttgtatatgggaaagc
tgatattgcaattgctccattaactattacccttgtgagagaagaggtgattgacttctcaaagcccttcatgagcctcgggatatctatcatgatcaa
gaagcctcagaagtccaaaccaggagtgttttcctttcttgatcctttagcctatgagatctggatgtgcattgtttttgcctacattggggtcagtgt
agttttattcctggtcagcagatttagcccctacgagtggcacactgaggagtttgaagatggaagagaaacacaaagtagtgaatcaactaatgaatt
tgggatttttaatagtctctggttttccttgggtgcctttatgcagcaaggatgcgatatttcgccaagatccctctctgggcgcattgttggaggtgt
gtggtggttctttaccctgatcataatctcctcctacacggctaacttagctgccttcctgactgtagagaggatggtgtctcccatcgaaagtgctga
ggatctttctaagcaaacagaaattgcttatggaacattagactctggctccactaaagagtttttcaggagatctaaaattgcagtgtttgataaaat
gtggacctacatgcggagtgcggagccctctgtgtttgtgaggactacggccgaaggggtggctagagtgcggaagtccaaagggaaatatgcctactt
gttggagtccacgatgaacgagtacattgagcaaaggaagccttgcgacaccatgaaagttggtggaaacctggattccaaaggctatggcatcgcaac
acctaaaggatcctcattaagaaatgcggttaacctcgcagtactaaaactgaatgaacaaggcctgttggacaaattgaaaaacaaatggtggtacga
caaaggagagtgcggcagcgggggaggtgattccaaggaaaagaccagtgccctcagtctgagcaacgttgctggagtattctacatccttgtcggggg
ccttggtttggcaatgctggtggctttgattgagttctgttacaagtcaagggccgaggcgaaacgaatgaaggtggcaaagaatgcacagaatattaa
cccatcttcctcgcagaattcacagaattttgcaacttataaggaaggttacaacgtatatggcatcgaaagtgttaaaattacgcgtatggttttgct
taacgttctttcgaagtgtgccggctctaaaaaattcaggcctgctcccgccgctgcttttgcacgtgagtgccgcggattttatttcgagttacaaga
attgaaggaagacgattattatgggattactttatctgatgattctgatcatcagtttttgcttgcaaaccaggttgtcgtccataactgtttcaacgt
cgacggccaggcgaaaaagaagaaactggatatggtgagcaagggcgaggagctgttcaccggggtggtgcccatcctggtcgagctggacggcgacgt
aaacggccacaagttcagcgtgtccggcgagggcgagggcgatgccacctacggcaagctgaccctgaagttcatctgcaccaccggcaagctgcccgt
gccctggcccaccctcgtgaccaccctgacctacggcgtgcagtgcttcagccgctaccccgaccacatgaagcagcacgacttcttcaagtccgccat
gcccgaaggctacgtccaggagcgcaccatcttcttcaaggacgacggcaactacaagacccgcgccgaggtgaagttcgagggcgacaccctggtgaa
ccgcatcgagctgaagggcatcgacttcaaggaggacggcaacatcctggggcacaagctggagtacaactacaacagccacaacgtctatatcatggc
cgacaagcagaagaacggcatcaaggtgaacttcaagatccgccacaacatcgaggacggcagcgtgcagctcgccgaccactaccagcagaacacccc
catcggcgacggccccgtgctgctgcccgacaaccactacctgagcacccagtccgccctgagcaaagaccccaacgagaagcgcgatcacatggtcct
gctggagttcgtgaccgccgccgggatcactctcggcatggacgagctgtacaagtaaacccagctttcttgtacaaagtggtgataatcgaattccga
taatcaacctctggattacaaaatttgtgaaagattgactggtattcttaactatgttgctccttttacgctatgtggatacgctgctttaatgccttt
gtatcatgctattgcttcccgtatggctttcattttctcctccttgtataaatcctggttgctgtctctttatgaggagttgtggcccgttgtcaggca
acgtggcgtggtgtgcactgtgtttgctgacgcaacccccactggttggggcattgccaccacctgtcagctcctttccgggactttcgctttccccct
ccctattgccacggcggaactcatcgccgcctgccttgcccgctgctggacaggggctcggctgttgggcactgacaattccgtggtgttgtcggggaa
gctgacgtcctttccatggctgctcgcctgtgttgccacctggattctgcgcgggacgtccttctgctacgtcccttcggccctcaatccagcggacct
tccttcccgcggcctgctgccggctctgcggcctcttccgcgtcttcgccttcgccctcagacgagtcggatctccctttgggccgcctccccgcatcg
ggaattcccgcggttcgaattctaccgggtaggggaggcgcttttcccaaggcagtctggagcatgcgctttagcagccccgctgggcacttggcgcta
cacaagtggcctctggcctcgcacacattccacatccaccggtaggcgccaaccggctccgttctttggtggccccttcgcgccaccttctactcctcc
cctagtcaggaagttcccccccgccccgcagctcgcgtcgtgcaggacgtgacaaatggaagtagcacgtctcactagtctcgtgcagatggacagcac
cgctgagcaatggaagcgggtaggcctttggggcagcggccaatagcagctttgctccttcgctttctgggctcagaggctgggaaggggtgggtccgg
gggcgggctcaggggcgggctcaggggcggggcgggcgcccgaaggtcctccggaggcccggcattctgcacgcttcaaaagcgcacgtctgccgcgct
gttctcctcttcctcatctccgggcctttcgacctcacgtggccaccatgaccgagtacaagcccacggtgcgcctcgccacccgcgacgacgtcccca
gggccgtacgcaccctcgccgccgcgttcgccgactaccccgccacgcgccacaccgtcgatccggaccgccacatcgagcgggtcaccgagctgcaag
aactcttcctcacgcgcgtcgggctcgacatcggcaaggtgtgggtcgcggacgacggcgccgcggtggcggtctggaccacgccggagagcgtcgaag
cgggggcggtgttcgccgagatcggcccgcgcatggccgagttgagcggttcccggctggccgcgcagcaacagatggaaggcctcctggcgccgcacc
ggcccaaggagcccgcgtggttcctggccaccgtcggcgtctcgcccgaccaccagggcaagggtctgggcagcgccgtcgtgctccccggagtggagg
cggccgagcgcgccggggtgcccgccttcctggagacctccgcgccccgcaacctccccttctacgagcggctcggcttcaccgtcaccgccgacgtcg
aggtgcccgaaggaccgcgcacctggtgcatgacccgcaagcccggtgcctgaggtacctttaagaccaatgacttacaaggcagctgtagatcttagc
cactttttaaaagaaaaggggggactggaagggctaattcactcccaacgaagacaagatctgctttttgcttgtactgggtctctctggttagaccag
atctgagcctgggagctctctggctaactagggaacccactgcttaagcctcaataaagcttgccttgagtgcttcaagtagtgtgtgcccgtctgttg
tgtgactctggtaactagagatccctcagacccttttagtcagtgtggaaaatctctagcagtagtagttcatgtcatcttattattcagtatttataa
cttgcaaagaaatgaatatcagagagtgagaggaacttgtttattgcagcttataatggttacaaataaagcaatagcatcacaaatttcacaaataaa
gcatttttttcactgcattctagttgtggtttgtccaaactcatcaatgtatcttatcatgtctggctctagctatcccgcccctaactccgcccatcc
cgcccctaactccgcccagttccgcccattctccgccccatggctgactaattttttttatttatgcagaggccgaggccgcctcggcctctgagctat
tccagaagtagtgaggaggcttttttggaggcctagggacgtacccaattcgccctatagtgagtcgtattacgcgcgctcactggccgtcgttttaca
acgtcgtgactgggaaaaccctggcgttacccaacttaatcgccttgcagcacatccccctttcgccagctggcgtaatagcgaagaggcccgcaccga
tcgcccttcccaacagttgcgcagcctgaatggcgaatgggacgcgccctgtagcggcgcattaagcgcggcgggtgtggtggttacgcgcagcgtgac
cgctacacttgccagcgccctagcgcccgctcctttcgctttcttcccttcctttctcgccacgttcgccggctttccccgtcaagctctaaatcgggg
gctccctttagggttccgatttagtgctttacggcacctcgaccccaaaaaacttgattagggtgatggttcacgtagtgggccatcgccctgatagac
ggtttttcgccctttgacgttggagtccacgttctttaatagtggactcttgttccaaactggaacaacactcaaccctatctcggtctattcttttga
tttataagggattttgccgatttcggcctattggttaaaaaatgagctgatttaacaaaaatttaacgcgaattttaacaaaatattaacgcttacaat
ttaggtggcacttttcggggaaatgtgcgcggaacccctatttgtttatttttctaaatacattcaaatatgtatccgctcatgagacaataaccctga
taaatgcttcaataatattgaaaaaggaagagtatgagtattcaacatttccgtgtcgcccttattcccttttttgcggcattttgccttcctgttttt
gctcacccagaaacgctggtgaaagtaaaagatgctgaagatcagttgggtgcacgagtgggttacatcgaactggatctcaacagcggtaagatcctt
gagagttttcgccccgaagaacgttttccaatgatgagcacttttaaagttctgctatgtggcgcggtattatcccgtattgacgccgggcaagagcaa
ctcggtcgccgcatacactattctcagaatgacttggttgagtactcaccagtcacagaaaagcatcttacggatggcatgacagtaagagaattatgc
agtgctgccataaccatgagtgataacactgcggccaacttacttctgacaacgatcggaggaccgaaggagctaaccgcttttttgcacaacatgggg
gatcatgtaactcgccttgatcgttgggaaccggagctgaatgaagccataccaaacgacgagcgtgacaccacgatgcctgtagcaatggcaacaacg
ttgcgcaaactattaactggcgaactacttactctagcttcccggcaacaattaatagactggatggaggcggataaagttgcaggaccacttctgcgc
tcggcccttccggctggctggtttattgctgataaatctggagccggtgagcgtgggtctcgcggtatcattgcagcactggggccagatggtaagccc
tcccgtatcgtagttatctacacgacggggagtcaggcaactatggatgaacgaaatagacagatcgctgagataggtgcctcactgattaagcattgg
taactgtcagaccaagtttactcatatatactttagattgatttaaaacttcatttttaatttaaaaggatctaggtgaagatcctttttgataatctc
atgaccaaaatcccttaacgtgagttttcgttccactgagcgtcagaccccgtagaaaagatcaaaggatcttcttgagatcctttttttctgcgcgta
atctgctgcttgcaaacaaaaaaaccaccgctaccagcggtggtttgtttgccggatcaagagctaccaactctttttccgaaggtaactggcttcagc
agagcgcagataccaaatactgttcttctagtgtagccgtagttaggccaccacttcaagaactctgtagcaccgcctacatacctcgctctgctaatc
ctgttaccagtggctgctgccagtggcgataagtcgtgtcttaccgggttggactcaagacgatagttaccggataaggcgcagcggtcgggctgaacg
gggggttcgtgcacacagcccagcttggagcgaacgacctacaccgaactgagatacctacagcgtgagctatgagaaagcgccacgcttcccgaagag
agaaaggcggacaggtatccggtaagcggcagggtcggaacaggagagcgcacgagggagcttccagggggaaacgcctggtatctttatagtcctgtc
gggtttcgccacctctgacttgagcgtcgatttttgtgatgctcgtcaggggggcggagcctatggaaaaacgccagcaacgcggcctttttacggttc
ctggccttttgctggccttttgctcacatgttctttcctgcgttatcccctgattctgtggataaccgtattaccgcctttgagtgagctgataccgct
cgccgcagccgaacgaccgagcgcagcgagtcagtgagcgaggaagcggaagagcgcccaatacgcaaaccgcctctccccgcgcgttggccgattcat
taatgcagctggcacgacaggtttcccgactggaaagcgggcagtgagcgcaacgcaattaatgtgagttagctcactcattaggcaccccaggcttta
cactttatgcttccggctcgtatgttgtgtggaattgtgagcggataacaatttcacacaggaaacagctatgaccatgattacgccaagcgcgcaatt
aaccctcactaaagggaacaaaagctggagctgcaagctt

### 4B. N-Fusion Proteins

In one embodiment, the N-fusion protein comprises an NLS N-fragment, an N-intein, and an Adaptor Protein Complex 2 (AP2) (from 5′ to 3′ orientation). The components of the N-fusion protein are operatively linked together.

An exemplary sequence of AP2 is shown below as SEQ ID NO: 2.

SEQ ID NO: 2 (AP2):
MTDSKYFTTNKKGEIFELKAELNNEKKEKRKEAVKKVIAAMTVGKD
VSSLFPDVVNCMQTDNLELKKLVYLYLMNYAKSQPDMAIMAVNSFV
KDCEDPNPLIRALAVRTMGCIRVDKITEYLCEPLRKCLKDEDPYVR
KTAAVCVAKLHDINAQMVEDQGFLDSLRDLIADSNPMVVANAVAAL
SEISESHPNSNLLDLNPQNINKLLTALNECTEWGQIFILDCLSNYN
PKDDREAQSICERVTPRLSHANSAVVLSAVKVLMKFPELLPKDSDY
YNMLLKKLAPPLVTLLSGEPEVQYVALRNINLIVQKRPEILKQEIK
VFFVKYNDPIYVKLEKLDIMIRLASQANIAQVLAELKEYATEVDVD
FVRKAVRAIGRCAIKVEQSAERCVSTLLDLIQTKVNYVVQEAIVVI
RDIFRKYPNKYESIIATLCENLDSLDEPDARAAMIWIVGEYAERID
NADELLESFLEGFHDESTQVQLTLLTAIVKLFLKKPSETQELVQQV
LSLATQDSDNPDLRDRGYIYWRLLSTDPVTAKEVVLSEKPLISEET
DLIEPTLLDELICHIGSLASVYHKPPNAFVEGSHGIHRKHLPIHHG
STDAGDSPVGTTTATNLEQPQVIPSQGDLLGDLLNLDLGPPVNVPQ
VSSMQMGAVDLLGGGLDSLLGSDLGGGIGGSPAVGQSFIPSSVPAT
FAPSPTPAVVSSGLNDLFELSTGIGMAPGGYVAPKAVWLPAVKAKG
LEISGTFTHRQGHIYMEMNFTNKALQHMTDFAIQFNKNSFGVIPST
PLAIHTPLMPNQSIDVSLPLNTLGPVMKMEPLNNLQVAVKNNIDVF
YFSCLIPLNVLFVEDGKMERQVFLATWKDIPNENELQFQIKECHLN
ADTVSSKLQNNNVYTIAKRNVEGQDMLYQSLKLTNGIWILAELRIQ
PGNPNYTLSLKCRAPEVSQYIYQVYDSILKN

In some embodiments, the sequence of AP2 is at least 70% identical to SEQ ID NO: 2 and still retains its ability to bind to hippocalcin. For example, the AP2 can be at least 70%, at least 75%, at least 80%, at least 85%, at least 90%, at least 95%, at least 99%, or 100% identical to the amino acid sequence of SEQ ID NO: 2.

As used herein, “AP2” or “Adaptor Protein Complex 2” include those proteins that have at least one biological activity of AP2 from any mammalian species including but not limited to, human AP2, recombinant AP2, murine, bovine, porcine, and from other livestock or farm animals including but not limited to, chicken.

In one embodiment, AP2 comprises a sequence corresponding to a protein found in nature. In another embodiment, the AP2 is a sequence variant, fragment, homolog, or a mimetics of a natural sequence that retains at least a portion of the biological activity of the corresponding native AP2.

Examples of the AP2 variants suitable here include those having sequences with homology to AP2 sequences, sequence fragments that are natural, such as from humans, non-human primates, mammals (including domestic animals), and non-natural sequence variants which retain at least a portion of the biologic activity or biological function of AP2 (e.g., the ability to bind to hippocalcin). Non-mammalian AP2 sequences are also well known in the literature.

The term “AP2” encompasses AP2 polypeptides comprising one or more amino acid substitutions (e.g., particularly conservative modifications or conservative substitutions), additions or deletions. Exemplary substitutions (e.g., particularly conservative modifications or conservative substitutions) in a wide variety of amino acid positions in naturally-occurring AP2 have been described and are encompassed by the term “AP2 polypeptides.” Exemplary amino acid substitutions are shown in Table 2.

In some embodiments, the NLS N-fragment of a N-fusion protein described herein has an amino acid sequence as shown in Table 3.

In some embodiments, the N-fusion protein comprises a N-intein as shown in Table 1.

In one embodiment, the N-fusion protein comprises a P2A sequence. P2A is a self-cleaving peptide that is from picornavirus, which mediates ribosome-skipping events. In one embodiment, P2A comprises the amino acid sequence of SEQ ID NO: 23.

(P2A)
SEQ ID NO: 23
GSGATNFSLLKQAGDVEENPGP

For example, in the absence of P2A, the fluorescent protein (such as mCherry) remains at the C terminus of the N-fusion protein. The use of P2A is to minimize the interference of the fluorescent protein (e.g., mCherry) on the interaction between AP2 of the N-fusion protein and hippocalcin.

In one embodiment, the N-fusion protein comprises a fluorescent protein inserted at the C-terminus of AP2. This fluorescent protein can be used to verify that expression of the N-fusion protein in cells of interest. In some embodiments, the fluorescent protein is a different fluorescent protein than the reporter molecule of the C-fusion protein. In one embodiment, the fluorescent protein is mCherry. In some embodiments, the fluorescent protein is a green fluorescent protein (GFP), enhanced green fluorescent protein (eGFP), GFPxm, or a variant thereof such as those described in U.S. Pat. No. 5,625,048 or U.S. Pat. No. 7,091,317. In some embodiments, the fluorescent protein comprises yellow fluorescent protein (YFP), mCherry, red fluorescent protein (RFP), blue fluorescent protein (BFP), cyan fluorescent protein (CFP), cerulean, DsRed, mBanana, mHoneydew, aquamarine, or any other fluorescent protein as known in the art.

In some embodiments, the N-fusion protein further comprises 5′ long terminal repeat (LTR), HIV-1 psi packaging signal (yv), RPE (Rev responsive element), PPT (polypurine tract), hPGK (human phosphoglycerate kinase promoter), a woodchuck hepatitis virus post-transcriptional regulatory element (WPRE) sequence, and a 3′ long terminal repeat (LTR). In some embodiments, the N-fusion protein comprises any type of WPRE as discussed above for the C-fusion protein.

In one embodiment, the N-fusion protein comprises the amino acid sequence of SEQ ID NO: 19.

SEQ ID NO: 19 (NLS_{N}-N-intein-AP2):
MKRPAATKKHMCFAKGTNVLMADGSIECIENIEVGNKVMGKDGRPR
EVIKLPRGRETMYSVVQKSQHRAHKSDSSREVPELLKFTCNATHEL
VVRTPRSVRRLSRTIKGVEYFEVITFEMGQKKAPDGRIVELVKEVS
KSYPISEGPERANELVESYRKASNKAYFEWTIEARDLSLLGSHVRK
ATYQTYAPILYSRTDSKYFTTNKKGEIFELKAELNNEKKEKRKEAV
KKVIAAMTVGKDVSSLFPDVVNCMQTDNLELKKLVYLYLMNYAKSQ
PDMAIMAVNSFVKDCEDPNPLIRALAVRTMGCIRVDKITEYLCEPL
RKCLKDEDPYVRKTAAVCVAKLHDINAQMVEDQGFLDSLRDLIADS
NPMVVANAVAALSEISESHPNSNLLDLNPQNINKLLTALNECTEWG
QIFILDCLSNYNPKDDREAQSICERVTPRLSHANSAVVLSAVKVLM
KFPELLPKDSDYYNMLLKKLAPPLVTLLSGEPEVQYVALRNINLIV
QKRPEILKQEIKVFFVKYNDPIYVKLEKLDIMIRLASQANIAQVLA
ELKEYATEVDVDFVRKAVRAIGRCAIKVEQSAERCVSTLLDLIQTK
VNYVVQEAIVVIRDIFRKYPNKYESIIATLCENLDSLDEPDARAAM
IWIVGEYAERIDNADELLESFLEGFHDESTQVQLTLLTAIVKLFLK
KPSETQELVQQVLSLATQDSDNPDLRDRGYIYWRLLSTDPVTAKEV
VLSEKPLISEETDLIEPTLLDELICHIGSLASVYHKPPNAFVEGSH
GIHRKHLPIHHGSTDAGDSPVGTTTATNLEQPQVIPSQGDLLGDLL
NLDLGPPVNVPQVSSMQMGAVDLLGGGLDSLLGSDLGGGIGGSPAV
GQSFIPSSVPATFAPSPTPAVVSSGLNDLFELSTGIGMAPGGYVAP
KAVWLPAVKAKGLEISGTFTHRQGHIYMEMNFTNKALQHMTDFAIQ
FNKNSFGVIPSTPLAIHTPLMPNQSIDVSLPLNTLGPVMKMEPLNN
LQVAVKNNIDVFYFSCLIPLNVLFVEDGKMERQVFLATWKDIPNEN
ELQFQIKECHLNADTVSSKLQNNNVYTIAKRNVEGQDMLYQSLKLT
NGIWILAELRIQPGNPNYTLSLKCRAPEVSQYIYQVYDSILKNGSG
ATNFSLLKQAGDVEENPGPMVSKGEEDNMAIIKEFMRFKVHMEGSV
NGHEFEIEGEGEGRPYEGTQTAKLKVTKGGPLPFAWDILSPQFMYG
SKAYVKHPADIPDYLKLSFPEGFKWERVMNFEDGGVVTVTQDSSLQ
DGEFIYKVKLRGTNFPSDGPVMQKKTMGWEASSERMYPEDGALKGE
IKQRLKLKDGGHYDAEVKTTYKAKKPVQLPGAYNVNIKLDITSHNE
DYTIVEQYERAEGRHSTGGMDELYK

Note that the sequence of NLSN is in bold (SEQ ID NO: 3). The N-intein sequence is underlined (SEQ ID NO: 12). The sequence of AP2 (SEQ ID NO: 2) is in italics.

In one embodiment, the N-fusion protein is encoded by a vector such as a lentiviral vector. In one embodiment, the sequence of the lentiviral vector encoding the N-fusion protein comprises the sequence of SEQ ID NO: 21.

(n-NLS fragment-N-intein-AP2 P2A mCherry)
SEQ ID NO: 21
aatgtagtcttatgcaatactcttgtagtcttgcaacatggtaacgatgagttagcaacatgccttacaaggagagaaaaagcaccgtgcatgccgatt
ggtggaagtaaggtggtacgatcgtgccttattaggaaggcaacagacgggtctgacatggattggacgaaccactgaattgccgcattgcagag
atattgtatttaagtgcctagctcgatacataaacgggtctctctggttagaccagatctgagcctgggagctctctggctaactagggaacccactgc
ttaagcctcaataaagcttgccttgagtgcttcaagtagtgtgtgcccgtctgttgtgtgactctggtaactagagatccctcagacccttttagtcagtg
tggaaaatctctagcagtggcgcccgaacagggacttgaaagcgaaagggaaaccagaggagctctctcgacgcaggactcggcttgctgaag
cgcgcacggcaagaggcgaggggcggcgactggtgagtacgccaaaaattttgactagcggaggctagaaggagagagatgggtgcgagag
cgtcagtattaagcgggggagaattagatcgcgatgggaaaaaattcggttaaggccagggggaaagaaaaaatataaattaaaacatatagtatg
ggcaagcagggagctagaacgattcgcagttaatcctggcctgttagaaacatcagaaggctgtagacaaatactgggacagctacaaccatccc
ttcagacaggatcagaagaacttagatcattatataatacagtagcaaccctctattgtgtgcatcaaaggatagagataaaagacaccaaggaagc
tttagacaagatagaggaagagcaaaacaaaagtaagaccaccgcacagcaagcggccgctgatcttcagacctggaggaggagatatgaggg
acaattggagaagtgaattatataaatataaagtagtaaaaattgaaccattaggagtagcacccaccaaggcaaagagaagagtggtgcagaga
gaaaaaagagcagtgggaataggagctttgttccttgggttcttgggagcagcaggaagcactatgggcgcagcgtcaatgacgctgacggtaca
ggccagacaattattgtctggtatagtgcagcagcagaacaatttgctgagggctattgaggcgcaacagcatctgttgcaactcacagtctggggc
atcaagcagctccaggcaagaatcctggctgtggaaagatacctaaaggatcaacagctcctggggatttggggttgctctggaaaactcatttgca
ccactgctgtgccttggaatgctagttggagtaataaatctctggaacagatttggaatcacacgacctggatggagtgggacagagaaattaacaa
ttacacaagcttaatacactccttaattgaagaatcgcaaaaccagcaagaaaagaatgaacaagaattattggaattagataaatgggcaagtttgt
ggaattggtttaacataacaaattggctgtggtatataaaattattcataatgatagtaggaggcttggtaggtttaagaatagtttttgctgtactttc
tatagtgaatagagttaggcagggatattcaccattatcgtttcagacccacctcccaaccccgaggggacccgacaggcccgaaggaatagaagaag
aaggtggagagagagacagagacagatccattcgattagtgaacggatctcgacggtatcgctagcttttaaaagaaaaggggggattgggggg
tacagtgcaggggaaagaatagtagacataatagcaacagacatacaaactaaagaattacaaaaacaaattacaaaaattcaaaattttactagtg
attatcggatcaactttgtatagaaaagttggggttgcgccttttccaaggcagccctgggtttgcgcagggacgcggctgctctgggcgtggttccg
ggaaacgcagcggcgccgaccctgggtctcgcacattcttcacgtccgttcgcagcgtcacccggatcttcgccgctacccttgtgggccccccg
gcgacgcttcctgctccgcccctaagtcgggaaggttccttgcggttcgcggcgtgccggacgtgacaaacggaagccgcacgtctcactagtac
cctcgcagacggacagcgccagggagcaatggcagcgcgccgaccgcgatgggctgtggccaatagcggctgctcagcagggcgcgccga
gagcagcggccgggaaggggcggtgcgggaggcggggtgtggggcggtagtgtgggccctgttcctgcccgcgcggtgttccgcattctgca
agcctccggagcgcacgtcggcagtcggctccctcgttgaccgaatcaccgacctctctccccaggcaagtttgtacaaaaaagcaggctgccac
catgaaacgtccggcggccaccaagaaacatatgtgctttgccaagggtaccaatgttttaatggcggatgggtctattgaatgtattgaaaacattg
aggttggtaataaggtcatgggtaaagatggcagacctcgtgaggtaattaaattgcccagaggaagagaaactatgtacagcgtcgtgcagaaa
agtcagcacagagcccacaaaagtgactcaagtcgtgaagtgccagaattactcaagtttacgtgtaatgcgacccatgagttggttgttagaacac
ctcgtagtgtccgccgtttgtctcgtaccattaagggtgtcgaatattttgaagttattacttttgagatgggccaaaagaaagcccccgacggtagaat
tgttgagcttgtcaaggaagtttcaaagagctacccaatatctgaggggcctgagagagccaacgaattagtagaatcctatagaaaggcttcaaat
aaagcttattttgagtggactattgaggccagagatctttctctgttgggttcccatgttcgtaaagctacctaccagacttacgctccaattctttatt
ctagaactgactccaagtatttcacaaccaataaaaaaggagaaatatttgaactaaaagctgaactcaacaatgaaaagaaagaaaagagaaaggag
gctgtgaagaaagtgattgctgctatgaccgtggggaaggatgttagttctctctttccagacgtagtgaactgtatgcagactgacaatctggaacta
aagaagcttgtgtatctctacttgatgaactacgccaagagtcagccagacatggccatcatggctgtaaacagctttgtgaaggactgtgaagatcc
taatcctttgattcgagccttggcagtcagaaccatggggtgcatccgggtagacaaaattacagaatatctctgtgagccgctccgcaagtgcttga
aggatgaggatccctatgttcggaaaacagcagcagtctgcgtggcaaaactccatgatatcaatgcccaaatggtggaagatcagggatttctgg
attctctacgggatctcatagcagattcaaatccaatggtggtggctaatgccgtagcggcattatctgaaatcagtgagtctcacccaaacagcaac
ttacttgatctgaacccacagaacattaataagctgctgacagccctgaatgaatgcactgaatggggccagattttcatcctggactgcctgtctaatt
acaaccctaaagatgatcgggaggctcagagcatctgtgagcgggtaactccccggctatcccatgccaactcagcagtggtgctttcagcggta
aaagtcctaatgaagtttccagaattgttacctaaggattctgactactacaatatgctgctgaagaagttagcccctccacttgtcactttgctgtctgg
ggagccagaagtgcagtatgtcgccctgaggaacatcaacttaattgtccagaaaaggcctgaaatcttgaagcaggaaatcaaagtcttctttgtg
aagtacaatgatcccatctatgttaaactagagaagttggacatcatgattcgtttggcatctcaagccaacattgctcaggttctggcagaactgaaa
gaatatgctacagaggtggatgttgactttgttcgaaaagctgtgcgggccattggacggtgtgccatcaaggtggagcaatctgcagagcgctgt
gtaagcacattgcttgatctaatccagaccaaagtgaattatgtggtccaagaagcaattgttgtcatcagggacatcttccgcaaataccccaacaa
gtatgaaagtatcatcgccactctgtgtgagaacttagactcgctggatgagccagatgctcgagcagctatgatttggattgtgggagaatatgctg
aaagaattgacaatgcagatgagttactagaaagcttcctggagggttttcacgatgaaagcacccaggtgcagctcactctgcttactgccatagtg
aagctgtttctcaagaaaccatcagaaacacaggagctagtccagcaggtcttgagtttggcaacacaggattctgataatcctgaccttcgagacc
ggggctatatttattggcgccttctctcaactgaccctgttacagctaaagaagtagtcttgtctgagaagccactgatctctgaggagacggacctta
ttgagccaactctgctggatgagctaatctgccacattggttctttggcctctgtgtatcataagcctcccaatgcttttgtggaaggaagtcatggaatt
catcgtaaacacttgccaattcatcatgggagcactgatgcaggtgacagccctgttggcactaccactgcaacgaacctggaacagcctcaggtt
atcccctctcaaggtgatcttctaggggatcttttaaaccttgacctcggtcccccagtcaatgtgccacaggtgtcctccatgcagatgggagcagt
ggatctcctaggaggaggactagatagtctgcttggcagtgaccttggcgggggcattggaggaagtccggcagtgggacaatccttcatcccat
catcggtccctgcaacctttgctccttcacctacacctgctgtggtcagcagtggactgaatgacctgtttgaactctccacagggataggcatggca
cctggtggatatgtggctcctaaggctgtctggctacctgcagtaaaggctaaaggcttggagatttccggaacatttactcaccgccaagggcaca
tctatatggaaatgaacttcaccaataaagctctgcagcacatgacagattttgcaatccagtttaacaaaaatagctttggtgtcatccccagcactcc
tctggccatccatacaccactgatgccaaaccagagcattgatgtctccctgcctctcaataccttgggcccagtcatgaagatggaacctctgaata
acctccaggtggctgtgaaaaacaatatcgatgtcttctacttcagctgcctcatcccactcaatgtgctttttgtagaagatggcaaaatggagcgcc
aggtcttccttgcaacatggaaggatattcccaatgaaaatgaacttcagtttcagattaaggaatgtcatttaaatgctgacactgtttccagcaagttg
caaaacaacaatgtttatactattgccaagaggaatgtggaagggcaggacatgctgtaccaatccctgaagctcactaatggcatttggattttggc
cgaactacgtatccagccaggaaaccccaattacacgctgtcactgaagtgtagagctcctgaagtctctcaatacatctatcaggtctacgacagc
attttgaaaaacggaagcggagccacgaacttctctctgttaaagcaagcaggagatgttgaagaaaaccccgggcctatggtgagcaagggcg
aggaggataacatggccatcatcaaggagttcatgcgcttcaaggtgcacatggagggctccgtgaacggccacgagttcgagatcgagggcga
gggcgagggccgcccctacgagggcacccagaccgccaagctgaaggtgaccaagggtggccccctgcccttcgcctgggacatcctgtccc
ctcagttcatgtacggctccaaggcctacgtgaagcaccccgccgacatccccgactacttgaagctgtccttccccgagggcttcaagtgggagc
gcgtgatgaacttcgaggacggcggcgtggtgaccgtgacccaggactcctccctgcaggacggcgagttcatctacaaggtgaagctgcgcg
gcaccaacttcccctccgacggccccgtaatgcagaagaagaccatgggctgggaggcctcctccgagcggatgtaccccgaggacggcgcc
ctgaagggcgagatcaagcagaggctgaagctgaaggacggcggccactacgacgctgaggtcaagaccacctacaaggccaagaagcccg
tgcagctgcccggcgcctacaacgtcaacatcaagttggacatcacctcccacaacgaggactacaccatcgtggaacagtacgaacgcgccga
gggccgccactccaccggcggcatggacgagctgtacaagtaaacccagctttcttgtacaaagtggtgataatcgaattccgataatcaacctctggatt
acaaaatttgtgaaagattgactggtattcttaactatgttgctccttttacgctatgtggatacgctgctttaatgcctttgtatcatgctattgcttcc
cgtatggctttcattttctcctccttgtataaatcctggttgctgtctctttatgaggagttgtggcccgttgtcaggcaacgtggcgtggtgtgcactgt
gtttgctgacgcaacccccactggttggggcattgccaccacctgtcagctcctttccgggactttcgctttccccctccctattgccacggcggaactc
atcgccgcctgccttgcccgctgctggacaggggctcggctgttgggcactgacaattccgtggtgttgtcggggaagctgacgtcctttccatgg
ctgctcgcctgtgttgccacctggattctgcgcgggacgtccttctgctacgtcccttcggccctcaatccagcggaccttccttcccgcggcctgct
gccggctctgcggcctcttccgcgtcttcgccttcgccctcagacgagtcggatctccctttgggccgcctccccgcatcgggaattcccgcggttc
gaattctaccgggtaggggaggcgcttttcccaaggcagtctggagcatgcgctttagcagccccgctgggcacttggcgctacacaagtggcct
ctggcctcgcacacattccacatccaccggtaggcgccaaccggctccgttctttggtggccccttcgcgccaccttctactcctcccctagtcagg
aagttcccccccgccccgcagctcgcgtcgtgcaggacgtgacaaatggaagtagcacgtctcactagtctcgtgcagatggacagcaccgctg
agcaatggaagcgggtaggcctttggggcagcggccaatagcagctttgctccttcgctttctgggctcagaggctgggaaggggtgggtccgg
gggcgggctcaggggcgggctcaggggcggggcgggcgcccgaaggtcctccggaggcccggcattctgcacgcttcaaaagcgcacgtct
gccgcgctgttctcctcttcctcatctccgggcctttcgacctcacgtgcgcatgattgaacaagatggattgcacgcaggttctccggccgcttggg
tggagaggctattcggctatgactgggcacaacagacaatcggctgctctgatgccgccgtgttccggctgtcagcgcaggggcgcccggttcttt
ttgtcaagaccgacctgtccggtgccctgaatgaactgcaagacgaggcagcgcggctatcgtggctggccacgacgggcgttccttgcgcagc
tgtgctcgacgttgtcactgaagcgggaagggactggctgctattgggcgaagtgccggggcaggatctcctgtcatctcaccttgctcctgccga
gaaagtatccatcatggctgatgcaatgcggcggctgcatacgcttgatccggctacctgcccattcgaccaccaagcgaaacatcgcatcgagc
gagcacgtactcggatggaagccggtcttgtcgatcaggatgatctggacgaagagcatcaggggctcgcgccagccgaactgttcgccaggct
caaggcgagcatgcccgacggcgaggatctcgtcgtgacccatggcgatgcctgcttgccgaatatcatggtggaaaatggccgcttttctggatt
catcgactgtggccggctgggtgtggcggaccgctatcaggacatagcgttggctacccgtgatattgctgaagagcttggcggcgaatgggctg
accgcttcctcgtgctttacggtatcgccgctcccgattcgcagcgcatcgccttctatcgccttcttgacgagttcttctgagcgggactctgggtac
ctttaagaccaatgacttacaaggcagctgtagatcttagccactttttaaaagaaaaggggggactggaagggctaattcactcccaacgaagaca
agatctgctttttgcttgtactgggtctctctggttagaccagatctgagcctgggagctctctggctaactagggaacccactgcttaagcctcaataa
agcttgccttgagtgcttcaagtagtgtgtgcccgtctgttgtgtgactctggtaactagagatccctcagacccttttagtcagtgtggaaaatctctag
cagtagtagttcatgtcatcttattattcagtatttataacttgcaaagaaatgaatatcagagagtgagaggaacttgtttattgcagcttataatggtt
acaaataaagcaatagcatcacaaatttcacaaataaagcatttttttcactgcattctagttgtggtttgtccaaactcatcaatgtatcttatcatgtc
tggctctagctatcccgcccctaactccgcccatcccgcccctaactccgcccagttccgcccattctccgccccatggctgactaattttttttatttat
gcagaggccgaggccgcctcggcctctgagctattccagaagtagtgaggaggcttttttggaggcctagggacgtacccaattcgccctatagtgagtc
gtattacgcgcgctcactggccgtcgttttacaacgtcgtgactgggaaaaccctggcgttacccaacttaatcgccttgcagcacatccccctttcg
ccagctggcgtaatagcgaagaggcccgcaccgatcgcccttcccaacagttgcgcagcctgaatggcgaatgggacgcgccctgtagcggcg
cattaagcgcggcgggtgtggtggttacgcgcagcgtgaccgctacacttgccagcgccctagcgcccgctcctttcgctttcttcccttcctttctc
gccacgttcgccggctttccccgtcaagctctaaatcgggggctccctttagggttccgatttagtgctttacggcacctcgaccccaaaaaacttgat
tagggtgatggttcacgtagtgggccatcgccctgatagacggtttttcgccctttgacgttggagtccacgttctttaatagtggactcttgttccaaac
tggaacaacactcaaccctatctcggtctattcttttgatttataagggattttgccgatttcggcctattggttaaaaaatgagctgatttaacaaaaattt
aacgcgaattttaacaaaatattaacgcttacaatttaggtggcacttttcggggaaatgtgcgcggaacccctatttgtttatttttctaaatacattcaaa
tatgtatccgctcatgagacaataaccctgataaatgcttcaataatattgaaaaaggaagagtatgagtattcaacatttccgtgtcgcccttattccctt
ttttgcggcattttgccttcctgtttttgctcacccagaaacgctggtgaaagtaaaagatgctgaagatcagttgggtgcacgagtgggttacatcga
actggatctcaacagcggtaagatccttgagagttttcgccccgaagaacgttttccaatgatgagcacttttaaagttctgctatgtggcgcggtatta
tcccgtattgacgccgggcaagagcaactcggtcgccgcatacactattctcagaatgacttggttgagtactcaccagtcacagaaaagcatctta
cggatggcatgacagtaagagaattatgcagtgctgccataaccatgagtgataacactgcggccaacttacttctgacaacgatcggaggaccga
aggagctaaccgcttttttgcacaacatgggggatcatgtaactcgccttgatcgttgggaaccggagctgaatgaagccataccaaacgacgagc
gtgacaccacgatgcctgtagcaatggcaacaacgttgcgcaaactattaactggcgaactacttactctagcttcccggcaacaattaatagactg
gatggaggcggataaagttgcaggaccacttctgcgctcggcccttccggctggctggtttattgctgataaatctggagccggtgagcgtgggtct
cgcggtatcattgcagcactggggccagatggtaagccctcccgtatcgtagttatctacacgacggggagtcaggcaactatggatgaacgaaat
agacagatcgctgagataggtgcctcactgattaagcattggtaactgtcagaccaagtttactcatatatactttagattgatttaaaacttcatttttaat
ttaaaaggatctaggtgaagatcctttttgataatctcatgaccaaaatcccttaacgtgagttttcgttccactgagcgtcagaccccgtagaaaagat
caaaggatcttcttgagatcctttttttctgcgcgtaatctgctgcttgcaaacaaaaaaaccaccgctaccagcggtggtttgtttgccggatcaagag
ctaccaactctttttccgaaggtaactggcttcagcagagcgcagataccaaatactgttcttctagtgtagccgtagttaggccaccacttcaagaac
tctgtagcaccgcctacatacctcgctctgctaatcctgttaccagtggctgctgccagtggcgataagtcgtgtcttaccgggttggactcaagacg
atagttaccggataaggcgcagcggtcgggctgaacggggggttcgtgcacacagcccagcttggagcgaacgacctacaccgaactgagata
cctacagcgtgagctatgagaaagcgccacgcttcccgaagagagaaaggcggacaggtatccggtaagcggcagggtcggaacaggagag
cgcacgagggagcttccagggggaaacgcctggtatctttatagtcctgtcgggtttcgccacctctgacttgagcgtcgatttttgtgatgctcgtca
ggggggcggagcctatggaaaaacgccagcaacgcggcctttttacggttcctggccttttgctggccttttgctcacatgttctttcctgcgttatcc
cctgattctgtggataaccgtattaccgcctttgagtgagctgataccgctcgccgcagccgaacgaccgagcgcagcgagtcagtgagcgagga
agcggaagagcgcccaatacgcaaaccgcctctccccgcgcgttggccgattcattaatgcagctggcacgacaggtttcccgactggaaagcg
ggcagtgagcgcaacgcaattaatgtgagttagctcactcattaggcaccccaggctttacactttatgcttccggctcgtatgttgtgtggaattgtga
gcggataacaatttcacacaggaaacagctatgaccatgattacgccaagcgcgcaattaaccctcactaaagggaacaaaagctggagctgcaa
gctt

### 4C. Methods of Making N- and C-Fusion Proteins

The above-described N-fusion and C-fusion proteins can be prepared using molecular cloning as known in the art. For example, topoisomerase based cloning (i.e., TOPO® cloning) or restriction enzyme based molecular cloning can be used to prepare the above-described N-fusion and C-fusion proteins. In one example, Gibson assembly can be used to prepare the above-described N-fusion and C-fusion proteins. In another example, cloning procedure known in the art are described by Sambrook et al, Molecular Cloning: A Laboratory Manual, Cold Springs Laboratory, Cold Springs Harbor, N.Y (1989).

The prepared nucleic acids for the N-fusion and C-fusion proteins can be introduced into a lentiviral transfer plasmid. Bacteria such as E. coli can be transformed with the resulting lentiviral transfer plasmid to obtain larger quantities of said plasmid.

### 5. Methods of Use

The above-described biosensor proteins are provided in an expression vector. Additionally, the above-described biosensor proteins can be expressed in cells such as stem cells. In some embodiments, the stem cells are mammalian stem cells such as human, murine, bovine, equine, feline, or rabbit cells. In one embodiment, the stem cells are human cells. In one embodiment, the stem cell is a human induced pluripotent stem cell (hiPSC)-derived neural progenitor cell (NPC). In some embodiments, the stem cells are ReNcell® VM or ReNcell® CX. In one embodiment, the cell a non-human primate cell. The non-human primate may be, for example, a monkey, such as a cynomolgus monkey, chimpanzee, etc.

Moreover, the biosensor proteins can be expressed stably or transiently in stem cells using methods as known in the art. For example, in one embodiment, the biosensor proteins are transiently transfected into cells using a transfection reagent such as Lipofectamine, FuGENE®, or ProteoJuice Protein Transfection Reagent. In another embodiment, the biosensor proteins are transduced stably into stem cells using a lentiviral vector expressing a C-fusion protein to generate a mock sensor cell that expressed C-fusion alone and transduced with a lentiviral particle encoding an N-fusion protein. Co-transduced cells can be enriched using fluorescence-activated cell sorting (FACS) and expanded. Note that lentiviral vectors are derived from the human immunodeficiency virus and, like that virus, integrate into the host genome providing the potential for very long-term gene expression.

In some embodiments, the above-described biosensor proteins are used in a method of monitoring neuronal differentiation using real-time imaging. In one embodiment, the imaging is live-cell imaging. Live-cell imaging can be performed according to methods known in the art. For example, during live-cell imaging the cells are incubated at the appropriate conditions (e.g., at 37° C. in a humidified atmosphere with 5% CO₂) and imaged with a fluorescent confocal microscope. Additionally, the biosensor proteins are selective for neuronal differentiation and can distinguish this from glial differentiation.

This method of real-time monitoring of neuronal differentiation is advantageous over current technologies because it is non-invasive. Standard methods of monitoring neuronal differential in the field involve destructive steps such as fixing cells and lysing cells. Additionally, there are limitations to single-cell level analysis, providing a barrier to in situ monitoring. Single-cell RNA sequencing (scRNA-seq, which enables single-cell level analysis) includes considerable time and costs, with most methods requiring cell destruction, this results in a loss of key spatial information.

In one embodiment, the method of monitoring of neuronal differentiation is used for drug screening. Drugs can be screened using the above-described biosensor proteins to identify those that promote neuronal differentiation and can be used to treat neurological diseases.

To aid in understanding the detailed description of the compositions and methods according to the disclosure, a few express definitions are provided to facilitate an unambiguous disclosure of the various aspects of the disclosure. Unless otherwise defined, all technical and scientific terms used herein have the same meaning as commonly understood by one of ordinary skill in the art to which this disclosure belongs.

As used herein, “operatively linked” refers to a first molecule joined to a second molecule, wherein the molecules are so arranged that the first molecule affects the function of the second molecule. The two molecules may or may not be part of a single contiguous molecule and may or may not be adjacent. For example, a promoter is operatively linked to a transcribable polynucleotide molecule if the promoter modulates transcription of the transcribable polynucleotide molecule of interest in a cell. Additionally, two portions of a transcription regulatory element are operatively linked to one another if they are joined such that the transcription-activating functionality of one portion is not adversely affected by the presence of the other portion. Two transcription regulatory elements may be operatively linked to one another by way of a linker nucleic acid (e.g., an intervening non-coding nucleic acid) or may be operatively linked to one another with no intervening nucleotides present.

As used herein, the singular forms “a,” “an,” and “the” include plural references unless the context clearly dictates otherwise.

As used herein, the terms “including,” “comprising,” “containing,” or “having” and variations thereof are meant to encompass the items listed thereafter and equivalents thereof as well as additional subject matter unless otherwise noted.

As used herein, the phrases “in one embodiment,” “in various embodiments,” “in some embodiments,” and the like are used repeatedly. Such phrases do not necessarily refer to the same embodiment, but they may unless the context dictates otherwise.

As used herein, the terms “and/or” or “/” means any one of the items, any combination of the items, or all of the items with which this term is associated.

As used herein, the word “substantially” does not exclude “completely,” e.g., a composition that is “substantially free” from Y may be completely free from Y Where necessary, the word “substantially” may be omitted from the definition of the disclosure.

As used herein, the term “each,” when used in reference to a collection of items, is intended to identify an individual item in the collection, but does not necessarily refer to every item in the collection. Exceptions can occur if explicit disclosure or context clearly dictates otherwise.

As used herein, the term “approximately” or “about,” as applied to one or more values of interest, refers to a value that is similar to a stated reference value. In some embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless indicated otherwise herein, the term “about” is intended to include values, e.g., weight percents, proximate to the recited range that are equivalent in terms of the functionality of the individual ingredient, the composition, or the embodiment.

As disclosed herein, a number of ranges of values are provided. It is understood that each intervening value, to the tenth of the unit of the lower limit, unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither, or both limits are included in the smaller ranges is also encompassed within the invention, subject to any specifically excluded limit in the stated range. Where the stated range includes one or both of the limits, ranges excluding either or both of those included limits are also included in the invention.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All methods described herein are performed in any suitable order unless otherwise indicated herein or otherwise clearly contradicted by context. In regard to any of the methods provided, the steps of the method may occur simultaneously or sequentially. When the steps of the method occur sequentially, the steps may occur in any order, unless noted otherwise. In cases in which a method comprises a combination of steps, each and every combination or sub-combination of the steps is encompassed within the scope of the disclosure, unless otherwise noted herein.

Each publication, patent application, patent, and other reference cited herein is incorporated by reference in its entirety to the extent that it is not inconsistent with the present disclosure. Publications disclosed herein are provided solely for their disclosure prior to the filing date of the present invention. Nothing herein is to be construed as an admission that the present invention is not entitled to antedate such publication by virtue of prior invention. Further, the dates of publication provided may be different from the actual publication dates, which may need to be independently confirmed.

It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

### Example 1. Materials and Methods

This Examples provides the materials and methods used in Examples 2-5.

### 1A. Cell Culture and Differentiation

HeLa and HEK293T cells were routinely maintained in Dulbecco's modified Eagle medium (DMEM; Corning) supplemented with 10% fetal bovine serum (Gibco), 1% minimum essential medium-non-essential amino acids (Gibco), and 1% penicillin-streptomycin solution (Thermo Fisher Scientific) at 37° C. in a humidified atmosphere with 5% CO2. hiPSC-derived NPCs were maintained in a mixture of neural basal medium (Gibco) and DMEM/F12 (Gibco) supplemented with 0.5% N2 (Gibco), 0.5% B27 supplement (Invitrogen), and 20 ng mL-1 bFGF (PeproTech). All cells were maintained at 37° C. in a humidified incubator with 5% CO₂. To induce cell differentiation, hiPSC-derived NPCs were seeded on Matrigel (Corning)-precoated plates 24 hours prior to experimentation. After 1 day of culture to promote cell attachment and spread, fresh hiPSC-derived NPC media without bFGF (differentiation medium) was added to stop proliferation and induce differentiation. The medium was changed with fresh differentiation media every 2 days during the differentiation process. The human neural progenitor cell line ReN was obtained from Merck and maintained in DMEM/F12 (Gibco) supplemented with 1% GlutaMax (Gibco), 2% B27, 20 ng mL-1 heparin (Sigma-Aldrich), 0.5 mg mL-1 gentamicin (Gibco), 20 ng mL-1 bFGF, and 20 ng mL-1 EGF (Sigma-Aldrich). For ReN cell differentiation, a similar protocol was used with hiPSC-NSCs. ReN cells were seeded on Matrigel-precoated plates 24 h prior to experimentation. After 1 day of culture to promote cell attachment and spread, fresh ReN cell medium without bFGF (differentiation medium) was added to stop proliferation and induce differentiation. The medium was changed with fresh differentiation medium every 2 days during differentiation.

### 1B. Plasmids and Cloning

DNA cloning was performed according to the standard protocols using the gene-specific primers, listed in Table 4. Plasmids containing full-length human GRIA2 and AP2 genes were purchased from Korea human gene bank (hMU000638 and hMU008349). The coding sequences for Ic-NLS₁₀₋₁₈ in pJHJ015 plasmid ((Ryu, E. et al., Int. J. Mol. Sci. 2021, 22, 4747; E. Lee et al., Analyst 2020, 145, 5571; H. Jeon et al., Anal. Chem. 2018, 90, 9779; and E. Lee et al, Y Kwon, Biosensors 2023, 13, 383) were amplified using a forward primer containing AgeI and SpeI restriction sites and reverse primer containing ApaI restriction sites. Amplified products were digested using AgeI and ApaI and then cloned into the pBI-CMV1 vector. EGFP in pEGFP-N1 was amplified using a forward primer containing ApaI restriction site and a reverse primer containing an XbaI restriction site. Amplified EGFP was introduced into the pBI-CMV1 vector containing Ic-CFN-NLS₁₀₋₁₈ using ApaI and XbaI to yield pBI-CMV1 containing I_{C}-CFN-NLS10-18-EGFP. Coding sequences for GRIA2 were amplified using a forward primer containing AgeI restriction site and a reverse primer containing SpeI restriction site. Amplified GRIA2 was subcloned into pBI-CMV1 vector containing I_{C}-CFN-NLS₁₀₋₁₈-EGFP using AgeI and SpeI sites to yield C-fusion. Coding sequences for AP2 were amplified using forward primer containing Sail restriction site and reverse primer containing EagI restriction site. Amplified products were digested using Sail and EagI and cloned into the pimJHJ0 vector (Id.). Coding sequences for NLS₁₋₉-I_{N}-AP2 were amplified using a forward primer containing Mu restriction site and a reverse primer containing EagI restriction site. Amplified NLS₁₋₉-I_{N}-AP2 was subcloned into a pBI-CMV1 vector containing C-fusion using Mu and EagI to yield pB-CMV1, which contained both N-fusion and C-fusion. The cDNA encoding the mNLS was introduced into the multiple cloning site of pEGFP-N1 using restriction enzymes Sal and BamHI to create mNLS-EGFP. The lentiviral transfer plasmid (VB220126-1209pca and VB211216-1218daz) encoding N-fusion and C-fusion was individually constructed by VectorBuilder.

TABLE 4
Oligonucleotide primers used in gene cloning.

| | | SEQ |
|---|---|---|
| Primer | Primer sequence (5′→3′) | ID NO |
| AgeI SpeI I_{C}- | GAGTGTTCACCGGTACTAGTATG | 25 |
| NLS₁₀₋₁₈-F | GTTTT | |
| I_{C}-NLS₁₀₋₁₈ | TCACCATGGGCCCATCCAGTTTC | 26 |
| ApaI-R | | |
| ApaI EGFP-F | GGGCCCATGGTGAGCAAGGGC | 27 |
| EGFP XbaI-R | TCTAGATTACTTGTACAGCTCGT | 28 |
| | CC | |
| AgeI Kozak | GTGGATGCTACCGGTACCATGGA | 29 |
| GRIA2-F | AATG | |
| GRIA2 SpeI-R | CAAGGTCATACTAGTAATTTTAA | 30 |
| | CACTT | |
| SalI AP2-F | CACATTAAAGATGTCGACTCATG | 31 |
| | ACTGACTCCAAG | |
| AP2 EagI-R | GTACTGGACCACGGCCGTTAGTT | 32 |
| | TTTCAAAATGC | |
| MluI NLS₁₋₉- | GCGGCACGCGTATGAAACGTCCG | 33 |
| I_{N}-AP2-F | GC | |
| NLS₁₋₉-I_{N}- | GAGTGCGGCCGTTAGTTTTTCAA | 34 |
| AP2 EagI-R | AATGC | |
| SalI mNLS | TCGACATGAAACGTCCGGCGGCC | 35 |
| BamHI-F | ACCAAGAAAGCTAGCTGTTTCAA | |
| | CGCTAGCGGCCAGGCGAAAAAGA | |
| | AGAAACTG | |
| SalI mNLS | CAGTTTCTTCTTTTTCGCCTGGC | 36 |
| BamHI-R | CGCTAGCGTTGAAACAGCTAGCT | |
| | TTCTTGGTGGCCGCCGGACGTTT | |
| | CATGTCGA | |
| HindIII | GGCTCGAAGCTTATGGGCAAGCA | 37 |
| hippocalcin-F | G | |
| hippocalcin | GAACCTGGGATATCTCAGAACTG | 38 |
| EcoRV-R | GG | |

### 1C. Detection of Hippocalcin Using CBBs in HeLa and HEK293T Cells

HeLa or HEK293T cells were grown in a 12-well glass bottom plate (Thermo Fisher Scientific) and transiently transfected using plasmids expressing hippocalcin sensors. The expression of the hippocalcin sensor protein was allowed to proceed at 37° C. for 24 hours, and the sensor cells were sequentially transfected using a plasmid expressing HPCA. The sensor cells were washed with DPBS, and their nuclei were stained with Hoechst 33 342 (2 μm). Then, the fluorescence images were obtained to monitor the fluorescence signal translocation.

1D. Human Hippocalcin Protein Transfection into HEK293T Cells for Dose-Dependent and Time-Dependent Assays

HeLa or HEK293T cells were grown in 12-well glass bottom plate (Thermo Fisher Scientific) and transiently transfected using hippocalcin sensor-expressing plasmid. The expression of the hippocalcin sensor protein was allowed to proceed at 37° C. for 24 hours, and then the sensor cells were washed with DPBS, and their nuclei were stained with Hoechst 33 342 (2 μm). The sensor cells were treated with purified human hippocalcin protein (Abbexa) with ProteoJuice reagent (Sigma-Aldrich). Protein transfection procedures followed the manufacturer's recommendations. The transfection mixtures consisted of ProteoJuice reagent, different concentrations of purified human hippocalcin, and Opti-MEM media (Thermo Fisher Scientific). The transfection mixture was added directly to sensor cells in different wells, and they were allowed to grow for 2 hours. After 2 hours of incubation with the cells, the transfection mixture was supplemented with complete media and monitored using Zeiss LSM-780 confocal microscope using a 20× objective lens.

### 1E. Lentiviral Vector Production

Lentiviral vector production was performed as previously described (R. H. Kutner et al., Nat. Protoc. 2009, 4, 495). Briefly, HEK293T Lenti-X cells were added to a 6-well plate (≈1.5 ×105 cells per well). The next day, the following plasmid DNA transfection mix (3 μg of total DNA; 4:3:1 transfer/packaging/envelope plasmid ratio) was prepared in a sterile 1.5-mL tube: 1.5 μg transfer plasmid containing N-fusion or C-fusion, 1.125 μg psPAX2 packaging plasmid (#12 260, Addgene), and 375 ng pMD2.G envelope plasmid (#12 259, Addgene). The plasmid DNA transfection mix was transfected into HEK293T Lenti-X cells using Lipofectamine3000 (Invitrogen) according to the manufacturer's instructions. The six-well plate was placed at 37° C. in a humidified incubator with 5% CO₂. After 3 days of transfection, 18 mL of conditioned medium was collected from the HEK293T Lenti-X six-well plate into a sterile 50-mL tube. Subsequently, the following solutions were added: 7.5 mL of a 50% PEG 6000 (Sigma) solution, 3.2 mL of a 4 M NaCl (Sigma) stock solution, and 3.3 mL of DPBS (Thermo Scientific). A final volume of 30 mL was obtained. The final PEG 6000 concentration was 8.5% and the final NaCl concentration was ≈0.3 m. The bottles were stored at 4° C. for 1.5 hours. The contents were mixed every 20-30 min. The bottles were centrifuged at 7000×g for 10 min at 4° C. after which a white pellet formed. The supernatant was carefully decanted and 176 μL of 50 mm Tris-HCl (pH 7.4) was added. The pellets were resuspended by vigorously pipetting the liquid and dispensing it back. The vector suspension was transferred into microtubes in aliquots of 20 μL. The tubes were snap-frozen in crushed dry ice and stored at −80° C.

1F. Generation of NSC-Based CBBs that Stably Expressed Hippocalcin Sensor Protein

Approximately 3×10⁴ ReN cells in 0.5 mL of culture medium were added to each well in a 48-well plate. The 48-well plate was incubated at 37° C. in a humidified incubator with 5% CO₂ for 24 h. The culture medium was discarded, the plate was washed with 0.5 mL of phosphate-buffered saline (PBS), and the medium was replaced with 0.5 mL of lentivirus-containing medium containing 8 μg mL⁻¹ polybrene. The 48-well plate was placed back at 37° C. in a humidified incubator with 5% CO₂. Subsequently, the viral particles infected the cells and stably integrated their genetic material into the host cell genome.

### 1G. FACS

ReN cells stably transduced with a C-fusion-expressing lentiviral vector were analyzed and isolated via FACS using S3e Cell Sorter (Bio-Rad). The isolated clones were expanded in a Matrigel-coated 35-mm dish containing culture media. C-fusion-expressing ReN cells that were stably transduced with an N-fusion-expressing lentiviral vector were analyzed and isolated via FACS using MoFlo Astrios EQ (Beckman Coulter). The isolated clones were expanded in a Matrigel-coated 35-mm dish containing culture media.

### 1H. Imaging of Sensor Cells

Green fluorescence translocation in live NSC-based sensor cells and the expression of neural lineage-specific markers (TuJ1, GFAP, and Olig2) in fixed sensor cells were imaged using Andor Dragonfly 2000 confocal microscope with Nikon 40× and 100× objective lens. At least three sets of experiments were performed, and ˜30 cells were analyzed in each experiment. HeLa and HEK293T cells transiently expressing N- and C-fusion were imaged using Zeiss LSM-780 confocal microscope using 20× and 63× objective lens.

### 1I. Immunofluorescence Staining

To evaluate the ability of CBBs to monitor neuronal differentiation, the cells were fixed with 4% formaldehyde solution for 10 min at room temperature, followed by washing with PBS thrice. Subsequently, the cells were permeabilized using 0.1% Triton X-100 in PBS for 10 min, and then nonspecific binding was blocked with 5% bovine serum albumin (BSA; Proliant Biologicals) in PBS for 1 hour at room temperature. Primary mouse antibodies against TuJ1 (1:200 dilution, Biolegend) were used for the cells. Moreover, primary rabbit antibodies against GFAP (1:200 dilution, Invitrogen) and Olig2 (1:200 dilution, Abcam) were used to evaluate the ability of CBBs to distinguish between neuronal and glial cell differentiation. Based on the manufacturer's protocol, the fixed samples were incubated overnight at 4° C. in a solution of these antibodies in PBS containing 1% BSA and 0.3% Triton X-100. After washing thrice with PBS, the samples were incubated for 1 h at room temperature in a solution of anti-rabbit secondary antibodies labeled Alexa Fluor 647 (1:500, Invitrogen), anti-mouse secondary antibody labeled with Alexa Fluor 594 (1:500, Cell Signaling Technology), and Hoechst 33 342 (2 μm, Thermo Fisher Scientific) to stain nuclei. After washing thrice, all samples were imaged using a confocal microscope.

### 1J. Western Blot Analysis

Western blotting was performed to determine the level of CPS product, mNLS-EGFP, using an anti-GFP antibody from Invitrogen. Western blotting results were normalized using β-actin as a representative of the housekeeping protein. B-actin expression level was determined by using an anti-β-actin antibody (Cell Signaling Technology). The Western blots were developed with the SuperSignal™ West Pico PLUS Chemiluminescent Substrate (Thermo Fisher Scientific), according to the manufacturer's protocols, and imaged using iBright Imaging Systems (Thermo Fisher Scientific).

### 1K. Gene Expression Analysis

To evaluate the monitoring of early-stage neuronal differentiation via CBBs, RT-PCR was performed to evaluate hippocalcin production during differentiation. NSC-sensor cells were cultured in a 24-well plate (5×10⁵ cells per well) containing proliferation medium, and differentiation medium was added later into the 24-well plate. Total RNA was extracted from these sensor cells at 0, 1, 2, 3, 5, and 7 days after replacing the medium. Reverse transcription reaction was performed using 1 μg RNA via the cDNA Synthesis kit (Bioneer, AccuPower Cycle Script RT PreMix) following the manufacturer's instructions. RT-PCR assay was completed on the StepOnePlus™ Real-Time PCR system (Applied Biosystems) using the gene-specific primers, listed in Table 2. All measurements were run in triplicate. The gene expression level was reported relative to that of the endogenous control gene glyceraldehyde 3-phosphate dehydrogenase.

TABLE 5
Oligonucleotide primers used in RT-PCR.

| | Forward | Reverse |
|---|---|---|
| Gene | primer (5′→3′) | primer (5′→3′) |
| GAPDH | CCGCATCTTCTTTTG | GCCCAATACGACCAA |
| | CGTCG | ATCCGT |
| | (SEQ ID NO: 39) | (SEQ ID NO: 40) |
| SOX2 | CGAGTGGAAACTTTT | TCTTCATGAGCGTCT |
| | GTCGG | TGGTT |
| | (SEQ ID NO: 41) | (SEQ ID NO: 42) |
| TUBB3 | GGACCCTGTGAGTAG | CAGCTCCGACAGATC |
| | CCAGTA | CAGT |
| | (SEQ ID NO: 43) | (SEQ ID NO: 44) |
| NEUROD1 | GTCTCCTTCGTTCAG | AAAGTCCGAGGATTG |
| | ACGCTT | AGTTGC |
| | (SEQ ID NO: 45) | (SEQ ID NO: 46) |
| HPCA | AGATGGTTTCGTCCG | CTTGCCGTCGTTGTT |
| | TGATG | TGTG |
| | (SEQ ID NO: 47) | (SEQ ID NO: 48) |

### 1L. Time-Lapse Microscopy

Time-lapse microscopy was performed as follows: NSC-sensor cells were cultured as monolayers on a glass-bottom 24-well plate (ibidi GmbH). The next day, culture medium was replaced with differentiation medium. After 24 hours, images were obtained every 30 minutes for the indicated time courses using Nikon T2500 inverted fluorescence microscope with 20× objective lens and an environmental chamber to maintain temperature (37° C.) and CO₂ concentration (5%).

### 1M. Statistical Analysis

All experiments were performed in triplicate and data were presented as the mean standard deviation or mean±standard error of mean. Line-scan profiles of fluorescence intensity in cells were analyzed using ImageJ. The PlotProfile feature of ImageJ was used to record the pixel intensities along the selected line, and the results were saved in a text format. The data for each profile were then imported into GraphPad Prism 8.0 (GraphPad Software). Prism 8.0 was used to plot generated data and perform statistical analysis. A comparison between different groups was performed using one-way analysis of variance followed by Tukey's multiple-comparison test. Values of p<0.05 were considered significant.

### Example 2. Design and Generation of a CBB Based on Intein-Mediated CPS for Monitoring Neuronal Differentiation

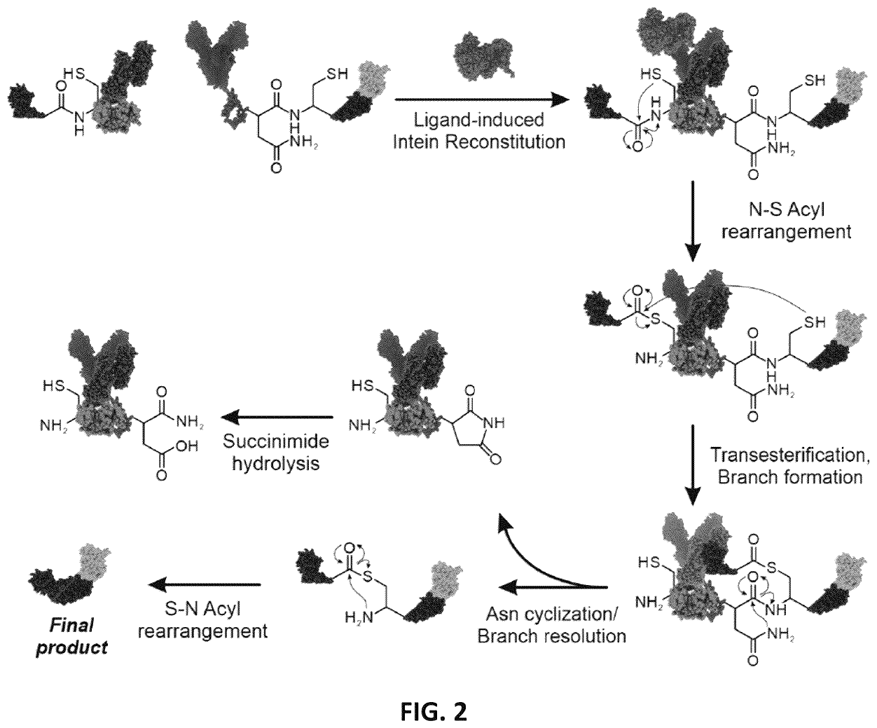

In this study, hippocalcin was used as the target of a CBB to monitor neuronal differentiation. To track neuronal differentiation, a reporting method was used wherein a fluorescence signal was translocated from the cytoplasm to the nucleus through intein-mediated reactions. A split-intein, vacuolar ATPase of Saccharomyces cerevisiae (Sce VMA) inteins was used to induce CPS, where PS is induced only when a target is present. The Sce VMA inteins are suitable inteins for inducing CPS owing to their low affinity for spontaneous PS and relatively fast reaction rate (reaction t_{1/2} of ≈10 min) (J. Zettler et al., FEBS Lett. 2009, 583, 909; J. Yang et al., Proc. Natl. Acad. Sci. U.S.A 2003, 100, 3513; N. H. Shah et al., Chem. Sci. 2014, 5, 446; N. Palanisamy et al., FEBS Lett. 2006, 580, 1853; D. Guan, M. Ramirez, Z. Chen, Biotechnol. Bioeng. 2013, 110, 2471; S. Brenzel et al., Biochemistry 2006, 45, 1571 and A. S. Aranko et al., Nat. Chem. Biol. 2013, 9, 616). (FIG. 2). The AP2 and GRIA2 were introduced separately into the C-terminus of N-intein (I_{N}) and N-terminus of C-intein (I_{C}) respectively, allowing for the heterodimerization of split-inteins by hippocalcin. AP2 directly binds to hippocalcin, and the AP2-hippocalcin complex combines with GRIA2 in hippocalcin-Ca²⁺ signaling (S. Y Park, et al., Stem Cell Rep. 2017, 8, 95; D. A. Brown et al., Neuron 2007, 53, 467; N. Helassa et al., Hum. Mol. Genet. 2017, 26, 2426; and C. L. Palmer et al., Neuron 2005, 47, 487). This interaction is induced by the myristoylation of hippocalcin, which is essential for the expression of nerve growth factors during neuronal differentiation of NSCs and is well-known to contribute to neuronal activities, including synaptic plasticity, learning, and memory (J. Zettler et al., FEBS Lett. 2009, 583, 909; J. Yang, G. C. Fox Jr et al., Proc. Natl. Acad. Sci. U.S.A 2003, 100, 3513; N. H. Shah et al., Chem. Sci. 2014, 5, 446; N. Palanisamy et al, Nat. Commun. 2019, 10, 4967; H. Iwai, et al, FEBS Lett. 2006, 580, 1853; D. Guan et al, Biotechnol. Bioeng. 2013, 110, 2471; S. Brenzel et al., Biochemistry 2006, 45, 1571; A. S. Aranko et al., Nat. Chem. Biol. 2013, 9, 616; and J. Jo et al., Nat. Neurosci. 2010, 13, 1216).

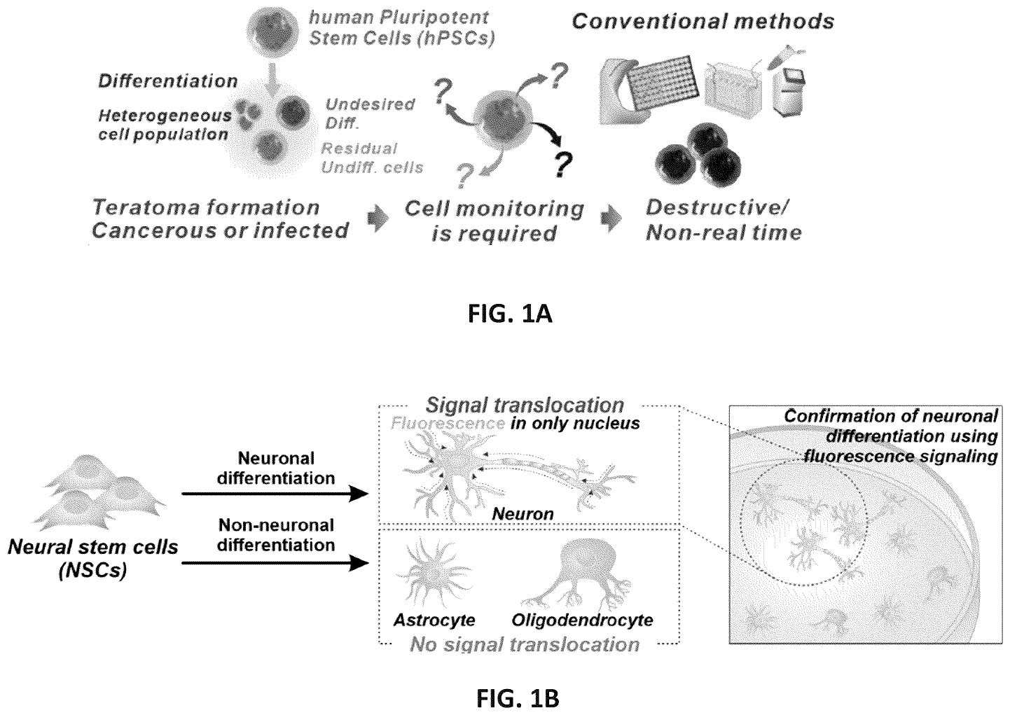

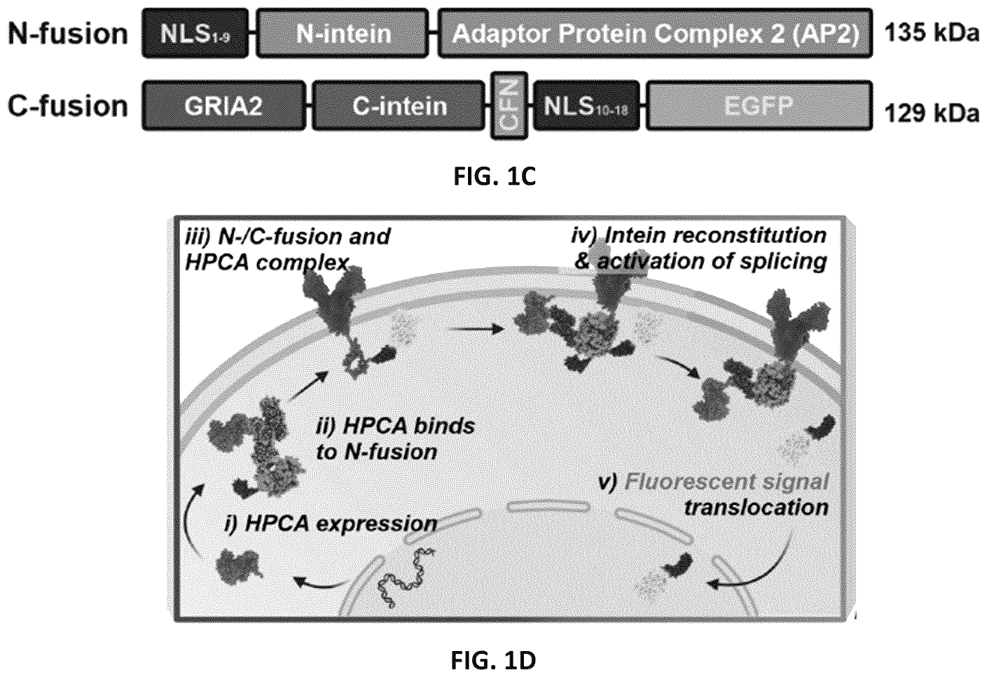

To detect the overexpression of hippocalcin via fluorescence signal translocation, the NLS (KRPAATKKAGQAKKKKLD; SEQ ID NO: 5) derived from the nucleoplasmin of Xenopus laevis (J. P. S. Makkerh et al., Curr. Biol. 1996, 6, 1025) was split at position 9 to generate NLS₁₋₉ and NLS₁₀₋₁₈. NLS₁₋₉ and NLS₁₀₋₁₈ were introduced separately as N and C-extein, respectively, and the optical reporter-enhanced green fluorescent protein (EGFP) was inserted at the C-terminus of NLS₁₀₋₁₈. The canonical “CFN” tripeptide sequence, which is the first three amino acids of the native C-extein sequence required for efficient PS, was present in the final product after the CPS reaction (S. W. Lockless et al., Proc. Natl. Acad. Sci. U.S.A 2009, 106, 10999 and A. J. Stevens et al., Proc. Natl. Acad. Sci. U.S.A 2017, 114, 8538). This means that the spliced NLS peptides were modified. To determine whether modified NLS (mNLS) peptides function like native NLS, the mNLS was evaluated using DeepLoc 2.0, a tool for subcellular localization prediction. The important sequence in mNLS required for nuclear localization was located in the C-terminus; therefore, the “CFN” tripeptide is not expected to interfere with the native function of the NLS. The probability of the entire sequence of mNLS located in the nucleus was considerably higher than that in other compartments; this indicates that the mNLS would be in the nucleus. HeLa cells were transfected with the gene encoding mNLS-EGFP to confirm the localization of the final product, and mNLS-EGFP was observed mainly in the nucleus. The line intensity histogram confirmed the localization of mNLS-EGFP in the nucleus, indicating that mNLS sufficiently induces the nuclear translocation of EGFP. Thus, hippocalcin sensor proteins, N-fusion and C-fusion (NLS₁-9-I_{N}-AP2 and GRIA2-I_{C}-CFN-NLS₁₀₋₁₈-EGFP, respectively), were prepared to generate CBBs for monitoring neuronal differentiation (FIG. 1B).

### Example 3. Characterization of CBBs for Hippocalcin Detection in Established Cell Lines

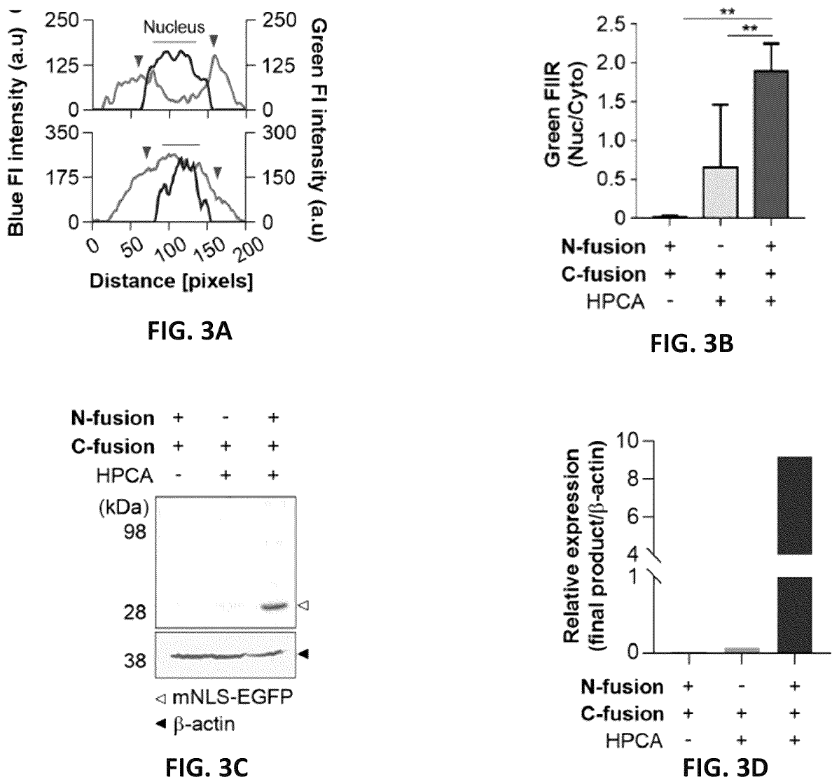

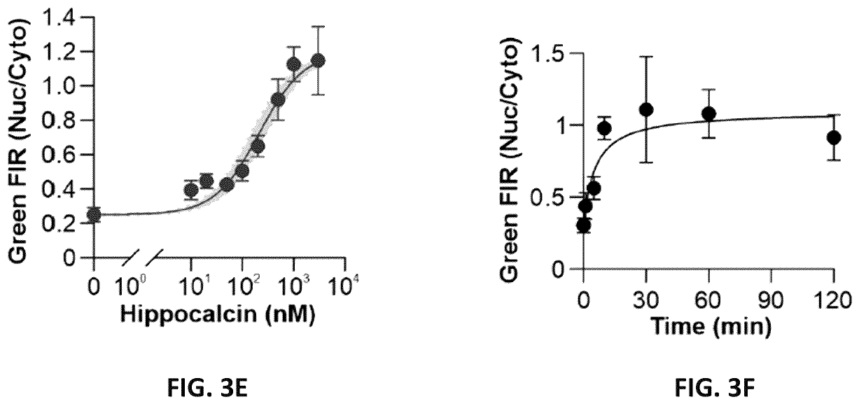

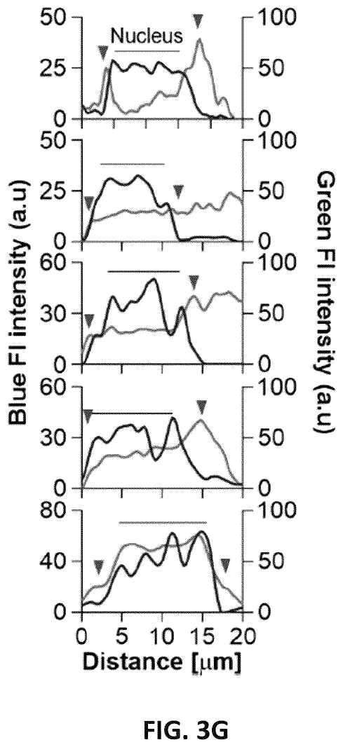

Prior to generating an NSC-based biosensor to monitor neuronal differentiation, N- and C-fusion-expressing HeLa and HEK293T cells were prepared to test their ability to detect hippocalcin. These cell lines were used to generate a test version of CBB since they have high transfection efficiency and are easy to grow and maintain (A. H. Hyman et al., Nature 2011, 480, 34 and E. V. Tan et al., Front. Bioeng. Biotechnol. 2021, 9, 796991). Both cell lines that express the hippocalcin sensor proteins were sequentially transfected with the hippocalcin-encoding plasmid because they do not express endogenous hippocalcin. A green fluorescent signal was obtained from the sensor construct and a blue fluorescent signal was obtained from Hoechst that stained the nuclei. The green fluorescence signal was located in the cytoplasm of sensor cells in the absence of hippocalcin and translocated to the nucleus when CBBs started expressing hippocalcin. Notably, the presence of hippocalcin did not induce the translocation of the green fluorescence signal in mock sensor cells that only expressed the C-fusion. This finding suggests that the C-fusion alone cannot mediate hippocalcin-triggered fluorescence signal translocation and that the additional N-fusion containing a hippocalcin-binding domain is essential for this process. These results showed that the observed translocation of the green fluorescence signal in sensor cells could be attributed to the intein-mediated CPS reaction induced by hippocalcin. The intracellular distribution of green fluorescence signal between the cytoplasm and nucleus was analyzed by comparing the fluorescence intensity profile before and after hippocalcin expression, which showed that the fluorescent cargo moved to the nucleus upon hippocalcin expression (FIG. 3A). Hippocalcin expression was associated with a 20-fold increase in the nucleus-to-cytoplasm (Nuc/Cyto) ratio of green fluorescence intensity of the sensor cells by inducing an intein-mediated CPS reaction. Mock sensor cells without N-fusion did not show nuclear translocation of the green fluorescence signal, as expected (FIG. 3B). Data was analyzed using one-way ANOVA using the Tukey multiple comparison test (** p<0.01). In addition, western blot analysis was performed to verify whether the translocation of the green fluorescence signal is mediated by the final product formed by the intein-mediated CPS (FIGS. 3C and 3D). β-actin was used as a loading control (FIG. 3C). The CPS product was detected only in sensor cells transfected with the hippocalcin-expressing plasmid.

Assessment of sensor cell performance involved an evaluation of detection limits and response times (FIGS. 3E-3G). HEK293 T cells expressing the hippocalcin sensor protein were treated with various concentrations of hippocalcin ranging from 0 to 3 μm using ProteoJuice transfection reagent to determine the ability of the CBB to detect hippocalcin during neuronal differentiation. This range of hippocalcin concentration was used because mature neurons typically express ≈3 μm of hippocalcin (V. Cherkas et al., PLoS One 2018, 13, e0194031 and M. Kobayashi, K. Takamatsu, Encyclopedia of Signaling Molecules, Springer, New York, NY, USA 2012). A noticeable response to the increasing hippocalcin concentration was observed based on the increase in green fluorescence signal migrating to the nucleus (FIG. 3E). The limit of detection (LOD) was determined to be 42 nm, indicating the desirable sensitivity of the CBB in detecting low concentrations of hippocalcin during neuronal differentiation. To evaluate the response time of the CBB, HEK293T cells expressing the hippocalcin sensor protein were treated with 1 μm hippocalcin. This concentration was selected because the green fluorescence intensity ratio (FIR) of Nuc/Cyto reached a plateau at this level. The translocation of the green fluorescence signal could be detected as early as 10 min after stimulation, and the green FIR of Nuc/Cyto reached a plateau ≈in 30 min (FIG. 3F). The intracellular distribution of green fluorescence signal between the cytoplasm and nucleus was analyzed using the fluorescence intensity profile following hippocalcin stimulation. The analysis showed that the migration of the fluorescent cargo to the nucleus began within 10 min and that the complete transfer of the fluorescence signal was achieved within 30 min (FIG. 3G).

Collectively, the result of this experiment unveils a new avenue for hippocalcin detection through the development of CBBs. The elegant integration of CPS-mediated reconstitution and instantaneous signal peptide activation enables a sensitive and rapid fluorescence readout of hippocalcin levels. Although this initial demonstration focuses on hippocalcin detection alone, the broader implications lie in applying these CBBs to monitor the dynamic process of neuronal differentiation. By visualizing and quantifying real-time changes in hippocalcin expression within differentiating NSCs, insight can be gained into the spatiotemporal regulation of neurogenesis. This holds immense potential to advance the current understanding of neuronal development and unlock new therapeutic avenues in research on regenerative medicine and neurological diseases.

### Example 4. Monitoring Neuronal Differentiation Using CBBs Based on hiPSC-Derived NPCs

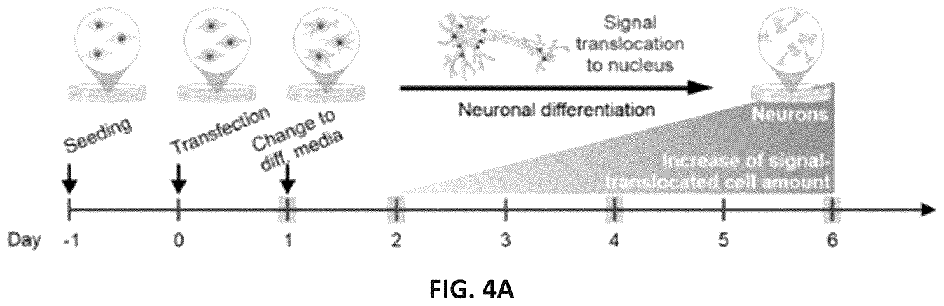

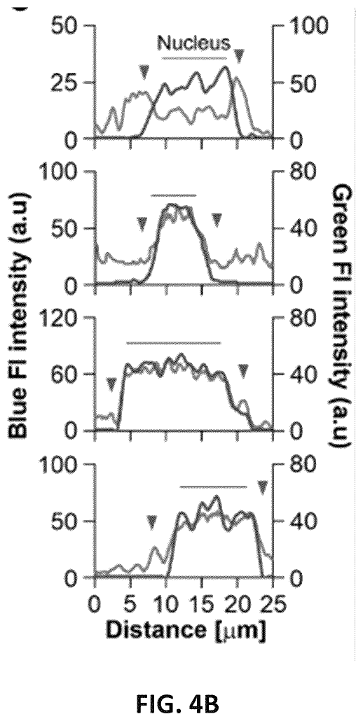

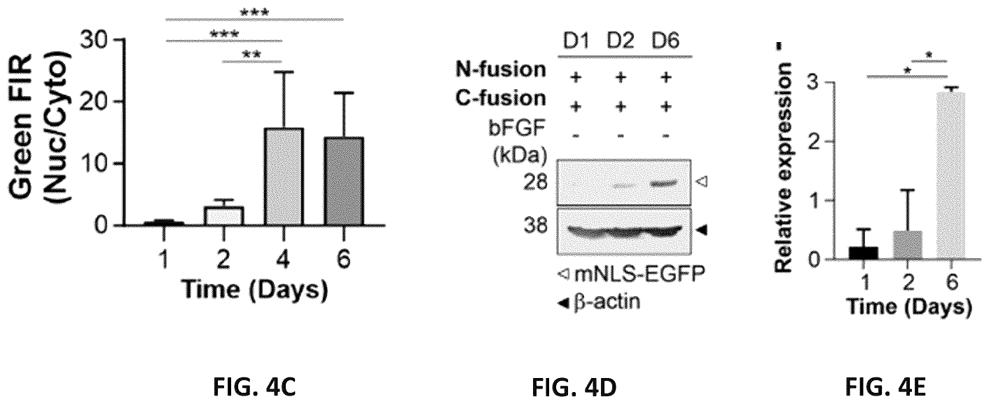

To monitor neuronal differentiation, CBBs expressing the hippocalcin sensor proteins (N- and C-fusion) were prepared using human induced pluripotent stem cell (hiPSC)-derived neural progenitor cells (NPCs). iPSC-NPCs were transiently transfected with the hippocalcin sensor plasmid to produce CBBs at day −1. After 1 day, fresh medium without FGF basic (differentiation medium) was added to induce neuronal differentiation. The medium was replaced with fresh differentiation medium every 2 days during differentiation. CBBs using iPSC-derived NPCs were cultured in basic fibroblast growth factor (bFGF)-free differentiation medium for 6 days to monitor neuronal differentiation via hippocalcin-triggered nuclear translocation of fluorescence signal (FIG. 4A). Subsequently, sensor cells were fixed and subjected to class III β-tubulin (TuJ1) staining, a representative marker for neuronal differentiation, at each time point. This enables a comparison of changes in the subcellular location of the green fluorescence signal with TuJ1 expression levels. Notably, green fluorescence signal translocation to the nucleus was detected as early as day 2 of differentiation, and TuJ1 expression was seen starting from day 4 of differentiation of hiPSC-derived NPCs expressing the hippocalcin sensor protein. The fluorescence intensity profiles were analyzed across the selected cells marked by a white line, revealing an overlap between the green fluorescence and blue fluorescence signals from day 2 onward (FIG. 4B). Note that the data were analyzed using one-way ANOVA followed by Tukey's multiple comparison tests (**p<0.01, ***p<0.001). This result signifies that nuclear translocation of fluorescent cargo occurred before TuJ1 expression. As the cells underwent differentiation, the green FIR of Nuc/Cyto in the sensor cells gradually increased (FIG. 4C). Again, the data were analyzed using one-way ANOVA followed by Tukey's multiple comparison tests (**p<0.01, ***p<0.001). Western blotting was performed to verify that the green fluorescence signal translocation is mediated by the CPS reaction facilitated by split-intein, wherein the splicing product mNLS-EGFP was detected from day 2 onward (FIGS. 4C and 4D). Consequently, these results suggest that green fluorescence translocation was induced by hippocalcin-triggered CPS reaction. These results also suggest that CBBs using hiPSC-derived NPCs can be used to monitor early-stage neurodevelopment during neuronal differentiation.

### Example 5. Establishment of NSC-Based CBBs Stably Expressing the Hippocalcin Sensor for Evaluation of Neural Lineage Differentiation in Real-Time

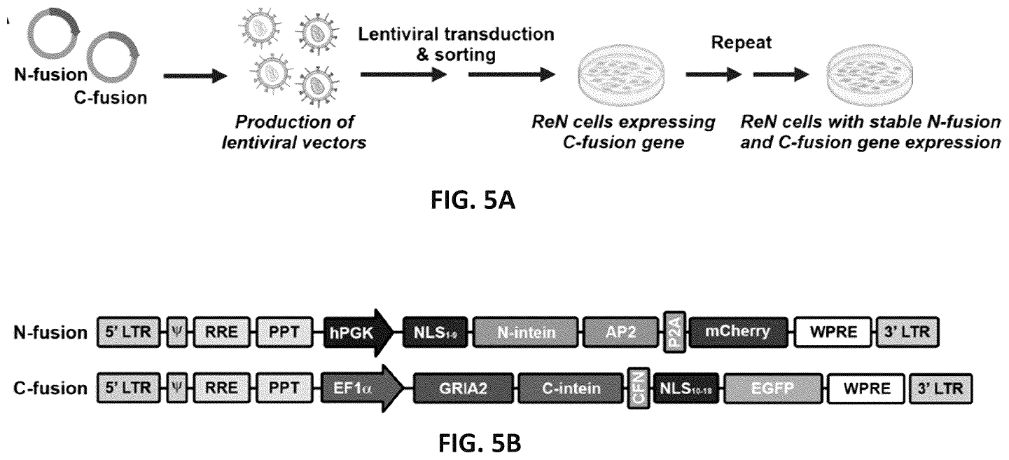

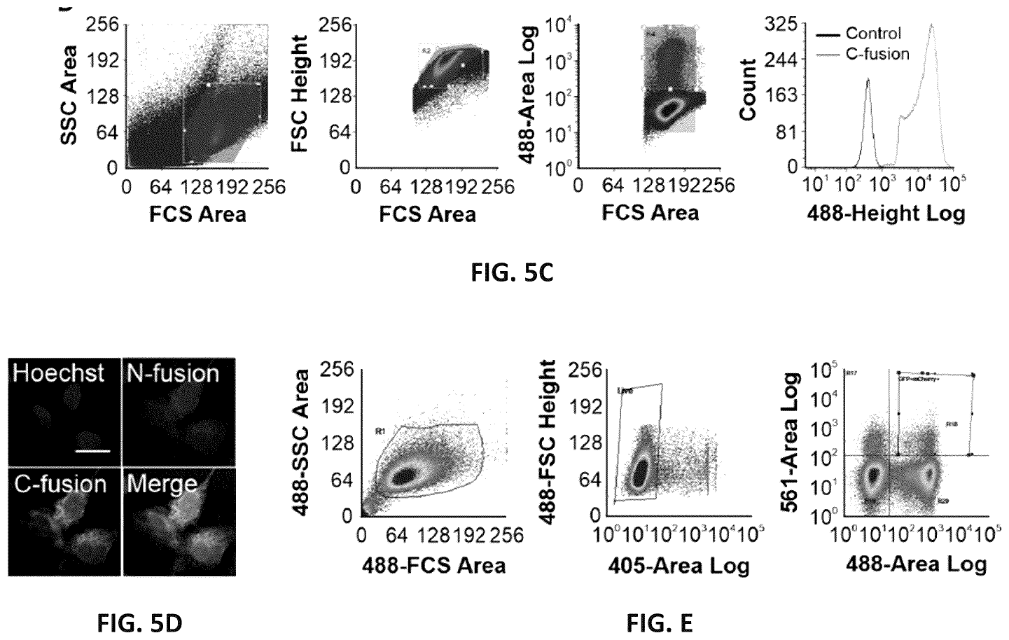

Biosensors developed from NPCs derived from hiPSCs, which expressed the hippocalcin sensor proteins (N- and C-fusions), were successfully used to monitor cell differentiation into neurons. This process was observed by tracking the movement of a green fluorescence signal. These results indicate that it is potentially feasible to monitor neural differentiation in neurological disease models using patient-derived iPSCs, providing important information for understanding the progression of neurological diseases and developing new therapeutic strategies. Furthermore, ReN cells were used to build a viable sensor cell line carrying hippocalcin sensor proteins to assess the quantitative analysis capabilities of the CBB (FIGS. 5A-5E). ReN cells are a well-characterized neural stem cell line and provide a consistent and homogeneous population of neural cells, which can be advantageous for assessing the ability of the CBB to determine neuronal and glial differentiation. To generate a stable cell line expressing the hippocalcin sensor protein, lentiviral particles, including N- and C-fusion, were individually generated using Lenti-X 293T cells that were transiently co-transfected with transfer (N-fusion and C-fusion vector, respectively), envelope, and packaging plasmids. The NSCs were first infected and transduced stably using a lentiviral vector expressing C-fusion to generate a mock sensor cell that expressed C fusion alone (FIG. 5C). For the FACS analysis of these cells, the first gate was set on FSC/SSC to include the NSC population. The second gate was on FSC-H vs FSC-A based on the NSC gate doublet exclusion. Finally, C-fusion-presenting cells were sorted from the single cells.

Next, C-fusion-expressing NSCs were transduced using a lentiviral particle encoding N-fusion with mCherry. mCherry was introduced to isolate stable cell lines using FACS and was specifically designed to cleave at N-fusion after its expression, ensuring that there is no interference with N-fusion (FIG. 5B). Co-transduced cells were enriched using FACS and then expanded; the red and green fluorescence signals from N- and C-fusion were confirmed via fluorescence microscopy, respectively (FIGS. 5D and 5E). Note that out of the C-fusion subpopulation, the mCherry-expressing NSCs were sorted (FIG. 5E).

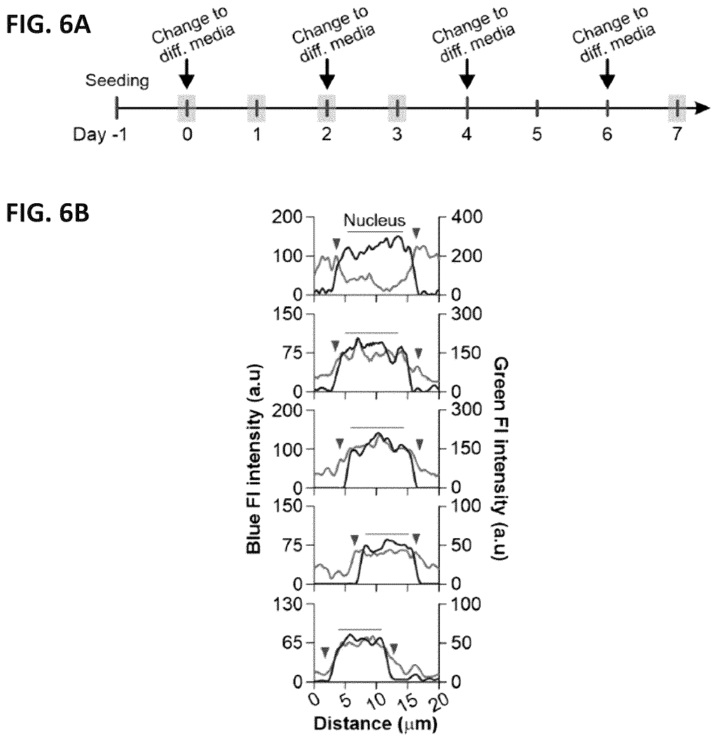

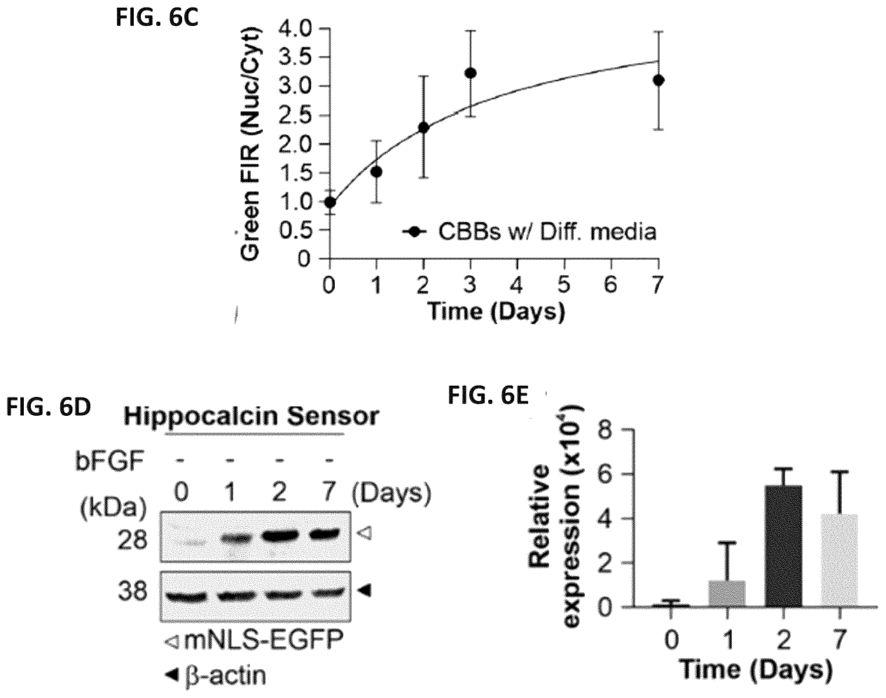

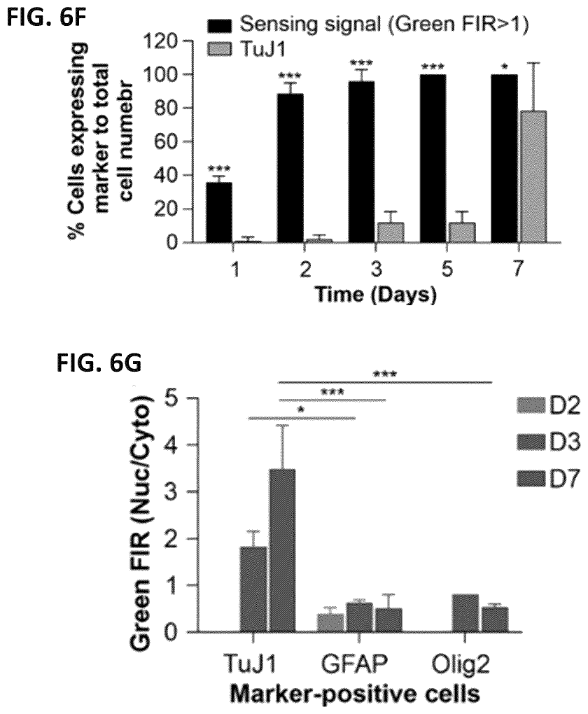

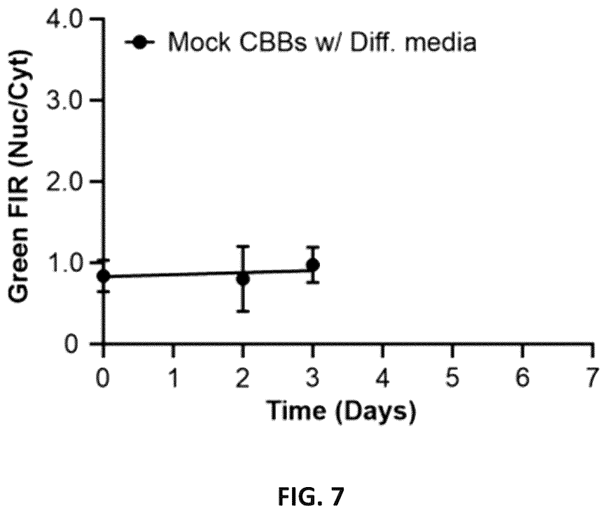

After generating CBBs that stably expressed the hippocalcin sensor proteins using NSCs, these NSC-based CBBs were used for non-invasive monitoring of neuronal differentiation. The translocation of the green fluorescence signal in NSC-based CBBs was assessed on days 0, 1, 2, 3, and 7 of differentiation in live cells (FIG. 6A). In this study, after 1 day, fresh medium without bFGF (basic fibroblast growth factor) (differentiation medium) was added to induce neuronal differentiation. The medium was replaced with fresh differentiation medium every 2 days during differentiation. At day 0, green fluorescence was distributed throughout the cytoplasm; after differentiation, the green fluorescence signal gradually moved to the nucleus. To qualitatively evaluate green fluorescence translocation, the line scanning profiles of fluorescence intensity of the selected cells were analyzed (FIG. 6B). The fluorescence intensity profiles across the selected cells show high contrast between the nucleus and cytoplasm regions at day 0. From day 1 of differentiation, the green fluorescence intensity began to overlap with the blue fluorescence intensity, indicating that the green fluorescence signal had migrated to the nucleus. As a result of green FIR analysis of Nuc/Cyto, it was confirmed to be 0.8 on day 0 of differentiation and showed a clear increasing trend from 0.8 to 3.2 by day 3, and there was no significant difference until day 7 (FIG. 6C). ReN cells are known to stop growing within 2 to 3 days after replacement with differentiation medium, and differentiation of ReN cells into specific cells can be distinguished after 4 days (R. Hoffrogge et al., Proteomics 2006, 6, 1833). This means that the green FIR of Nuc/Cyto eventually became saturated on day 3 of differentiation, rendering differentiation monitoring possible before visualization of morphological change into neurons and glial cells. To evaluate whether the split-intein-mediated CPS reaction induced green fluorescence translocation, the mock sensor that expressed C-fusion alone was monitored using confocal microscopy during differentiation; sufficient nuclear translocation of green fluorescence was not detected even in the presence of hippocalcin (FIG. 7). These results strongly suggest that green fluorescence translocation was induced by hippocalcin-triggered intein-mediated CPS reaction. Western blotting was performed to further validate the production of the sensor product. Expression of the sensor product was detected from day 2 of differentiation (FIGS. 6D and 6E; data were analyzed using two-way ANOVA followed by Sidak's multiple comparison tests (*p<0.05, ***p<0.001)). This suggests that green fluorescence translocation was induced by hippocalcin-triggered CPS reaction. Additionally, the formation of mNLS-EGFP showed significant increases at all the time points compared with day 0 (*p<0.05, one-way ANOVA) (FIG. 6E). Taken together, this sensing system can non-invasively monitor neuronal differentiation in NSCs via sensitive detection of hippocalcin during differentiation. The signal was detected from day 2 of differentiation, a relatively early stage of neuronal differentiation. These results are consistent with the sensing result using hiPSC-derived NPC-based CBBs that transiently express the hippocalcin sensor.

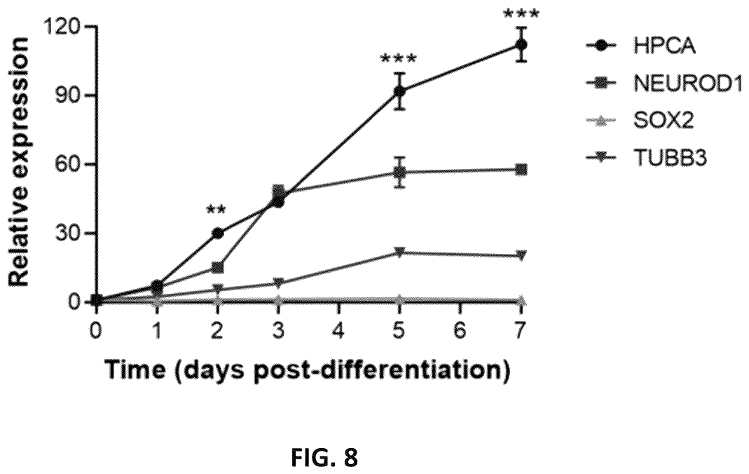

Next, the sensor cells were stained using TuJ1 and compared the signal with the expression of TuJ1 to validate whether the sensing product was generated before TuJ1 expression (FIG. 6F; note cells that stained positive for TuJ1 was calculated at each time point, respectively). The number of cells with a green FIR of Nuc/Cyto >1 was calculated at each time point, considering that the background signal of CBBs was ˜0.8 at day 0 of differentiation (FIG. 6C). The number of sensor cells that stained positive for TuJ1 was also calculated. In total, 40% of sensor cells began to show green fluorescence translocation on day 1 of differentiation. Most of the sensor cells (88%) exhibited nuclear translocation of green fluorescence on day 2 of differentiation, and the signal consistently increased until day 7 of differentiation. In contrast, TuJ1 expression was found in only 2% of sensor cells on day 2 of differentiation and increased to 12% on day 3 of differentiation. As expected, the translocation of the green fluorescence preceded TuJ1 expression. Additionally, RT-PCR analysis showed that hippocalcin (HPCA) expression was significantly increased in the early stages of differentiation, especially before neurogenic differentiation-1 (NEUROD1) and TUBB3 expression (FIG. 8; mRNA levels were normalized to GAPDH. Data are means±SEM (n=3); changes are relative to controls. **, P <0.01; ***, P<0.001 (two-way ANOVA, Tukey's multiple comparisons test). Each sample mean was compared with the control (HPCA) mean at that time point in time). Besides promoting neuronal differentiation in NSCs, NeuroD1 also plays a pivotal role in driving neuronal maturation and fostering the survival of neurons (C. Boutin et al., Proc. Natl. Acad. Sci. U.S.A 2010, 107, 1201). This finding revealed that the sensor platform could detect hippocalcin expression and monitor neuronal differentiation in the early stage before NeuroD1.

To investigate how effectively NSC-based CBBs can differentiate neural lineages, IF staining was performed to detect the expression of lineage-specific markers. Subsequently, the green FIR of Nuc/Cyto was assessed in sensor cells that were stained positive for TuJ1, glial fibrillary acidic protein (GFAP), and oligodendrocyte transcription factor (Olig2), respectively to evaluate the discriminative ability of CBBs in relation to distinguishing neural lineage differentiation (FIG. 6G). The data were analyzed using two-way ANOVA using the Tukey multiple comparison test (*p<0.05, ***p<0.001). GFAP expression in sensor cells was observed from day 2 of differentiation, and sensor cells that stained positive for GFAP did not show green fluorescence translocation. Olig2 expression in sensor cells was observed from day 3 of differentiation, and no green fluorescence translocation was detected in sensor cells that stained positive for Olig2. The number of cells with a green FIR of Nuc/Cyto >1 in sensor cells that individually stained positive for the neural lineage-specific marker was counted. All sensor cells that stained positive for TuJ1 showed green fluorescence translocation, whereas those that stained positive for GFAP and Olig2 did not show green fluorescence translocation. These results indicate thatNSC-based CBBs can not only monitor neuronal differentiation in the early stage, but also distinguish between neuronal and glial differentiation.

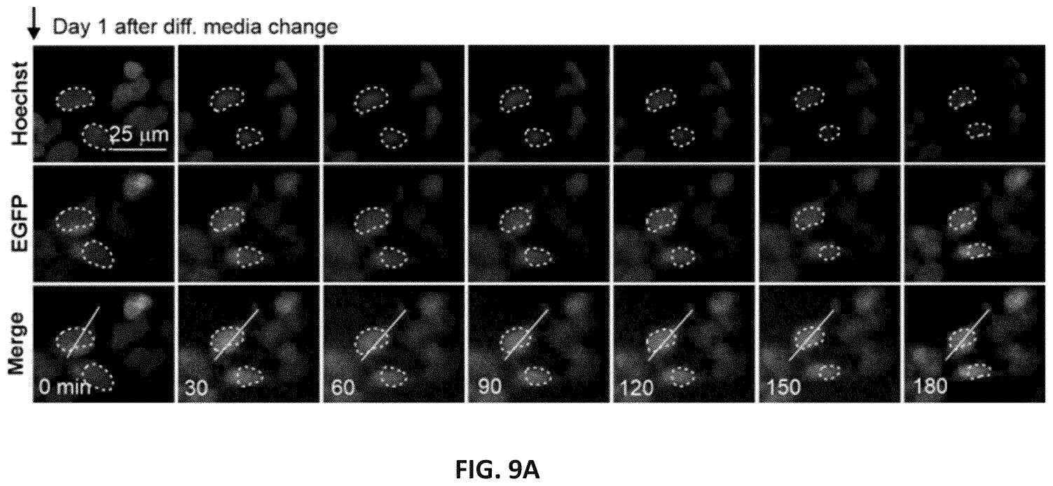

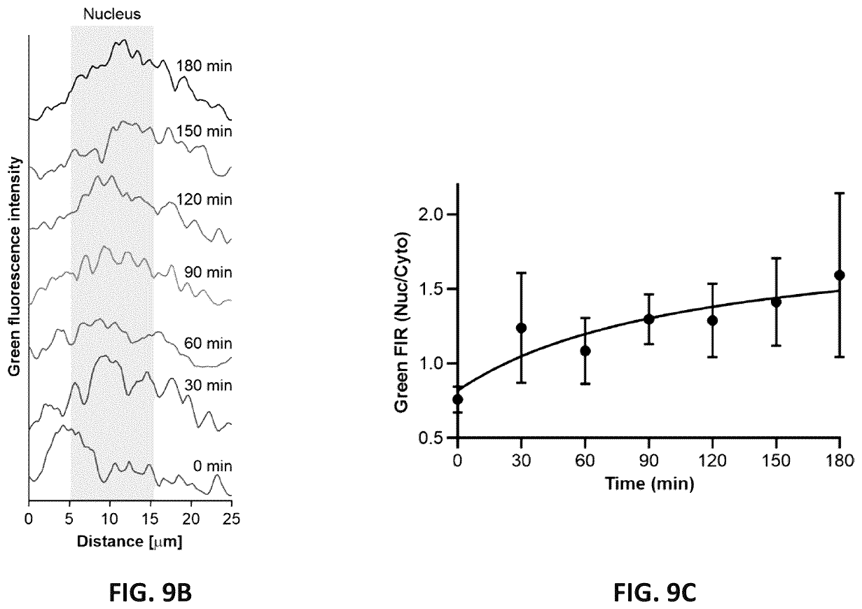

Finally, time-lapse imaging successfully demonstrated real-time monitoring of neuronal differentiation based on dynamic translocation of green fluorescence within live cellular environments (FIG. 9A). Notably, green fluorescence signal was gradually transported to the nucleus starting at approximately 24 h of differentiation. The focus in this study was on monitoring the sensing events from the first day of differentiation, and the green fluorescence signal was gradually translocated to the nucleus within 30 min after 1 day of differentiation (FIGS. 9B and 9C); these results are consistent with the time-dependent imaging results.

The above-described results represent a substantial advancement in comprehending and tracking the intricate progression of neural differentiation. The developed, non-invasive biosensing system provides immediate and accurate information about the process of neurogenesis via methodically monitoring hippocalcin levels and converting them into a captivating demonstration of fluorescence signal translocation. This dynamic readout provides opportunities not only for researching neural growth, but also for regenerative approaches for neurological injuries and assisting in the treatment of neurological diseases. The CBB demonstrated high sensitivity with a LOD of 42 nm, which indicates excellent performance for CBBs when considering that mature neurons typically express ≈3 μm hippocalcin. Furthermore, the CBB successfully detected neuronal differentiation and showed high consistency with TuJ1-positively stained cells using conventional IF staining. The developed CBB could also report neurogenesis from day 1 of differentiation, notably before TuJ1 expression, and the signal continuously increased throughout the differentiation process. All sensor cells that stained positive for TuJ1 showed green fluorescence translocation, whereas sensor cells that stained positive for GFAP and Olig2 did not show green fluorescence translocation. These results indicated that NSC-based CBBs could not only monitor early-stage neuronal differentiation, but also reliably distinguish between two different cell types (neurons and glial cells). Notably, the above-described sensing system enables non-invasive, stain-free, and real-time monitoring of neuronal differentiation without the need for cell disruption performed in conventional methods such as RT-PCR, IF, and western blotting.

Moreover, this novel NSC-based CBB can capture spatiotemporal dynamics at the single-cell level, providing a qualitative analysis of differentiation lineages. In particular, this CBB can serve as a monitoring tool to offer insights into 3D human stem cell-derived systems, including spheroid cultures and brain organoid models. 3D culture models provide unique opportunities for studying human disease while complementing animal models (E. R. Shamir et al., Nat. Rev. Mol. Cell Biol. 2014, 15, 647) and IF staining is a powerful tool for visualizing and analyzing protein expression within these systems used in disease modeling (L. Kuett et al. Nat. Cancer 2022, 3, 122 and W. Cho et al., Mol. Brain 2023, 16, 37). However, IgG antibodies, often used for IF staining, have a molecular weight of ˜150 kDa and can barely penetrate large 3D specimens (I. Smyrek et al., Biomed. Opt. Express 2017, 8, 484), making it impossible to accurately detect and report neurogenesis within 3D structures (J. B. Phillips, Axon Growth and Regener. 2014, 1162, 113). CBBs, in contrast to other approaches, are capable of monitoring neuronal differentiation simultaneously with no staining or destructive procedures. This means that the above-developed CBBs bring a significant improvement in one's capacity to examine and regulate neuronal development. Their non-invasive characteristics and ability to provide precise measurements can be used in the fields of cell treatment and research on neurodegenerative diseases. Thus, CBBs precisely detect specific neural cells for cell therapy, ensuring the utmost level of quality and effectiveness for these therapies.

### SEQUENCE LISTING

Sequence Listing
SEQ ID NO: 1 (GRIA2):
MQKIMHISVLLSPVLWGLIFGVSSNSIQIGGLFPRGADQEYSAFRVGMVQFSTSEFRLTPHIDNLEV
ANSFAVTNAFCSQFSRGVYAIFGFYDKKSVNTITSFCGTLHVSFITPSFPTDGTHPFVIQMRPDLKG
ALLSLIEYYQWDKFAYLYDSDRGLSTLQAVLDSAAEKKWQVTAINVGNINNDKKDEMYRSLFQD
LELKKERRVILDCERDKVNDIVDQVITIGKHVKGYHYIIANLGFTDGDLLKIQFGGANVSGFQIVD
YDDSLVSKFIERWSTLEEKEYPGAHTTTIKYTSALTYDAVQVMTEAFRNLRKQRIEISRRGNAGDC
LANPAVPWGQGVEIERALKQVQVEGLSGNIKFDQNGKRINYTINIMELKTNGPRKIGYWSEVDK
MVVTLTELPSGNDTSGLENKTVVVTTILESPYVMMKKNHEMLEGNERYEGYCVDLAAEIAKHC
GFKYKLTIVGDGKYGARDADTKIWNGMVGELVYGKADIAIAPLTITLVREEVIDFSKPFMSLGISI
MIKKPQKSKPGVFSFLDPLAYEIWMCIVFAYIGVSVVLFLVSRFSPYEWHTEEFEDGRETQSSESTN
EFGIFNSLWFSLGAFMQQGCDISPRSLSGRIVGGVWWFFTLIIISSYTANLAAFLTVERMVSPIESAE
DLSKQTEIAYGTLDSGSTKEFFRRSKIAVFDKMWTYMRSAEPSVFVRTTAEGVARVRKSKGKYAY
LLESTMNEYIEQRKPCDTMKVGGNLDSKGYGIATPKGSSLRNAVNLAVLKLNEQGLLDKLKNK
WWYDKGECGSGGGDSKEKTSALSLSNVAGVFYILVGGLGLAMLVALIEFCYKSRAEAKRMKVA
KNAQNINPSSSQNSQNFATYKEGYNVYGIESVKI
SEQ ID NO: 2 (AP2):
MTDSKYFTTNKKGEIFELKAELNNEKKEKRKEAVKKVIAAMTVGKDVSSLFPDVVNCMQTDNL
ELKKLVYLYLMNYAKSQPDMAIMAVNSFVKDCEDPNPLIRALAVRTMGCIRVDKITEYLCEPLRK
CLKDEDPYVRKTAAVCVAKLHDINAQMVEDQGFLDSLRDLIADSNPMVVANAVAALSEISESHPN
SNLLDLNPQNINKLLTALNECTEWGQIFILDCLSNYNPKDDREAQSICERVTPRLSHANSAVVLSA
VKVLMKFPELLPKDSDYYNMLLKKLAPPLVTLLSGEPEVQYVALRNINLIVQKRPEILKQEIKVFF
VKYNDPIYVKLEKLDIMIRLASQANIAQVLAELKEYATEVDVDFVRKAVRAIGRCAIKVEQSAER
CVSTLLDLIQTKVNYVVQEAIVVIRDIFRKYPNKYESIIATLCENLDSLDEPDARAAMIWIVGEYAE
RIDNADELLESFLEGFHDESTQVQLTLLTAIVKLFLKKPSETQELVQQVLSLATQDSDNPDLRDRG
YIYWRLLSTDPVTAKEVVLSEKPLISEETDLIEPTLLDELICHIGSLASVYHKPPNAFVEGSHGIHRK
HLPIHHGSTDAGDSPVGTTTATNLEQPQVIPSQGDLLGDLLNLDLGPPVNVPQVSSMQMGAVDLL
GGGLDSLLGSDLGGGIGGSPAVGQSFIPSSVPATFAPSPTPAVVSSGLNDLFELSTGIGMAPGGYVA
PKAVWLPAVKAKGLEISGTFTHRQGHIYMEMNFTNKALQHMTDFAIQFNKNSFGVIPSTPLAIHTP
LMPNQSIDVSLPLNTLGPVMKMEPLNNLQVAVKNNIDVFYFSCLIPLNVLFVEDGKMERQVFLAT
WKDIPNENELQFQIKECHLNADTVSSKLQNNNVYTIAKRNVEGQDMLYQSLKLTNGIWILAELRI
QPGNPNYTLSLKCRAPEVSQYIYQVYDSILKN
SEQ ID NO: 3 (NLS_{N} fragment of Xenopus laevis Nucleoplasmin)
KRPAATKKXX [X is any amino acid]
SEQ ID NO: 4 (NLS_{C} fragment of Xenopus laevis Nucleoplasmin)
XXGQAKKKKLD [X is any amino acid]
SEQ ID NO: 5 (NLS of Xenopus laevis Nucleoplasmin)
KRPAATKKAGQAKKKKLD
SEQ ID NO: 6 (NLS of Human RB)
KRSAEGSNPPKPLKKLR
SEQ ID NO: 7 (NLS_{N} fragment of Human RB)
KRSAEGS
SEQ ID NO: 8 (NLS_{C} fragment of Human RB)
NPPKPLKKLR
SEQ ID NO: 9 (NLS of Human p53)
KRALPNNTSSSPQPKKKP
SEQ ID NO: 10 (NLS_{N} fragment of Human p53)
KRALPNNTSSS
SEQ ID NO: 11 (NLS_{C} fragment of Human p53)
PQPKKKP
SEQ ID NO: 12 (Sce VMA N-intein)
CFAKGTNVLMADGSIECIENIEVGNKVMGKDGRPREVIKLPRGRETMYSVVQKSQHRAHKSDSS
REVPELLKFTCNATHELVVRTPRSVRRLSRTIKGVEYFEVITFEMGQKKAPDGRIVELVKEVSKSY
PISEGPERANELVESYRKASNKAYFEWTIEARDLSLLGSHVRKATYQTYAPILY
SEQ ID NO: 13 (Sce VMA C-intein)
MVLLNVLSKCAGSKKFRPAPAAAFARECRGFYFELQELKEDDYYGITLSDDSDHQFLLANQVVV
HNCFN
SEQ ID NO: 14 (Npu N-intein)
CLSYETEILTVEYGLLPIGKIVEKRIECTVYSVDNNGNIYTQPVAQWHDRGEQEVFEYCLEDGSLI
RATKDHKFMTVDGQMLPIDEIFERELDLMRVDNLPN
SEQ ID NO: 15 (Npu C-intein)
IKIATRKYLGKQNVYDIGVERDHNFALKNGFIASN
SEQ ID NO: 16 (Gp41-1 N-intein)
CLDLKTQVQTPQGMKEISNIQVGDLVLSNTGYNEVLNVFPKSKKKSYKITLEDGKEIICSEEHLFP
TQTGEMNISGGLKEGMCLYVKE
SEQ ID NO: 17 (Gp41-1 C-intein)
LKKILKIEELDERELIDIEVSGNHLFYANDILTHN
SEQ ID NO: 18 (CFN tripeptide sequence)
CFN
SEQ ID NO: 19 (NLS_{N}-N-intein-AP2):
MKRPAATKKHMCFAKGTNVLMADGSIECIENIEVGNKVMGKDGRPREVIKLPRGRETMYSVVQ
KSQHRAHKSDSSREVPELLKFTCNATHELVVRTPRSVRRLSRTIKGVEYFEVITFEMGQKKAPDG
RIVELVKEVSKSYPISEGPERANELVESYRKASNKAYFEWTIEARDLSLLGSHVRKATYQTYAPILY
SRTDSKYFTTNKKGEIFELKAELNNEKKEKRKEAVKKVIAAMTVGKDVSSLFPDVVNCMQTDNL
ELKKLVYLYLMNYAKSQPDMAIMAVNSFVKDCEDPNPLIRALAVRTMGCIRVDKITEYLCEPLRK
CLKDEDPYVRKTAAVCVAKLHDINAQMVEDQGFLDSLRDLIADSNPMVVANAVAALSEISESHPN
SNLLDLNPQNINKLLTALNECTEWGQIFILDCLSNYNPKDDREAQSICERVTPRLSHANSAVVLSA
VKVLMKFPELLPKDSDYYNMLLKKLAPPLVTLLSGEPEVQYVALRNINLIVQKRPEILKQEIKVFF
VKYNDPIYVKLEKLDIMIRLASQANIAQVLAELKEYATEVDVDFVRKAVRAIGRCAIKVEQSAER
CVSTLLDLIQTKVNYVVQEAIVVIRDIFRKYPNKYESIIATLCENLDSLDEPDARAAMIWIVGEYAE
RIDNADELLESFLEGFHDESTQVQLTLLTAIVKLFLKKPSETQELVQQVLSLATQDSDNPDLRDRG
YIYWRLLSTDPVTAKEVVLSEKPLISEETDLIEPTLLDELICHIGSLASVYHKPPNAFVEGSHGIHRK
HLPIHHGSTDAGDSPVGTTTATNLEQPQVIPSQGDLLGDLLNLDLGPPVNVPQVSSMQMGAVDLL
GGGLDSLLGSDLGGGIGGSPAVGQSFIPSSVPATFAPSPTPAVVSSGLNDLFELSTGIGMAPGGYVA
PKAVWLPAVKAKGLEISGTFTHRQGHIYMEMNFTNKALQHMTDFAIQFNKNSFGVIPSTPLAIHTP
LMPNQSIDVSLPLNTLGPVMKMEPLNNLQVAVKNNIDVFYFSCLIPLNVLFVEDGKMERQVFLAT
WKDIPNENELQFQIKECHLNADTVSSKLQNNNVYTIAKRNVEGQDMLYQSLKLINGIWILAELRI
QPGNPNYTLSLKCRAPEVSQYIYQVYDSILKNGSGATNFSLLKQAGDVEENPGPMVSKGEEDNM
AIIKEFMRFKVHMEGSVNGHEFEIEGEGEGRPYEGTQTAKLKVTKGGPLPFAWDILSPQFMYGSK
AYVKHPADIPDYLKLSFPEGFKWERVMNFEDGGVVTVTQDSSLQDGEFIYKVKLRGTNFPSDGP
VMQKKTMGWEASSERMYPEDGALKGEIKQRLKLKDGGHYDAEVKTTYKAKKPVQLPGAYNV
NIKLDITSHNEDYTIVEQYERAEGRHSTGGMDELYK
SEQ ID NO: 20 (GRIA2-C-intein-NLS_{C} fragment-EGFP)
MQKIMHISVLLSPVLWGLIFGVSSNSIQIGGLFPRGADQEYSAFRVGMVQFSTSEFRLTPHIDNLEV
ANSFAVTNAFCSQFSRGVYAIFGFYDKKSVNTITSFCGTLHVSFITPSFPTDGTHPFVIQMRPDLKG
ALLSLIEYYQWDKFAYLYDSDRGLSTLQAVLDSAAEKKWQVTAINVGNINNDKKDEMYRSLFQD
LELKKERRVILDCERDKVNDIVDQVITIGKHVKGYHYIIANLGFTDGDLLKIQFGGANVSGFQIVD
YDDSLVSKFIERWSTLEEKEYPGAHTTTIKYTSALTYDAVQVMTEAFRNLRKQRIEISRRGNAGDC
LANPAVPWGQGVEIERALKQVQVEGLSGNIKFDQNGKRINYTINIMELKTNGPRKIGYWSEVDK
MVVTLTELPSGNDTSGLENKTVVVTTILESPYVMMKKNHEMLEGNERYEGYCVDLAAEIAKHC
GFKYKLTIVGDGKYGARDADTKIWNGMVGELVYGKADIAIAPLTITLVREEVIDFSKPFMSLGISI
MIKKPQKSKPGVFSFLDPLAYEIWMCIVFAYIGVSVVLFLVSRFSPYEWHTEEFEDGRETQSSESTN
EFGIFNSLWFSLGAFMQQGCDISPRSLSGRIVGGVWWFFTLIIISSYTANLAAFLTVERMVSPIESAE
DLSKQTEIAYGTLDSGSTKEFFRRSKIAVFDKMWTYMRSAEPSVFVRTTAEGVARVRKSKGKYAY
LLESTMNEYIEQRKPCDTMKVGGNLDSKGYGIATPKGSSLRNAVNLAVLKLNEQGLLDKLKNK
WWYDKGECGSGGGDSKEKTSALSLSNVAGVFYILVGGLGLAMLVALIEFCYKSRAEAKRMKVA
KNAQNINPSSSQNSQNFATYKEGYNVYGIESVKITRMVLLNVLSKCAGSKKFRPAPAAAFARECR
GFYFELQELKEDDYYGITLSDDSDHQFLLANQVVVHNCFNVDGQAKKKKLDMVSKGEELFTGV
VPILVELDGDVNGHKFSVSGEGEGDATYGKLTLKFICTTGKLPVPWPTLVTTLTYGVQCFSRYPD
HMKQHDFFKSAMPEGYVQERTIFFKDDGNYKTRAEVKFEGDTLVNRIELKGIDFKEDGNILGHK
LEYNYNSHNVYIMADKQKNGIKVNFKIRHNIEDGSVQLADHYQQNTPIGDGPVLLPDNHYLSTQ
SALSKDPNEKRDHMVLLEFVTAAGITLGMDELYK
SEQ ID NO: 21 (n-NLS fragment-N-intein-AP2 P2A mCherry)
aatgtagtcttatgcaatactcttgtagtcttgcaacatggtaacgatgagttagcaacatgccttacaaggagagaaaaagcaccgtgcatgccgattg
gtggaagtaaggtggtacgatcgtgccttattaggaaggcaacagacgggtctgacatggattggacgaaccactgaattgccgcattgcagagatattg
tatttaagtgcctagctcgatacataaacgggtctctctggttagaccagatctgagcctgggagctctctggctaactagggaacccactgcttaagcc
tcaataaagcttgccttgagtgcttcaagtagtgtgtgcccgtctgttgtgtgactctggtaactagagatccctcagacccttttagtcagtgtggaaa
atctctagcagtggcgcccgaacagggacttgaaagcgaaagggaaaccagaggagctctctcgacgcaggactcggcttgctgaagcgcgcacggcaag
aggcgaggggcggcgactggtgagtacgccaaaaattttgactagcggaggctagaaggagagagatgggtgcgagagcgtcagtattaagcgggggaga
attagatcgcgatgggaaaaaattcggttaaggccagggggaaagaaaaaatataaattaaaacatatagtatgggcaagcagggagctagaacgattcg
cagttaatcctggcctgttagaaacatcagaaggctgtagacaaatactgggacagctacaaccatcccttcagacaggatcagaagaacttagatcatt
atataatacagtagcaaccctctattgtgtgcatcaaaggatagagataaaagacaccaaggaagctttagacaagatagaggaagagcaaaacaaaag
taagaccaccgcacagcaagcggccgctgatcttcagacctggaggaggagatatgagggacaattggagaagtgaattatataaatataaagtagtaa
aaattgaaccattaggagtagcacccaccaaggcaaagagaagagtggtgcagagagaaaaaagagcagtgggaataggagctttgttccttgggttct
tgggagcagcaggaagcactatgggcgcagcgtcaatgacgctgacggtacaggccagacaattattgtctggtatagtgcagcagcagaacaatttgc
tgagggctattgaggcgcaacagcatctgttgcaactcacagtctggggcatcaagcagctccaggcaagaatcctggctgtggaaagatacctaaagg
atcaacagctcctggggatttggggttgctctggaaaactcatttgcaccactgctgtgccttggaatgctagttggagtaataaatctctggaacagat
ttggaatcacacgacctggatggagtgggacagagaaattaacaattacacaagcttaatacactccttaattgaagaatcgcaaaaccagcaagaaaag
aatgaacaagaattattggaattagataaatgggcaagtttgtggaattggtttaacataacaaattggctgtggtatataaaattattcataatgatag
taggaggcttggtaggtttaagaatagtttttgctgtactttctatagtgaatagagttaggcagggatattcaccattatcgtttcagacccacctccc
aaccccgaggggacccgacaggcccgaaggaatagaagaagaaggtggagagagagacagagacagatccattcgattagtgaacggatctcgacggtat
cgctagcttttaaaagaaaaggggggattggggggtacagtgcaggggaaagaatagtagacataatagcaacagacatacaaactaaagaattacaaaa
acaaattacaaaaattcaaaattttactagtgattatcggatcaactttgtatagaaaagttggggttgcgccttttccaaggcagccctgggtttgcgc
agggacgcggctgctctgggcgtggttccgggaaacgcagcggcgccgaccctgggtctcgcacattcttcacgtccgttcgcagcgtcacccggatctt
cgccgctacccttgtgggccccccggcgacgcttcctgctccgcccctaagtcgggaaggttccttgcggttcgcggcgtgccggacgtgacaaacggaa
gccgcacgtctcactagtaccctcgcagacggacagcgccagggagcaatggcagcgcgccgaccgcgatgggctgtggccaatagcggctgctcagcag
ggcgcgccgagagcagcggccgggaaggggcggtgcgggaggcggggtgtggggcggtagtgtgggccctgttcctgcccgcgcggtgttccgcattct
gcaagcctccggagcgcacgtcggcagtcggctccctcgttgaccgaatcaccgacctctctccccaggcaagtttgtacaaaaaagcaggctgccac
catgaaacgtccggcggccaccaagaaacatatgtgctttgccaagggtaccaatgttttaatggcggatgggtctattgaatgtattgaaaacattgag
gttggtaataaggtcatgggtaaagatggcagacctcgtgaggtaattaaattgcccagaggaagagaaactatgtacagcgtcgtgcagaaaagtcagc
acagagcccacaaaagtgactcaagtcgtgaagtgccagaattactcaagtttacgtgtaatgcgacccatgagttggttgttagaacacctcgtagtgt
ccgccgtttgtctcgtaccattaagggtgtcgaatattttgaagttattacttttgagatgggccaaaagaaagcccccgacggtagaattgttgagctt
gtcaaggaagtttcaaagagctacccaatatctgaggggcctgagagagccaacgaattagtagaatcctatagaaaggcttcaaataaagcttattttg
agtggactattgaggccagagatctttctctgttgggttcccatgttcgtaaagctacctaccagacttacgctccaattctttattctagaactgactc
caagtatttcacaaccaataaaaaaggagaaatatttgaactaaaagctgaactcaacaatgaaaagaaagaaaagagaaaggaggctgtgaagaaagtg
attgctgctatgaccgtggggaaggatgttagttctctctttccagacgtagtgaactgtatgcagactgacaatctggaactaaagaagcttgtgtatc
tctacttgatgaactacgccaagagtcagccagacatggccatcatggctgtaaacagctttgtgaaggactgtgaagatcctaatcctttgattcgagc
cttggcagtcagaaccatggggtgcatccgggtagacaaaattacagaatatctctgtgagccgctccgcaagtgcttgaaggatgaggatccctatgtt
cggaaaacagcagcagtctgcgtggcaaaactccatgatatcaatgcccaaatggtggaagatcagggatttctggattctctacgggatctcatagcag
attcaaatccaatggtggtggctaatgccgtagcggcattatctgaaatcagtgagtctcacccaaacagcaacttacttgatctgaacccacagaacat
taataagctgctgacagccctgaatgaatgcactgaatggggccagattttcatcctggactgcctgtctaattacaaccctaaagatgatcgggaggct
cagagcatctgtgagcgggtaactccccggctatcccatgccaactcagcagtggtgctttcagcggtaaaagtcctaatgaagtttccagaattgttac
ctaaggattctgactactacaatatgctgctgaagaagttagcccctccacttgtcactttgctgtctggggagccagaagtgcagtatgtcgccctgag
gaacatcaacttaattgtccagaaaaggcctgaaatcttgaagcaggaaatcaaagtcttctttgtgaagtacaatgatcccatctatgttaaactagag
aagttggacatcatgattcgtttggcatctcaagccaacattgctcaggttctggcagaactgaaagaatatgctacagaggtggatgttgactttgttc
gaaaagctgtgcgggccattggacggtgtgccatcaaggtggagcaatctgcagagcgctgtgtaagcacattgcttgatctaatccagaccaaagtgaa
ttatgtggtccaagaagcaattgttgtcatcagggacatcttccgcaaataccccaacaagtatgaaagtatcatcgccactctgtgtgagaacttagac
tcgctggatgagccagatgctcgagcagctatgatttggattgtgggagaatatgctgaaagaattgacaatgcagatgagttactagaaagcttcctgg
agggttttcacgatgaaagcacccaggtgcagctcactctgcttactgccatagtgaagctgtttctcaagaaaccatcagaaacacaggagctagtcca
gcaggtcttgagtttggcaacacaggattctgataatcctgaccttcgagaccggggctatatttattggcgccttctctcaactgaccctgttacagct
aaagaagtagtcttgtctgagaagccactgatctctgaggagacggaccttattgagccaactctgctggatgagctaatctgccacattggttctttgg
cctctgtgtatcataagcctcccaatgcttttgtggaaggaagtcatggaattcatcgtaaacacttgccaattcatcatgggagcactgatgcaggtga
cagccctgttggcactaccactgcaacgaacctggaacagcctcaggttatcccctctcaaggtgatcttctaggggatcttttaaaccttgacctcggt
cccccagtcaatgtgccacaggtgtcctccatgcagatgggagcagtggatctcctaggaggaggactagatagtctgcttggcagtgaccttggcgggg
gcattggaggaagtccggcagtgggacaatccttcatcccatcatcggtccctgcaacctttgctccttcacctacacctgctgtggtcagcagtggact
gaatgacctgtttgaactctccacagggataggcatggcacctggtggatatgtggctcctaaggctgtctggctacctgcagtaaaggctaaaggcttg
gagatttccggaacatttactcaccgccaagggcacatctatatggaaatgaacttcaccaataaagctctgcagcacatgacagattttgcaatccagt
ttaacaaaaatagctttggtgtcatccccagcactcctctggccatccatacaccactgatgccaaaccagagcattgatgtctccctgcctctcaatac
cttgggcccagtcatgaagatggaacctctgaataacctccaggtggctgtgaaaaacaatatcgatgtcttctacttcagctgcctcatcccactcaat
gtgctttttgtagaagatggcaaaatggagcgccaggtcttccttgcaacatggaaggatattcccaatgaaaatgaacttcagtttcagattaaggaat
gtcatttaaatgctgacactgtttccagcaagttgcaaaacaacaatgtttatactattgccaagaggaatgtggaagggcaggacatgctgtaccaatc
cctgaagctcactaatggcatttggattttggccgaactacgtatccagccaggaaaccccaattacacgctgtcactgaagtgtagagctcctgaagtc
tctcaatacatctatcaggtctacgacagcattttgaaaaacggaagcggagccacgaacttctctctgttaaagcaagcaggagatgttgaagaaaacc
ccgggcctatggtgagcaagggcgaggaggataacatggccatcatcaaggagttcatgcgcttcaaggtgcacatggagggctccgtgaacggccacga
gttcgagatcgagggcgagggcgagggccgcccctacgagggcacccagaccgccaagctgaaggtgaccaagggtggccccctgcccttcgcctgggac
atcctgtcccctcagttcatgtacggctccaaggcctacgtgaagcaccccgccgacatccccgactacttgaagctgtccttccccgagggcttcaagt
gggagcgcgtgatgaacttcgaggacggcggcgtggtgaccgtgacccaggactcctccctgcaggacggcgagttcatctacaaggtgaagctgcgcgg
caccaacttcccctccgacggccccgtaatgcagaagaagaccatgggctgggaggcctcctccgagcggatgtaccccgaggacggcgccctgaagggc
gagatcaagcagaggctgaagctgaaggacggcggccactacgacgctgaggtcaagaccacctacaaggccaagaagcccgtgcagctgcccggcgcct
acaacgtcaacatcaagttggacatcacctcccacaacgaggactacaccatcgtggaacagtacgaacgcgccgagggccgccactccaccggcggcat
ggacgagctgtacaagtaaacccagctttcttgtacaaagtggtgataatcgaattccgataatcaacctctggattacaaaatttgtgaaagattgact
ggtattcttaactatgttgctccttttacgctatgtggatacgctgctttaatgcctttgtatcatgctattgcttcccgtatggctttcattttctcct
ccttgtataaatcctggttgctgtctctttatgaggagttgtggcccgttgtcaggcaacgtggcgtggtgtgcactgtgtttgctgacgcaacccccac
tggttggggcattgccaccacctgtcagctcctttccgggactttcgctttccccctccctattgccacggcggaactcatcgccgcctgccttgcccgc
tgctggacaggggctcggctgttgggcactgacaattccgtggtgttgtcggggaagctgacgtcctttccatggctgctcgcctgtgttgccacctgga
ttctgcgcgggacgtccttctgctacgtcccttcggccctcaatccagcggaccttccttcccgcggcctgctgccggctctgcggcctcttccgcgtct
tcgccttcgccctcagacgagtcggatctccctttgggccgcctccccgcatcgggaattcccgcggttcgaattctaccgggtaggggaggcgcttttc
ccaaggcagtctggagcatgcgctttagcagccccgctgggcacttggcgctacacaagtggcctctggcctcgcacacattccacatccaccggtaggc
gccaaccggctccgttctttggtggccccttcgcgccaccttctactcctcccctagtcaggaagttcccccccgccccgcagctcgcgtcgtgcaggac
gtgacaaatggaagtagcacgtctcactagtctcgtgcagatggacagcaccgctgagcaatggaagcgggtaggcctttggggcagcggccaatagcag
ctttgctccttcgctttctgggctcagaggctgggaaggggtgggtccgggggcgggctcaggggcgggctcaggggcggggcgggcgcccgaaggtcct
ccggaggcccggcattctgcacgcttcaaaagcgcacgtctgccgcgctgttctcctcttcctcatctccgggcctttcgacctcacgtgcgcatgattg
aacaagatggattgcacgcaggttctccggccgcttgggtggagaggctattcggctatgactgggcacaacagacaatcggctgctctgatgccgccgt
gttccggctgtcagcgcaggggcgcccggttctttttgtcaagaccgacctgtccggtgccctgaatgaactgcaagacgaggcagcgcggctatcgtgg
ctggccacgacgggcgttccttgcgcagctgtgctcgacgttgtcactgaagcgggaagggactggctgctattgggcgaagtgccggggcaggatctcc
tgtcatctcaccttgctcctgccgagaaagtatccatcatggctgatgcaatgcggcggctgcatacgcttgatccggctacctgcccattcgaccacca
agcgaaacatcgcatcgagcgagcacgtactcggatggaagccggtcttgtcgatcaggatgatctggacgaagagcatcaggggctcgcgccagccgaa
ctgttcgccaggctcaaggcgagcatgcccgacggcgaggatctcgtcgtgacccatggcgatgcctgcttgccgaatatcatggtggaaaatggccgct
tttctggattcatcgactgtggccggctgggtgtggcggaccgctatcaggacatagcgttggctacccgtgatattgctgaagagcttggcggcgaatg
ggctgaccgcttcctcgtgctttacggtatcgccgctcccgattcgcagcgcatcgccttctatcgccttcttgacgagttcttctgagcgggactctgg
gtacctttaagaccaatgacttacaaggcagctgtagatcttagccactttttaaaagaaaaggggggactggaagggctaattcactcccaacgaagac
aagatctgctttttgcttgtactgggtctctctggttagaccagatctgagcctgggagctctctggctaactagggaacccactgcttaagcctcaata
aagcttgccttgagtgcttcaagtagtgtgtgcccgtctgttgtgtgactctggtaactagagatccctcagacccttttagtcagtgtggaaaatctct
agcagtagtagttcatgtcatcttattattcagtatttataacttgcaaagaaatgaatatcagagagtgagaggaacttgtttattgcagcttataatg
gttacaaataaagcaatagcatcacaaatttcacaaataaagcatttttttcactgcattctagttgtggtttgtccaaactcatcaatgtatcttatca
tgtctggctctagctatcccgcccctaactccgcccatcccgcccctaactccgcccagttccgcccattctccgccccatggctgactaatttttttta
tttatgcagaggccgaggccgcctcggcctctgagctattccagaagtagtgaggaggcttttttggaggcctagggacgtacccaattcgccctatagt
gagtcgtattacgcgcgctcactggccgtcgttttacaacgtcgtgactgggaaaaccctggcgttacccaacttaatcgccttgcagcacatccccctt
tcgccagctggcgtaatagcgaagaggcccgcaccgatcgcccttcccaacagttgcgcagcctgaatggcgaatgggacgcgccctgtagcggcgcatt
aagcgcggcgggtgtggtggttacgcgcagcgtgaccgctacacttgccagcgccctagcgcccgctcctttcgctttcttcccttcctttctcgccacg
ttcgccggctttccccgtcaagctctaaatcgggggctccctttagggttccgatttagtgctttacggcacctcgaccccaaaaaacttgattagggtg
atggttcacgtagtgggccatcgccctgatagacggtttttcgccctttgacgttggagtccacgttctttaatagtggactcttgttccaaactggaac
aacactcaaccctatctcggtctattcttttgatttataagggattttgccgatttcggcctattggttaaaaaatgagctgatttaacaaaaatttaac
gcgaattttaacaaaatattaacgcttacaatttaggtggcacttttcggggaaatgtgcgcggaacccctatttgtttatttttctaaatacattcaaa
tatgtatccgctcatgagacaataaccctgataaatgcttcaataatattgaaaaaggaagagtatgagtattcaacatttccgtgtcgcccttattccc
ttttttgcggcattttgccttcctgtttttgctcacccagaaacgctggtgaaagtaaaagatgctgaagatcagttgggtgcacgagtgggttacatcg
aactggatctcaacagcggtaagatccttgagagttttcgccccgaagaacgttttccaatgatgagcacttttaaagttctgctatgtggcgcggtatt
atcccgtattgacgccgggcaagagcaactcggtcgccgcatacactattctcagaatgacttggttgagtactcaccagtcacagaaaagcatcttacg
gatggcatgacagtaagagaattatgcagtgctgccataaccatgagtgataacactgcggccaacttacttctgacaacgatcggaggaccgaaggagc
taaccgcttttttgcacaacatgggggatcatgtaactcgccttgatcgttgggaaccggagctgaatgaagccataccaaacgacgagcgtgacaccac
gatgcctgtagcaatggcaacaacgttgcgcaaactattaactggcgaactacttactctagcttcccggcaacaattaatagactggatggaggcggat
aaagttgcaggaccacttctgcgctcggcccttccggctggctggtttattgctgataaatctggagccggtgagcgtgggtctcgcggtatcattgcag
cactggggccagatggtaagccctcccgtatcgtagttatctacacgacggggagtcaggcaactatggatgaacgaaatagacagatcgctgagatagg
tgcctcactgattaagcattggtaactgtcagaccaagtttactcatatatactttagattgatttaaaacttcatttttaatttaaaaggatctaggtg
aagatcctttttgataatctcatgaccaaaatcccttaacgtgagttttcgttccactgagcgtcagaccccgtagaaaagatcaaaggatcttcttgag
atcctttttttctgcgcgtaatctgctgcttgcaaacaaaaaaaccaccgctaccagcggtggtttgtttgccggatcaagagctaccaactctttttcc
gaaggtaactggcttcagcagagcgcagataccaaatactgttcttctagtgtagccgtagttaggccaccacttcaagaactctgtagcaccgcctaca
tacctcgctctgctaatcctgttaccagtggctgctgccagtggcgataagtcgtgtcttaccgggttggactcaagacgatagttaccggataaggcgc
agcggtcgggctgaacggggggttcgtgcacacagcccagcttggagcgaacgacctacaccgaactgagatacctacagcgtgagctatgagaaagcgc
cacgcttcccgaagagagaaaggcggacaggtatccggtaagcggcagggtcggaacaggagagcgcacgagggagcttccagggggaaacgcctggtat
ctttatagtcctgtcgggtttcgccacctctgacttgagcgtcgatttttgtgatgctcgtcaggggggcggagcctatggaaaaacgccagcaacgcgg
cctttttacggttcctggccttttgctggccttttgctcacatgttctttcctgcgttatcccctgattctgtggataaccgtattaccgcctttgagtg
agctgataccgctcgccgcagccgaacgaccgagcgcagcgagtcagtgagcgaggaagcggaagagcgcccaatacgcaaaccgcctctccccgcgcgt
tggccgattcattaatgcagctggcacgacaggtttcccgactggaaagcgggcagtgagcgcaacgcaattaatgtgagttagctcactcattaggcac
cccaggctttacactttatgcttccggctcgtatgttgtgtggaattgtgagcggataacaatttcacacaggaaacagctatgaccatgattacgccaa
gcgcgcaattaaccctcactaaagggaacaaaagctggagctgcaagctt
SEQ ID NO: 22 (GRIA2-C-intein-c-NLS fragment-EGFP)
aatgtagtcttatgcaatactcttgtagtcttgcaacatggtaacgatgagttagcaacatgccttacaaggagagaaaaagcaccgtgcatgccgattg
gtggaagtaaggtggtacgatcgtgccttattaggaaggcaacagacgggtctgacatggattggacgaaccactgaattgccgcattgcagagatattg
tatttaagtgcctagctcgatacataaacgggtctctctggttagaccagatctgagcctgggagctctctggctaactagggaacccactgcttaagcc
tcaataaagcttgccttgagtgcttcaagtagtgtgtgcccgtctgttgtgtgactctggtaactagagatccctcagacccttttagtcagtgtggaaa
atctctagcagtggcgcccgaacagggacttgaaagcgaaagggaaaccagaggagctctctcgacgcaggactcggcttgctgaagcgcgcacggcaag
aggcgaggggcggcgactggtgagtacgccaaaaattttgactagcggaggctagaaggagagagatgggtgcgagagcgtcagtattaagcgggggaga
attagatcgcgatgggaaaaaattcggttaaggccagggggaaagaaaaaatataaattaaaacatatagtatgggcaagcagggagctagaacgattc
gcagttaatcctggcctgttagaaacatcagaaggctgtagacaaatactgggacagctacaaccatcccttcagacaggatcagaagaacttagatcat
tatataatacagtagcaaccctctattgtgtgcatcaaaggatagagataaaagacaccaaggaagctttagacaagatagaggaagagcaaaacaaaag
taagaccaccgcacagcaagcggccgctgatcttcagacctggaggaggagatatgagggacaattggagaagtgaattatataaatataaagtagtaa
aaattgaaccattaggagtagcacccaccaaggcaaagagaagagtggtgcagagagaaaaaagagcagtgggaataggagctttgttccttgggttct
tgggagcagcaggaagcactatgggcgcagcgtcaatgacgctgacggtacaggccagacaattattgtctggtatagtgcagcagcagaacaatttgc
tgagggctattgaggcgcaacagcatctgttgcaactcacagtctggggcatcaagcagctccaggcaagaatcctggctgtggaaagatacctaaagg
atcaacagctcctggggatttggggttgctctggaaaactcatttgcaccactgctgtgccttggaatgctagttggagtaataaatctctggaacagat
ttggaatcacacgacctggatggagtgggacagagaaattaacaattacacaagcttaatacactccttaattgaagaatcgcaaaaccagcaagaaaag
aatgaacaagaattattggaattagataaatgggcaagtttgtggaattggtttaacataacaaattggctgtggtatataaaattattcataatgatag
taggaggcttggtaggtttaagaatagtttttgctgtactttctatagtgaatagagttaggcagggatattcaccattatcgtttcagacccacctccc
aaccccgaggggacccgacaggcccgaaggaatagaagaagaaggtggagagagagacagagacagatccattcgattagtgaacggatctcgacggtat
cgctagcttttaaaagaaaaggggggattggggggtacagtgcaggggaaagaatagtagacataatagcaacagacatacaaactaaagaattacaaaa
acaaattacaaaaattcaaaattttactagtgattatcggatcaactttgtatagaaaagttgggctccggtgcccgtcagtgggcagagcgcacatcgc
ccacagtccccgagaagttggggggaggggtcggcaattgaaccggtgcctagagaaggtggcgcggggtaaactgggaaagtgatgtcgtgtactggct
ccgcctttttcccgagggtgggggagaaccgtatataagtgcagtagtcgccgtgaacgttctttttcgcaacgggtttgccgccagaacacaggtaagt
gccgtgtgtggttcccgcgggcctggcctctttacgggttatggcccttgcgtgccttgaattacttccacctggctgcagtacgtgattcttgatcccg
agcttcgggttggaagtgggtgggagagttcgaggccttgcgcttaaggagccccttcgcctcgtgcttgagttgaggcctggcctgggcgctggggccg
ccgcgtgcgaatctggtggcaccttcgcgcctgtctcgctgctttcgataagtctctagccatttaaaatttttgatgacctgctgcgacgctttttttc
tggcaagatagtcttgtaaatgcgggccaagatctgcacactggtatttcggtttttggggccgcgggcggcgacggggcccgtgcgtcccagcgcacat
gttcggcgaggcggggcctgcgagcgcggccaccgagaatcggacgggggtagtctcaagctggccggcctgctctggtgcctggtctcgcgccgccgtg
tatcgccccgccctgggcggcaaggctggcccggtcggcaccagttgcgtgagcggaaagatggccgcttcccggccctgctgcagggagctcaaaatgg
aggacgcggcgctcgggagagcggggggtgagtcacccacacaaaggaaaagggcctttccgtcctcagccgtcgcttcatgtgactccacggagtaccg
ggcgccgtccaggcacctcgattagttctcgagcttttggagtacgtcgtctttaggttggggggaggggttttatgcgatggagtttccccacactgag
tgggtggagactgaagttaggccagcttggcacttgatgtaattctccttggaatttgccctttttgagtttggatcttggttcattctcaagcctcaga
cagtggttcaaagtttttttcttccatttcaggtgtcgtgacaagtttgtacaaaaaagcaggctgccaccatgcaaaagattatgcatatttctgtcct
cctttctcctgttttatggggactgatttttggtgtctcttctaacagcatacagataggggggctatttcctaggggcgccgatcaagaatacagtgca
tttcgagtagggatggttcagttttccacttcggagttcagactgacaccccacatcgacaatttggaggtggcaaacagcttcgcagtcactaatgctt
tctgctcccagttttcgagaggagtctatgctatttttggattttatgacaagaagtctgtaaataccatcacatcattttgcggaacactccacgtctc
cttcatcactcccagcttcccaacagatggcacacatccatttgtcattcagatgagacccgacctcaaaggagctctccttagcttgattgaatactat
caatgggacaagtttgcatacctctatgacagtgacagaggcttatcaacactgcaagctgtgctggattctgctgctgaaaagaaatggcaagtgactg
ctatcaatgtgggaaacattaacaatgacaagaaagatgagatgtaccgatcactttttcaagatctggagttaaaaaaggaacggcgtgtaattctgga
ctgtgaaagggataaagtaaacgacattgtagaccaggttattaccattggaaaacatgttaaagggtaccactacatcattgcaaatctgggatttact
gatggagacctattaaaaatccagtttggaggtgcaaatgtctctggatttcagatagtggactatgatgattcgttggtatctaaatttatagaaagat
ggtcaacactggaagaaaaagaataccctggagctcacacaacaacaattaagtatacttctgctctgacctatgatgccgttcaagtgatgactgaagc
cttccgcaacctaaggaagcaaagaattgaaatctcccgaagggggaatgcaggagactgtctggcaaacccagcagtgccctggggacaaggtgtagaa
atagaaagggccctcaaacaggttcaggttgaaggtctctcaggaaatataaagtttgaccagaatggaaaaagaataaactatacaattaacatcatgg
agctcaaaactaatgggccccggaagattggctactggagtgaagtggacaaaatggttgttacccttactgagctcccttctggaaatgacacctctgg
gcttgagaataagactgttgttgtcaccacaattttggaatctccgtatgttatgatgaagaaaaatcatgaaatgcttgaaggcaatgagcgctatgag
ggctactgtgttgacctggctgcagaaatcgccaaacattgtgggttcaagtacaagttgacaattgttggtgatggcaagtatggggccagggatgcag
acacgaaaatttggaatgggatggttggagaacttgtatatgggaaagctgatattgcaattgctccattaactattacccttgtgagagaagaggtgat
tgacttctcaaagcccttcatgagcctcgggatatctatcatgatcaagaagcctcagaagtccaaaccaggagtgttttcctttcttgatcctttagcc
tatgagatctggatgtgcattgtttttgcctacattggggtcagtgtagttttattcctggtcagcagatttagcccctacgagtggcacactgaggagt
ttgaagatggaagagaaacacaaagtagtgaatcaactaatgaatttgggatttttaatagtctctggttttccttgggtgcctttatgcagcaaggatg
cgatatttcgccaagatccctctctgggcgcattgttggaggtgtgtggtggttctttaccctgatcataatctcctcctacacggctaacttagctgcc
ttcctgactgtagagaggatggtgtctcccatcgaaagtgctgaggatctttctaagcaaacagaaattgcttatggaacattagactctggctccacta
aagagtttttcaggagatctaaaattgcagtgtttgataaaatgtggacctacatgcggagtgcggagccctctgtgtttgtgaggactacggccgaagg
ggtggctagagtgcggaagtccaaagggaaatatgcctacttgttggagtccacgatgaacgagtacattgagcaaaggaagccttgcgacaccatgaaa
gttggtggaaacctggattccaaaggctatggcatcgcaacacctaaaggatcctcattaagaaatgcggttaacctcgcagtactaaaactgaatgaac
aaggcctgttggacaaattgaaaaacaaatggtggtacgacaaaggagagtgcggcagcgggggaggtgattccaaggaaaagaccagtgccctcagtct
gagcaacgttgctggagtattctacatccttgtcgggggccttggtttggcaatgctggtggctttgattgagttctgttacaagtcaagggccgaggcg
aaacgaatgaaggtggcaaagaatgcacagaatattaacccatcttcctcgcagaattcacagaattttgcaacttataaggaaggttacaacgtatatg
gcatcgaaagtgttaaaattacgcgtatggttttgcttaacgttctttcgaagtgtgccggctctaaaaaattcaggcctgctcccgccgctgcttttgc
acgtgagtgccgcggattttatttcgagttacaagaattgaaggaagacgattattatgggattactttatctgatgattctgatcatcagtttttgctt
gcaaaccaggttgtcgtccataactgtttcaacgtcgacggccaggcgaaaaagaagaaactggatatggtgagcaagggcgaggagctgttcaccgggg
tggtgcccatcctggtcgagctggacggcgacgtaaacggccacaagttcagcgtgtccggcgagggcgagggcgatgccacctacggcaagctgaccct
gaagttcatctgcaccaccggcaagctgcccgtgccctggcccaccctcgtgaccaccctgacctacggcgtgcagtgcttcagccgctaccccgaccac
atgaagcagcacgacttcttcaagtccgccatgcccgaaggctacgtccaggagcgcaccatcttcttcaaggacgacggcaactacaagacccgcgccg
aggtgaagttcgagggcgacaccctggtgaaccgcatcgagctgaagggcatcgacttcaaggaggacggcaacatcctggggcacaagctggagtacaa
ctacaacagccacaacgtctatatcatggccgacaagcagaagaacggcatcaaggtgaacttcaagatccgccacaacatcgaggacggcagcgtgcag
ctcgccgaccactaccagcagaacacccccatcggcgacggccccgtgctgctgcccgacaaccactacctgagcacccagtccgccctgagcaaagacc
ccaacgagaagcgcgatcacatggtcctgctggagttcgtgaccgccgccgggatcactctcggcatggacgagctgtacaagtaaacccagctttcttg
tacaaagtggtgataatcgaattccgataatcaacctctggattacaaaatttgtgaaagattgactggtattcttaactatgttgctccttttacgcta
tgtggatacgctgctttaatgcctttgtatcatgctattgcttcccgtatggctttcattttctcctccttgtataaatcctggttgctgtctctttatg
aggagttgtggcccgttgtcaggcaacgtggcgtggtgtgcactgtgtttgctgacgcaacccccactggttggggcattgccaccacctgtcagctcct
ttccgggactttcgctttccccctccctattgccacggcggaactcatcgccgcctgccttgcccgctgctggacaggggctcggctgttgggcactgac
aattccgtggtgttgtcggggaagctgacgtcctttccatggctgctcgcctgtgttgccacctggattctgcgcgggacgtccttctgctacgtccctt
cggccctcaatccagcggaccttccttcccgcggcctgctgccggctctgcggcctcttccgcgtcttcgccttcgccctcagacgagtcggatctccct
ttgggccgcctccccgcatcgggaattcccgcggttcgaattctaccgggtaggggaggcgcttttcccaaggcagtctggagcatgcgctttagcagcc
ccgctgggcacttggcgctacacaagtggcctctggcctcgcacacattccacatccaccggtaggcgccaaccggctccgttctttggtggccccttcg
cgccaccttctactcctcccctagtcaggaagttcccccccgccccgcagctcgcgtcgtgcaggacgtgacaaatggaagtagcacgtctcactagtct
cgtgcagatggacagcaccgctgagcaatggaagcgggtaggcctttggggcagcggccaatagcagctttgctccttcgctttctgggctcagaggctg
ggaaggggtgggtccgggggcgggctcaggggcgggctcaggggcggggcgggcgcccgaaggtcctccggaggcccggcattctgcacgcttcaaaagc
gcacgtctgccgcgctgttctcctcttcctcatctccgggcctttcgacctcacgtggccaccatgaccgagtacaagcccacggtgcgcctcgccaccc
gcgacgacgtccccagggccgtacgcaccctcgccgccgcgttcgccgactaccccgccacgcgccacaccgtcgatccggaccgccacatcgagcgggt
caccgagctgcaagaactcttcctcacgcgcgtcgggctcgacatcggcaaggtgtgggtcgcggacgacggcgccgcggtggcggtctggaccacgccg
gagagcgtcgaagcgggggcggtgttcgccgagatcggcccgcgcatggccgagttgagcggttcccggctggccgcgcagcaacagatggaaggcctcc
tggcgccgcaccggcccaaggagcccgcgtggttcctggccaccgtcggcgtctcgcccgaccaccagggcaagggtctgggcagcgccgtcgtgctccc
cggagtggaggcggccgagcgcgccggggtgcccgccttcctggagacctccgcgccccgcaacctccccttctacgagcggctcggcttcaccgtcacc
gccgacgtcgaggtgcccgaaggaccgcgcacctggtgcatgacccgcaagcccggtgcctgaggtacctttaagaccaatgacttacaaggcagctgta
gatcttagccactttttaaaagaaaaggggggactggaagggctaattcactcccaacgaagacaagatctgctttttgcttgtactgggtctctctggt
tagaccagatctgagcctgggagctctctggctaactagggaacccactgcttaagcctcaataaagcttgccttgagtgcttcaagtagtgtgtgcccg
tctgttgtgtgactctggtaactagagatccctcagacccttttagtcagtgtggaaaatctctagcagtagtagttcatgtcatcttattattcagtat
ttataacttgcaaagaaatgaatatcagagagtgagaggaacttgtttattgcagcttataatggttacaaataaagcaatagcatcacaaatttcacaa
ataaagcatttttttcactgcattctagttgtggtttgtccaaactcatcaatgtatcttatcatgtctggctctagctatcccgcccctaactccgccc
atcccgcccctaactccgcccagttccgcccattctccgccccatggctgactaattttttttatttatgcagaggccgaggccgcctcggcctctgagc
tattccagaagtagtgaggaggcttttttggaggcctagggacgtacccaattcgccctatagtgagtcgtattacgcgcgctcactggccgtcgtttta
caacgtcgtgactgggaaaaccctggcgttacccaacttaatcgccttgcagcacatccccctttcgccagctggcgtaatagcgaagaggcccgcaccg
atcgcccttcccaacagttgcgcagcctgaatggcgaatgggacgcgccctgtagcggcgcattaagcgcggcgggtgtggtggttacgcgcagcgtgac
cgctacacttgccagcgccctagcgcccgctcctttcgctttcttcccttcctttctcgccacgttcgccggctttccccgtcaagctctaaatcggggg
ctccctttagggttccgatttagtgctttacggcacctcgaccccaaaaaacttgattagggtgatggttcacgtagtgggccatcgccctgatagacgg
tttttcgccctttgacgttggagtccacgttctttaatagtggactcttgttccaaactggaacaacactcaaccctatctcggtctattcttttgattt
ataagggattttgccgatttcggcctattggttaaaaaatgagctgatttaacaaaaatttaacgcgaattttaacaaaatattaacgcttacaatttag
gtggcacttttcggggaaatgtgcgcggaacccctatttgtttatttttctaaatacattcaaatatgtatccgctcatgagacaataaccctgataaat
gcttcaataatattgaaaaaggaagagtatgagtattcaacatttccgtgtcgcccttattcccttttttgcggcattttgccttcctgtttttgctcac
ccagaaacgctggtgaaagtaaaagatgctgaagatcagttgggtgcacgagtgggttacatcgaactggatctcaacagcggtaagatccttgagagtt
ttcgccccgaagaacgttttccaatgatgagcacttttaaagttctgctatgtggcgcggtattatcccgtattgacgccgggcaagagcaactcggtcg
ccgcatacactattctcagaatgacttggttgagtactcaccagtcacagaaaagcatcttacggatggcatgacagtaagagaattatgcagtgctgcc
ataaccatgagtgataacactgcggccaacttacttctgacaacgatcggaggaccgaaggagctaaccgcttttttgcacaacatgggggatcatgtaa
ctcgccttgatcgttgggaaccggagctgaatgaagccataccaaacgacgagcgtgacaccacgatgcctgtagcaatggcaacaacgttgcgcaaact
attaactggcgaactacttactctagcttcccggcaacaattaatagactggatggaggcggataaagttgcaggaccacttctgcgctcggcccttccg
gctggctggtttattgctgataaatctggagccggtgagcgtgggtctcgcggtatcattgcagcactggggccagatggtaagccctcccgtatcgtag
ttatctacacgacggggagtcaggcaactatggatgaacgaaatagacagatcgctgagataggtgcctcactgattaagcattggtaactgtcagacca
agtttactcatatatactttagattgatttaaaacttcatttttaatttaaaaggatctaggtgaagatcctttttgataatctcatgaccaaaatccct
taacgtgagttttcgttccactgagcgtcagaccccgtagaaaagatcaaaggatcttcttgagatcctttttttctgcgcgtaatctgctgcttgcaaa
caaaaaaaccaccgctaccagcggtggtttgtttgccggatcaagagctaccaactctttttccgaaggtaactggcttcagcagagcgcagataccaaa
tactgttcttctagtgtagccgtagttaggccaccacttcaagaactctgtagcaccgcctacatacctcgctctgctaatcctgttaccagtggctgct
gccagtggcgataagtcgtgtcttaccgggttggactcaagacgatagttaccggataaggcgcagcggtcgggctgaacggggggttcgtgcacacagc
ccagcttggagcgaacgacctacaccgaactgagatacctacagcgtgagctatgagaaagcgccacgcttcccgaagagagaaaggcggacaggtatcc
ggtaagcggcagggtcggaacaggagagcgcacgagggagcttccagggggaaacgcctggtatctttatagtcctgtcgggtttcgccacctctgactt
gagcgtcgatttttgtgatgctcgtcaggggggcggagcctatggaaaaacgccagcaacgcggcctttttacggttcctggccttttgctggccttttg
ctcacatgttctttcctgcgttatcccctgattctgtggataaccgtattaccgcctttgagtgagctgataccgctcgccgcagccgaacgaccgagcg
cagcgagtcagtgagcgaggaagcggaagagcgcccaatacgcaaaccgcctctccccgcgcgttggccgattcattaatgcagctggcacgacaggttt
cccgactggaaagcgggcagtgagcgcaacgcaattaatgtgagttagctcactcattaggcaccccaggctttacactttatgcttccggctcgtatgt
tgtgtggaattgtgagcggataacaatttcacacaggaaacagctatgaccatgattacgccaagcgcgcaattaaccctcactaaagggaacaaaagct
ggagctgcaagctt
SEQ ID NO: 23 (P2A)
GSGATNFSLLKQAGDVEENPGP
SEQ ID NO: 24 (PPT)
AAAAGAAAAGGGGGG
SEQ ID NO: 25 (AgeI SpeI IC-NLS₁₀₋₁₈-F)
GAGTGTTCACCGGTACTAGTATGGTTTT
SEQ ID NO: 26 (IC-NLS₁₀₋₁₈ ApaI-R)
TCACCATGGGCCCATCCAGTTTC
SEQ ID NO: 27 (ApaI EGFP-F)
GGGCCCATGGTGAGCAAGGGC
SEQ ID NO: 28 (EGFP XbaI-R)
TCTAGATTACTTGTACAGCTCGTCC
SEQ ID NO: 29 (AgeI Kozak GRIA2-F)
GTGGATGCTACCGGTACCATGGAAATG
SEQ ID NO: 30 (GRIA2 SpeI-R)
CAAGGTCATACTAGTAATTTTAACACTT
SEQ ID NO: 31 (SalI AP2-F)
CACATTAAAGATGTCGACTCATGACTGACTCCAAG
SEQ ID NO: 32 (AP2 EagI-R)
GTACTGGACCACGGCCGTTAGTTTTTCAAAATGC
SEQ ID NO: 33 (MluI NLS₁₋₉-IN-AP2-F)
GCGGCACGCGTATGAAACGTCCGGC
SEQ ID NO: 34 (NLS₁₋₉-IN-AP2 EagI-R)
GAGTGCGGCCGTTAGTTTTTCAAAATGC
SEQ ID NO: 35 (SalI mNLS BamHI-F)
TCGACATGAAACGTCCGGCGGCCACCAAGAAAGCTAGCTGTTTCAACGCTAGCGGCCAGGCG
AAAAAGAAGAAACTG
SEQ ID NO: 36 (SalI mNLS BamHI-R)
CAGTTTCTTCTTTTTCGCCTGGCCGCTAGCGTTGAAACAGCTAGCTTTCTTGGTGGCCGCCGG
ACGTTTCATGTCGA
SEQ ID NO: 37 (HindIII hippocalcin-F)
GGCTCGAAGCTTATGGGCAAGCAG
SEQ ID NO: 38 (hippocalcin EcoRV-R)
GAACCTGGGATATCTCAGAACTGGG
SEQ ID NO: 39 (GAPDH F primer)
CCGCATCTTCTTTTGCGTCG
SEQ ID NO: 40 (GAPDH R primer)
GCCCAATACGACCAAATCCGT
SEQ ID NO: 41 (SOX2 F primer)
CGAGTGGAAACTTTTGTCGG
SEQ ID NO: 42 (SOX2 R primer)
TCTTCATGAGCGTCTTGGTT
SEQ ID NO: 43 (TUBB3 F primer)
GGACCCTGTGAGTAGCCAGTA
SEQ ID NO: 44 (TUBB3 R primer)
CAGCTCCGACAGATCCAGT
SEQ ID NO: 45 (NEUROD1 F primer)
GTCTCCTTCGTTCAGACGCTT
SEQ ID NO: 46 (NEUROD1 R primer)
AAAGTCCGAGGATTGAGTTGC
SEQ ID NO: 47 (HPCA F primer)
AGATGGTTTCGTCCGTGATG
SEQ ID NO: 48 (HPCA R primer)
CTTGCCGTCGTTGTTTGTG
SEQ ID NO: 49 (M2 to CTD of GRIA2)
YEIWMCIVFAYIGVSVVLFLVSRFSPYEWHTEEFEDGRETQSSESTNEFGIFNSLWFSLGAFMQ
QGCDISPRSLSGRIVGGVWWFFTLIIISSYTANLAAFLTVERMVSPIESAEDLSKQTEIAYGTLD
SGSTKEFFRRSKIAVFDKMWTYMRSAEPSVFVRTTAEGVARVRKSKGKYAYLLESTMNEYIE
QRKPCDTMKVGGNLDSKGYGIATPKGSSLRNAVNLAVLKLNEQGLLDKLKNKWWYDKGEC
GSGGGDSKEKTSALSLSNVAGVFYILVGGLGLAMLVALIEFCYKSRAEAKRMKVAKNAQNIN
PSSSQNSQNFATYKEGYNVYGIESVKI
SEQ ID NO: 50 (M3 to CTD of GRIA2)
FGIFNSLWFSLGAFMQQGCDISPRSLSGRIVGGVWWFFTLIIISSYTANLAAFLTVERMVSPIES
AEDLSKQTEIAYGTLDSGSTKEFFRRSKIAVFDKMWTYMRSAEPSVFVRTTAEGVARVRKSK
GKYAYLLESTMNEYIEQRKPCDTMKVGGNLDSKGYGIATPKGSSLRNAVNLAVLKLNEQGLL
DKLKNKWWYDKGECGSGGGDSKEKTSALSLSNVAGVFYILVGGLGLAMLVALIEFCYKSRA
EAKRMKVAKNAQNINPSSSQNSQNFATYKEGYNVYGIESVKI
SEQ ID NO: 51 (M4 to CTD of GRIA2)
VAGVFYILVGGLGLAMLVALIEFCYKSRAEAKRMKVAKNAQNINPSSSQNSQNFATYKEGYN
VYGIESVKI
SEQ ID NO: 52 (EGFP)
MVSKGEELFTGVVPILVELDGDVNGHKFSVSGEGEGDATYGKLTLKFICTTGKLPVPWPTLVT
TLTYGVQCFSRYPDHMKQHDFFKSAMPEGYVQERTIFFKDDGNYKTRAEVKFEGDTLVNRIE
LKGIDFKEDGNILGHKLEYNYNSHNVYIMADKQKNGIKVNFKIRHNIEDGSVQLADHYQQN
TPIGDGPVLLPDNHYLSTQSALSKDPNEKRDHMVLLEFVTAAGITLGMDELYK

What is claimed is:

1. A biosensor protein comprising a C-fusion protein and an N-fusion protein, wherein
i. the C-fusion protein comprises a nuclear localization signal (NLS), a C-intein, CFN tripeptide sequence (SEQ ID NO: 18), a glutamate ionotropic receptor AMPA type subunit 2 (GRIA2), and a reporter molecule; and
ii. the N-fusion protein comprises an NLS, an N-intein, and an Adaptor Protein Complex 2 (AP2).

2. The biosensor protein of claim 1, wherein the GRIA2 comprises the sequence of SEQ ID NO: 1, or the AP2 comprises the sequence of SEQ ID NO: 2, or the C-intein comprises the sequence of any one of SEQ ID NOs: 13, 15, or 17, or N-intein comprises the sequence of any one of SEQ ID NOs: 12, 14, or 16.

3. The biosensor protein of claim 1, wherein the NLS sequence of the N-fusion protein comprises the sequence of any one of SEQ ID NOs: 3, 7, and 10, or the NLS sequence of the C-fusion protein comprises the sequence of any one of SEQ ID NOs: 4, 8, and 11.

4. The biosensor protein of claim 1, wherein the reporter molecule is a fluorescent protein.

5. The biosensor protein of claim 4, wherein the fluorescent protein comprises yellow fluorescent protein (YFP), green fluorescent protein (GFP), mCherry, red fluorescent protein (RFP), or blue fluorescent protein (BFP).

6. The biosensor protein of claim 1, wherein the reporter molecule protein is inserted at the C-terminus of the NLS of the C-fusion protein.

7. The biosensor protein of claim 1, wherein the N-fusion protein further comprises a fluorescent protein inserted at the C-terminus of AP2.

8. The biosensor protein of claim 1, wherein AP2 of the N-fusion protein binds to hippocalcin (HPCA).

9. The biosensor protein of claim 8, wherein the C-fusion and the N-fusion protein heterodimerize in response to hippocalcin binding AP2 of the C-fusion protein.

10. An isolated nucleic acid molecule encoding the biosensor protein of claim 1.

11. An expression vector or a host cell comprising the nucleic acid molecule of claim 14.

12. The host cell of claim 15, wherein the cell is a stem cell.

13. The host cell of claim 16, wherein the cell is a human induced pluripotent stem cell (hiPSC)-derived neural progenitor cell (NPC).

14. A cell-based biosensor (CBB) that expresses:
i. a C-fusion protein, wherein the C-fusion protein comprises a nuclear localization signal (NLS), a C-intein, CFN tripeptide sequence (SEQ ID NO: 18), a glutamate ionotropic receptor AMPA type subunit 2 (GRIA2), and a reporter molecule; and
ii. an N-fusion protein, wherein the N-fusion protein comprises an NLS, an N-intein, and an Adaptor Protein Complex 2 (AP2).

15. The CBB of claim 14, wherein the GRIA2 comprises the sequence of SEQ ID NO: 1, or the AP2 comprises the sequence of SEQ ID NO: 2, or the C-intein comprises the sequence of any one of SEQ ID NOs: 13, 15, or 17, or N-intein comprises the sequence of any one of SEQ ID NOs: 12, 14, or 16.

16. The CBB of claim 14, wherein the NLS sequence of the N-fusion protein comprises the sequence of any one of SEQ ID NOs: 3, 7, and 10, or the NLS sequence of the C-fusion protein comprises the sequence of any one of SEQ ID NOs: 4, 8, and 11.

17. The CBB of claim 14, wherein the reporter molecule is a fluorescent protein.

18. The CBB of claim 14, wherein the fluorescent protein comprises yellow fluorescent protein (YFP), green fluorescent protein (GFP), mCherry, red fluorescent protein (RFP), or blue fluorescent protein (BFP).

19. The CBB of claim 14, wherein the CBB detects hippocalcin.

20. A method for monitoring neuronal differentiation, the method comprising imaging a cell-based biosensor (CBB), wherein the CBB expresses:
i. a C-fusion protein, wherein the C-fusion protein comprises a nuclear localization signal (NLS), a C-intein, CFN tripeptide sequence (SEQ ID NO: 18), a glutamate ionotropic receptor AMPA type subunit 2 (GRIA2), and a reporter molecule; and
ii. an N-fusion protein, wherein the N-fusion protein comprises an NLS, an N-intein, and an Adaptor Protein Complex 2 (AP2); and
wherein the CBB detects hippocalcin expression.
